(19) United States
(12) Patent Application Publication
(10) Pub. No.: US 2008/0153872 A1
(43) Pub. Date: Jun. 26, 2008

(54) Bis-(Coumarin) Compounds With Anti-Inflammatory Activity

(76) Inventors: Mladen Mercep, Zagreb (HR); Ivica Malnar, Gerovo (HR); Boska Hrvacic, Zagreb (HR); Stribor Markovic, Zagreb (HR); Anita Filipovic Sucic, Zagreb (HR); Berislav Bosnjak, Zagreb (HR); Andreja Cempuh Klonkay, Zagreb (HR); Renata Rupcic, Zagreb (HR); Antun Hutinec, Zagreb (HR); Ivaylo Jivkov Elenkov, Zagreb (HR); Milan Mesic, Zagreb (HR)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/813,885

(22) PCT Filed: Jan. 13, 2006
(86) PCT No.: PCT/IB06/01259

§ 371 Date: Jul. 13, 2007

Related U.S. Application Data
(60) Provisional application No. 60/644,359, filed on Jan. 14, 2005.
(60) Provisional application No. 60/647,793, filed on Jan. 27, 2005.

(51) Int. Cl. A61K 31/4709; C07D 407/06; A61K 31/353; A61K 31/357; C07D 409/14; A61K 31/4406; C07D 493/14; A61P 37/00; A61P 29/00; C07D 493/04; A61K 31/352; C07D 401/14; A61K 31/381; C07D 405/14; A61K 31/4155

(52) U.S. Cl. 514/314; 549/400; 514/457; 548/311.4; 514/402; 546/174; 549/378; 514/452; 514/444; 549/60; 546/283.1; 514/337; 514/453; 549/382; 549/387; 514/455

(57) ABSTRACT

Certain bis-(coumarin) compounds as well as the products of their intramolecular cyclization including pharmaceutically acceptable salts, hydrates, solvates, clathrates, prodrugs, tautomers and stereoisomers thereof are disclosed. Certain processes and intermediates for the preparation of certain bis-(coumarin) compounds, as well as for the use of these compounds as therapeutically active agents in the prophylaxis and treatment of asthma and other inflammatory diseases and conditions in mammals, especially humans are also disclosed.

### FIELD OF THE INVENTION

The present invention relates to certain bis-(coumarin) compounds as well as the products of their intramolecular cyclization including pharmaceutically acceptable salts, solvates (including hydrates), clathrates, prodrugs, tautomers and stereoisomers thereof. The invention further relates to processes and intermediates for the preparation of certain bis-(coumarin) compounds, as well as to the use of these compounds as therapeutically active agents in the prophylaxis and treatment of asthma and other inflammatory diseases and conditions in mammals, especially humans.

### TECHNICAL PROBLEM

The present invention is responsive to the technical problem of providing novel anti-inflammatory agents that have pronounced activity against inflammatory conditions or disorders, that are more effective against specific types of inflammation disorders than steroids or NSAIDs and/or that have an improved side effect profile compared to heretofore known PDE4 inhibitors, or leukotriene inhibitors.

### BACKGROUND OF THE INVENTION

Asthma is a chronic inflammatory disease of the respiratory airways in mammals. Clinically, in hypersensitive persons the inflammation causes periodic coughing attacks, troubled breathing, wheezing, tightness in the chest and chest pain. The inflammation makes respiratory airways more susceptible to irritations by allergens, chemical irritants, tobacco smoke, cold air and strain. Exposed to these irritants, respiratory airways become edematous, contracted, filled with mucus and hypersensitive.

The pathogenesis of asthma is complex and includes the interaction of inflammatory cells, mediators as well as of the tissue and cells of respiratory airways. In the asthmatic process an early phase and a late phase of a response are distinguished. Allergic diseases as well as allergen-induced asthma are characterised by the synthesis of a specific type of IgE antibodies. Immediately after the inhalation of allergens, complexes of allergens and allergen specific IgE's are bound to high affinity IgE receptor (Fcs receptor type I) present on basophils, mastocytes and eosinophils. By the binding to the receptor the activation of signal transfer cascade occurs, which results in:

1. de novo synthesis of proinflammatory genes (e.g. interleukin-4 and interleukin-5),

2. exocytosis of the content of cytoplasmatic granules-degranulation.

The granules contain inflammatory mediators such as histamine, serotonin, leukotrienes C4, D4 and E4, and proteins such as major basic protein and mieloperoxidase. These inflammatory mediators co-operate in the processes of vasodilation, bronchoconstriction, triggering and control of the inflammatory process and activation of the cells and damage to the inflamed tissue. These processes form the early asthmatic response. The inhibition of degranulation may prevent the symptoms and stop the inflammation progress, which has been proven by the clinical use of degranulation inhibitors (sodium cromoglycate, nedocromil sodium and ketotifen).

The late asthmatic response includes a permanent obstruction of air passages, a hyperreactivity of the bronchi and a development of inflammation changes including the accumulation of neutrophils, eosinophils, lymphocytes and monocytes/macrophages in the respiratory system. The accumulation of inflammatory cells results from hamonized interaction of lymphokines (TNF-α, IL-4, IL-5), adhesion molecules on the surface of leukocytes (integrins) and endothelial cells (selectins), and chemokins (eotaxin, RANTES). The role and significance of T-lymphocytes in asthma were confirmed by the existence of an increased number of activated CD4+ T-cells in bronchoalveolar lavage and bronchial biopsies of patients suffering from asthma. Two subpopulations of CD4+ cells differ with regard to the profile of cytokines they secrete. Th 1 cells secrete IL-2, IL-3, GM-CSF, INF-γ. Activation of Th 1 cells is important in the defense of the host against intracellular organisms, viruses and neoplasms. Investigations have demonstrated that, in asthma, Th 2 cell response prevails with an increased expression of IL-5 that is important in the formation of eosinophilic infiltration typical of allergic inflammation.

Morphologic changes occurring in asthma include an infiltration of the bronchi by inflammation cells (mastocytes, T-lymphocytes and eosinophils are the key executive cells), a clogging of respiratory airways by a secrete, interstition oedema and increased microcirculation permeability. On the basis of pathohistological findings it has been established that eosinophilic infiltration is specific and differentiates asthma from other types of inflammation.

In the control of asthma two types of medicaments exists, symptomatic ones and basic ones. The symptomatic medicaments include short-acting bronchodilators such as β2-agonists, anticholinergics, theophylline, which rapidly relax the contracted respiratory airways and alleviate the acute symptoms. The basic medicaments include antiinflammatory drugs and long-acting bronchodilators. Antiinflammatory drugs alleviate and prevent the inflammation reaction and they include inhalable corticosteroids, systemic corticosteroids and inhalable cromones, such as cromolyn and nedocromil.

Steroid antiinflammatory compounds are still considered to be the most effective medicaments in the treatment of inflammatory diseases and conditions such as asthma. The good potency and efficacy of this type of medicament are, however, accompanied by numerous undesired side effects, such as disturbances of carbohydrate metabolism, of calcium resorption, of the secretion of endogenic corticosteroids and of physiological functions of the hypophysis, of the suprarenal gland core and of the thymus. In the literature (WO 94/13690, WO 94/14834, WO 92/13872 and WO 92/13873) so-called “soft” steroids or hydrolysable corticosteroids with local action are described. Their systemic, undesired effect is reduced due to the instability of “soft” steroids in serum, where the active steroid is rapidly hydrolyzed to an inactive form. However, a steroid without negative side effects in long-term use has yet to be found.

New medications concentrate on reducing inflammatory response as a method for alleviating or eliminating symptoms (Barnes, P. J. Nature Reviews Drug Discovery, 2004, 3, 831-844; Bals, R. Curr. Med. Chem.—Anti-Inflammatory & Anti-Allergy Agents, 2004, 3, 39-52; Coruzzi, G. Current Drug Targets—Inflammation & Allergy, 2004, 3, 43-61; Prescott, S. L. Med. Chem. Reviews—Online, 2004, 1, 163-177). The leukotriene inhibitors are the newest addition to the asthma therapy. Leukotrienes are produced by 5-lipoxygenase enzyme pathway, and inhibitors are divided into several groups according to their mode of action. Cysteinyl leukotriene antagonists have proven efficacy in short and long-term studies: improvements in baseline lung function in asthmatics and improvement of a variety of symptoms as well as decreased use of β2-agonists and a reduction in asthma exacerbations have been reported (Barreiro, E. J. Curr. Med. Chem.—Anti-Inflammatory & Anti-Allergy Agents, 2004, 3, 9-18). Therapeutically, the 5-lipoxygenase inhibitor, zileuton, is as effective as the cysteinyl leulcotrienle antagonists, and its therapeutic effects are indistinguishable from those of the cysteinyl leulcotriene antagonists. Several LTB4 antagonists are currently under investigation aimed at different indications (Balazy, M. Current Drug Targets—Inflammation & Allergy, 2004, 3, 19-33). Leulcotriene inhibitors represent an advance over current anti-asthma products in terms of faster onset of action, oral formulation and favorable side effect profile.

The phosphodiesterases (PDEs) are responsible for the hydrolysis of intracellular cyclic adenosine and guanosine monophosphate (cAMP and cGMP, respectively). Type 4 phosphodiesterase (PDE4) is a cAMP-specific enzyme localized in airway smooth muscle cells as well as in immune and inflammatory cells. The PDE4 activity is associated with a wide variety of diseases some of which have been related to an inflammatory state, e.g. asthma, chronic obstructive pulmonary disease (COPD), rheumatoid arthritis (RA), while others have recently been connected to autoimmune pathology. Therefore, an intense effort towards the development of PDE4 inhibitors has been generated for the last decade (Giembycz, M. A. Drugs, 2000, 59, 193-212; Lipworth, B. Lancet, 2005, 365, 167-175). Unfortunately, the effects of prototype PDE4 inhibitors have been compromised by side effects such as nausea and emesis and the clinical use of those compounds is still limited.

Some compounds of the coumarin class are known to inhibit the immunological release of chemical mediators such as SRS-A (the slow reacting substance of anaphylaxis) and histamine from mast cells (U.S. Pat. No. 4,731,375). Other compounds of the coumarin class are known to not only protect against the release of SRS-A and other mediators of the allergic response but to also inhibit the action of SRS-A (U.S. Pat. No. 4,200,577). Still other compounds of the coumarin class are known to not only inhibit the release of mediator substances but to also antagonize the effects of histamine released after the antibody-antigen combinations (U.S. Pat. No. 4,263,299). Similarly, others inhibit the effects of certain types of antigen-antibody reactions (U.S. Pat. No. 4,059,704), have NMDA (N-methyl-D-aspartate) antagonistic action (U.S. Pat. No. 5,428,038), inhibit leukotriene biosynthesis (U.S. Pat. No. 5,576,338), modulate the histamine-3 receptors (US 2002/0183309), and inhibit phosphodiesterase VII (US 2004/0138279). Therefore, the coumarin compounds described are useful in the prophylaxis and treatment of various diseases associated with allergic or immunological reactions such as allergic asthma, allergic dermatitis, allergic rhinitis or enteritis, allergic conjunctivitis or allergic eczema.

There remains a need to develop compounds of the coumarin class for the treatment of inflammatory disorders with greater efficacy and potency while avoiding the side effects of current therapies.

### SUMMARY OF THE INVENTION

The present invention relates to bis-(coumarin) compounds with valuable properties, in particular pharmacological properties for treating inflammatory diseases and conditions such as asthma, and which can also be used to produce pharmaceuticals.

The present invention relates to bis-(coumarin) compounds of Formula I,

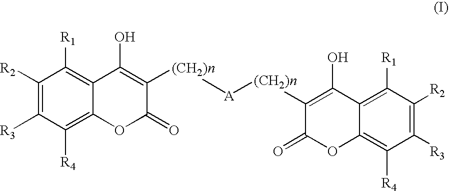

as well as the products of their intramolecular cyclization having Formulas II and III,

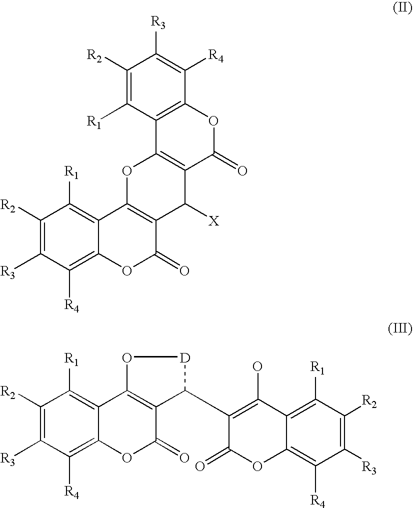

and pharmaceutically acceptable salts, solvates (including hydrates), clathrates, prodrugs and tautomers and stereoisomers thereof wherein:
R₁, R₂, R₃ and R₄, are each independently hydrogen, fluoro, chloro, bromo, C₁-C₄-alkyl, C₂-C₄-alkenyl, C₂-C₄-alkynyl, halo-C₁-C₄-alkyl, hydroxy, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro;
A is carbonyl, CH—X or C═N—R₅;
Each occurrence of n is, independently, an integer which is 0 or 1;
R₅ is an hydroxy, alkoxy, amino, alkylamino, aryl or arylamino group;
X is hydroxy, carboxy, acetyl, alkylcarbonyl, arylcarbonyl, formyl, C₁-C₆ alkyl substituted with one to six hydroxy groups, aryl, heteroaryl, alkyloxycarbonyl, N-alkylcarbamoyl or —C(═N—R₅)R₆;
R₆ is hydrogen or CH₃;
D is CH, CH₂, CCH₃, CHCH₃, CHCH₂OH, or carbonyl; and
—— is a single or a double bond;
provided that
i.) when A is carbonyl or C═N—R₅ then n=1;
ii.) in compounds of Formula I,
when n=0 and X is aryl, heteroaryl, formyl, carboxy, acetyl, alkyloxycarbonyl, arylcarbonyl, N-alkylcarbamoyl,

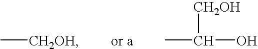

group, then R₁, R₂, R₃ and R₄ are not all hydrogen; and
when n=0, R₁=R₂=R₄═H, and R₃═OH or
when n=0, R₂=R₄═H, and R₁=R₃═OH or
when n=0, R₁=R₄═H, and R₂=R₃═OH or
when n=0, R₁=R₂═H, and R₃═R₄═OH,
then X is not a formyl or 2-formylphenyl; and
when n=0, R₁=R₂=R₄═H, and R₃═OH or
when n=0, R₂=R₄═H, and R₁=R₃═OH,
then X is not 4-carboxyphenyl group; and
when n=0, R₁=R₂═R-4=H, and R₃═OH,
then X is not carboxy, phenyl, 4-styrylphenyl, 4-(N,N-dimethylamino)phenyl, 2-pyridinyl, 3-pyridinyl, 2-naphtalenyl; and
when n=0, R₁=R₂=R₃═H, and R₄=Me
then X is not phenyl, 3-bromophenyl, 2-chlorophenyl, 3-chlorophenyl, 4-chlorophenyl, 2,4-dichlorophenyl, 2-methoxyphenyl, 3-methoxyphenyl, 4-methoxyphenyl, 4-nitrorophenyl, 4-hydroxy-3-methoxyphenyl, 4-(N,N-dimethylamino)phenyl, 4-methylthiophenyl;
when n=0, R₁=R₃=R₄═H, and R₂=Me
then X is not phenyl, 2-chlorophenyl, 4-hydroxyphenyl, 2,4-dichlorophenyl, 3-methoxyphenyl, 4-methoxyphenyl 4-hydroxy-3-methoxyphenyl, 3-nitrorophenyl, 2-nitrorophenyl, 2-methoxyphenyl, 3,4,5-trimethoxyphenyl, 4-(N,N-dimethylamino)phenyl, 2-pyridinyl, 3-pyridinyl, 4-pyridinyl, 2-quinolinyl; and
when n=0, R₁=R₃=R₄═H, and R₂—F or
when n=0, R₁=R₂=R₄═H, and R₃═F
then X is not carboxy, ethyloxycarbonyl,
when n=0, R₁=R₂=R₃═H, and R₄═C1
then X is not phenyl,
when n=0, R₁=R₃═R═H, and R₂═Cl
then X is not phenyl, 4-hydroxyphenyl, 4-methoxyphenyl, 3-nitrophenyl, 4-hydroxy-3-methoxyphenyl, 2-pyridinyl, 3-pyridinyl, 4-pyridinyl, 2-(6-methyl)pyridinyl, 2-quinolinyl,
when n=0, R₁=R₃=R₄═H, and R₂=Br
then X is not phenyl, 2-hydroxyphenyl, 3-hydroxyphenyl, 4-methylphenyl, 4-hydroxy-3-methoxyphenyl, 4-hydroxy-3-ethoxyphenyl, 2-pyridinyl, 4-(N,N-dimethylamino)phenyl, 5-benzo[1,3]dioxolyl; and
when n=0, R₁=R₂=R₄═H, and R₃=Me or
when n=0, R₂=R₃═H and R═R₄=Me,
then X is not phenyl, 4-hydroxyphenyl, 4-nitrophenyl; and
when n=0, R₂=R₄═H and R₁=R₃=Me,
then X is not phenyl; and
when n=0, R₁=R₄═H and R₂═C₁ and R₃=Me,
then X is not phenyl, 4-hydroxyphenyl, 4-methoxyphenyl, 2-(6-methyl)pyridinyl, 3-pyridinyl, 4-pyridinyl, 2-quinolinyl; and
when n=0, R₁=R₄═H and R₂=R₃=Me,
then X is not phenyl, 4-methoxyphenyl; and
when n=0, R₁=R₃═H and R₂=R₄═Cl,
then X is not phenyl
when n=0, R₁=R₂═H and R₃═OH and R₄=Me,
then X is not phenyl, 4-methoxyphenyl, 2-hydroxyphenyl, 3-hydroxyphenyl, 4-hydroxy-3-methoxyphenyl, 4-hydroxy-3-ethoxyphenyl, 5-benzo[1,3]dioxolyl; and
when n=1 and X is carboxy, ethyloxycarbonyl
then R₁, R₂, R₃ and R₄ are not all hydrogen; and
when n=1 and R₁=R₃=R₄═H and R₂=Br,
then X is not phenyl;
   iii.) in compounds of Formula II,
when X is aryl, heteroaryl, carboxy, acetyl, alkyloxycarbonyl,

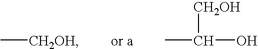

group, then R₁, R₂, R₃ and R₄ are not all hydrogen; and
when R₁=R₃═R₄═H and R₂=Me,
then X is not 3-(4-hydroxy-6-methyl-2-oxo-2H-1-benzopyranyl);
   iv.) in compounds of Formula III,
when D represents CH₂, CHCH₃, CHCH₂OH, or carbonyl and —— represents a single bond, or when D represents CH or CCH₃ group and —— represents a double bond, then R₁, R₂, R₃ and R₄ are simultaneously not hydrogen atom; and
when R₁=R₂=R₄═H and R₃═OH, or
when R₂=R₄═H and R₁=R₃═OH, or
when R₁=R₄═H and R₂=R₃═OH, or
P when R₁=R₂═H, R₃═R₄═OH,
then D is not a CH or CH₂ group.

Some compounds of Formulas I, II and III with unsubstituted benzene rings (R1=R2=R3=R4=H) are known in the literature (Fucik, K. et al. Nature 1950, 166, 830-831; Collect. Czech. Chem. Commun. 1951, 16, 304-318; ibid., 1951, 16, 319-326; Bull. Soc. Chim. Fr. 1949, 16, 99-103; ibid., 1949, 16, 609-610, ibid., 1949, 16, 626-628, by Eckstein, M. et al. Roczniki Chem. 1964, 38, 1115-1120; Acta Pol. Pharm. 1988, 45, 8-13, or by Sullivan, W. R. et. al. J. Am. Chem. Soc. 1943, 65, 2288-2291), several of which compounds are reported to have anticoagulant properties. Some compounds of Formulae I and III with unsubstituted benzene rings (R1=R2=R3=R4=H) and hydroxy substituents on benzene rings (R1=R2=R4=H, R3=OH; R2=R4=H, R1=R3=OH; R1=R4=H, R2=R3=OH; R1=R2=H, R3=R4=OH) are also known from the patent literature (Ivezic, Z. et. al. WO 03/029237), and are reported to possess antiviral activity.

There are also known more complex dimeric and tetrameric derivatives of hydroxycoumarin asymmetrically bound by a central alkyl or aryl linker, which demonstrate anti-HIV action (Zhao, H. et al. J. Med. Chem. 1997, 40, 242-249). Similar anti-HIV action is also shown by several products of condensation of hydroxycoumarins possessing more than one hydroxy group per coumarin unit with aromatic or aliphatic mono- or dialdehydes (U.S. Pat. No. 6,100,409 and WO 03/029237).

Bis-(coumarin) compounds with unsubstituted and variously substituted benzene rings which are represented by Formula I, compounds of Formula II with various R₁, R₂, R₃, and R₄ groups, and compounds of Formula III, wherein the benzene rings are substituted with various R₁ and/or R₂ and/or R₃ and/or R₄ groups such as alkyl and alkoxy groups and halogen atoms, as well as their pharmaceutically acceptable salts and pharmaceutical preparations including such compounds, have not hitherto been described. Likewise, the compounds of the present invention have not hitherto been described as substances with a strong antiinflammatory action or as effective agents in the treatment of asthma and other inflammatory diseases and conditions.

The applied in vitro and in vivo models quite successfully demonstrate pathophysiological occurrences present in asthma and it may be expected that the compounds tested in these models will also be effective in the therapy of human diseases.

### DETAILED DESCRIPTION OF THE INVENTION

Compounds of the Invention

One particular class of compounds of Formula I are those wherein n=1 and A represents a carbonyl group.

In some embodiments, the compounds of the present invention are represented by the compounds of Formula I or a pharmaceutically acceptable salt, solvate, tautomer or stereoisomer thereof,
wherein:
R₁, R₂, R₃ and R₄ is each independently hydrogen, fluoro, chloro, bromo, C₁-C₃-alkyl, hydroxy, or nitro;
A is carbonyl or CH—X,
Each occurrence of n is, independently, an integer which is 0 or 1; and
X is carboxy, —C(O)O(C₁₋₄alkyl), —CH(OH)CH₂OH, —CH2OH, C(O)OCH₂CH2OH, C(O)NHdecyl, C(O)NH(CH₂)₃OH, C(O)NHC(CH₃)₂CH₂OH, C(O)NHC(CH₂OH)₃, 1-hydroxyethyl, —C(═N)—OH, imidazolyl, phenyl, 2-methoxyphenyl, 4-quinolinyl, 2-quinolinyl, 2-nitrophenyl, 3-chlorophenyl, 2,4-dimethoxyphenyl, 4-(1-butyl)-imidazolyl, naphthyl, 2-hydroxy-4-nitrophenyl, 6-(2,3-dihydrobenzodioxanyl), 2-(4-chlorophenylthio)-phenyl, 2-pyridinyl, 3-bromo-4-fluorophenyl, 3-bromo-4-methoxyphenyl, 3-chloro-4-fluorophenyl, 3-ethoxyphenyl, 3-hydroxyphenyl, 3-phenoxyphenyl, 3-methoxy-4-benzyloxyphenyl, 4-thiomethylphenyl, 2-(4-bromothiophenyl), 4-chlorophenyl, 4-cyanophenyl, 4-isopropoxyphenyl, 4-methylphenyl, 4-phenoxyphenyl, 2-(5-methyl)-furanyl, 3,4,5-trimethoxyphenyl, 4-fluorophenyl, 4-pyridyl, 2-chlorophenyl, 1-imidazolyl, 3,4-methylenedioxyphenyl, 4-methoxyphenyl, 3-quinolinyl, 2-quinolinyl, 4-hydroxyphenyl, 5-(2,3-dihydro)-benzofuranyl, 2-methoxyphenyl, 2-furanyl, 4-trifluoromethylphenyl, 2-[5-(3-trifluoromethylphenyl)]-furanyl, 1-(5-hydroxymethyl)-furanyl; and

provided that
i.) when A is carbonyl then n=1; and
ii.) when n=0 and X is carboxy, —C(O)O(C₁₋₄alkyl), —CH(OH)CH₂OH, —CH₂OH, C(O)OCH₂CH2OH, C(O)NHdecyl, C(O)NH(CH₂)₃OH, C(O)NHC(CH₃)₂CH₂OH, C(O)NHC(CH₂OH)₃, imidazolyl, phenyl, 2-methoxyphenyl, 4-quinolinyl, 2-quinolinyl, 2-nitrophenyl, 3-chlorophenyl, 2,4-dimethoxyphenyl, 4-(1-butyl)-imidazolyl, naphthyl, 2-hydroxy-4-nitrophenyl, 6-(2,3-dihydrobenzodioxanyl), 2-(4-chlorophenylthio)-phenyl, 2-pyridinyl, 3-bromo-4-fluorophenyl, 3-bromo-4-methoxyphenyl, 3-chloro-4-fluorophenyl, 3-ethoxyphenyl, 3-hydroxyphenyl, 3-phenoxyphenyl, 3-methoxy-4-benzyloxyphenyl, 4-thiomethylphenyl, 2-(4-bromothiophenyl), 4-chlorophenyl, 4-cyanophenyl, 4-isopropoxyphenyl, 4-methylphenyl, 4-phenoxyphenyl, 2-(5-methyl)-furanyl, 3,4,5-trimethoxyphenyl, 4-fluorophenyl, 4-pyridyl, 2-chlorophenyl, 1-imidazolyl, 3,4-methylenedioxyphenyl, 4-methoxyphenyl, 3-quinolinyl, 2-quinolinyl, 4-hydroxyphenyl, 5-(2,3-dihydro)-benzofuranyl, 2-methoxyphenyl, 2-furanyl, 4-trifluoromethylphenyl, 2-[5-(3-trifluoromethylphenyl)]-furanyl, or 1-(5-hydroxymethyl)-furanyl; then R₁, R₂, R₃ and R₄ are not all hydrogen; and
iii.) when n=0, R₁=R₂=R₄═H, and R₃═OH,
   then X is not carboxy, phenyl, 2-pyridinyl, or 2-naphtalenyl; and
iv.) when n=0, R₁=R₂=R₃═H, and R₄=Me
   then X is not phenyl, 2-chlorophenyl, 3-chlorophenyl, 4-chlorophenyl, 2-methoxyphenyl, 4-methoxyphenyl, or 4-methylthiophenyl; and
v.) when n=0, R₁=R₃═R₄═H, and R₂=Me
   then X is not phenyl, 2-chlorophenyl, 4-hydroxyphenyl, 4-methoxyphenyl, 2-methoxyphenyl, 3,4,5-trimethoxyphenyl, 2-pyridinyl, 4-pyridinyl, or 2-quinolinyl; and
vi.) when n=0, R₁=R₃═R₄═H, and R₂═F or
   when n=0, R₁=R₂=R₄═H, and R₃═F
   then X is not carboxy, or ethyloxycarbonyl; and
vii.) when n=0, R₁=R₂=R₃═H, and R₄═Cl
   then X is not phenyl; and
viii.) when n=0, R₁=R₃=R₄═H, and R₂=Cl
   then X is not phenyl, 4-hydroxyphenyl, 4-methoxyphenyl, 2-pyridinyl, 4-pyridinyl, or 2-quinolinyl; and
ix.) when n=0, R₁=R₃=R₄═H, and R₂=Br
   then X is not phenyl, 3-hydroxyphenyl, 4-methylphenyl, 2-pyridinyl, or 5-benzo[1,3]dioxolyl; and
x.) when n=0, R₁=R₂=R₄═H, and R₃=Me or
   when n=0, R₂=R₃═H and R═R₄=Me,
   then X is not phenyl, or 4-hydroxyphenyl; and
xi.) when n=0, R₂=R₄═H and R₁=R₃=Me,
   then X is not phenyl; and
xii.) when n=0, R₁=R₄═H and R₂═C₁ and R₃=Me,
   then X is not phenyl, 4-hydroxyphenyl, 4-methoxyphenyl, 4-pyridinyl, or 2-quinolinyl; and
xiii.) when n=0, R₁=R₄═H and R₂=R₃=Me,
   then X is not phenyl, or 4-methoxyphenyl; and
xiv.) when n=0, R₁=R₃═H and R₂=R₄═Cl,
   then X is not phenyl; and
xv.) when n=0, R₁=R₂═H and R₃═OH and R₄=Me,
   then X is not phenyl, 4-methoxyphenyl, 3-hydroxyphenyl, or 5-benzo[1,3]dioxolyl; and
xvi.) when n=1 and X is carboxy or ethyloxycarbonyl
   then R₁, R₂, R₃ and R₄ are not all hydrogen; and
xvii.) when n=1 and R₁=R₃═R₄═H and R₂==Br,
   then X is not phenyl.

For the compounds of Formula I, it is further provided that,

(i) when n=0, R₁=R₃=R═H, and R₂=Br
then X is not 4-hydroxyphenyl or 3,4-methylenedioxyphenyl; and
(ii) when n=0, R₁=R₂=R₄═H, and R₃═Cl
then X is not phenyl.

A further particular class of compounds are those of Formula I wherein n=0, X is carboxy, acetyl, alkylcarbonyl, arylcarbonyl, C₁-C₆ alkyl substituted with one to six hydroxy groups, alkyloxycarbonyl, N-alkylcarbamoyl, formyl, or —C(═N—R₅)R₆ and at least one of the R₁, R₂, R₃ and R₄ are each independently, chloro, bromo, C₁-C₄-alkyl, C₂-C₄-alkenyl, C₂-C₄-alkynyl, halo-C₁-C₄-alkyl, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro; most preferably R₁, R₂, R₃ and R₄ are each independently C₁-C₄-alkyl, chloro, bromo.

A still further particular class of compounds are those of Formula I wherein n=0, X is arylcarbonyl, C₁-C₆ alkyl substituted with one to six hydroxy groups, N-alkylcarbamoyl, or —C(═N—R₅)R₆ and at least one of the R₁, R₂, R₃ and R₄ are each independently, fluoro, chloro, bromo, C₁-C₄-alkyl, hydroxy, C₂-C₄-alkenyl, C₂-C₄-alkynyl, halo-C₁-C₄-alkyl, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro; most preferably R₁, R₂, R₃ and R₄ are each independently C₁-C₄-alkyl, fluoro, chloro, bromo or hydroxy.
Another particular class of compounds are those of Formula I wherein n=0, X is 2-1H-imidazolyl, 6-(2,3-dihydro)benzo[1,4]dioxinyl, 3-bromo-4-fluorophenyl, 3-bromo-4-methoxyphenyl, 3-ethoxyphenyl, 3-phenoxyphenyl, 4-phenoxyphenyl, 4-benzyloxy-3-methoxyphenyl, 2-(4-bromo)thiophenyl, 4-isopropoxyphenyl, 3-quinolinyl, 4-quinolinyl, 5-(2,3-dihydro)benzofuranyl, 2-furanyl, 4-trifluoromethylphenyl, 2-[5-(3-trifluoromethylphenyl)]furanyl or 2-(5-hydroxymethyl)furanyl and at least one of the R₁, R₂, R₃ and R₄ are each independently, fluoro, chloro, bromo, C₁-C₄-alkyl, hydroxy, C₂-C₄-alkenyl, C₂-C₄-alkynyl, halo-C₁-C₄-alkyl, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro; most preferably R₁, R₂, R₃ and R₄ are each independently C₁-C₄-alkyl, fluoro, chloro or bromo.

Other representative compounds include:
(a) compounds of formula I, wherein n=0, X is phenyl, R₁=R₂=R₄═H and R₃═Cl;
(b) compounds of formula I, wherein n=0, X is phenyl R₁=R₂=R₃═H and R₄=isopropyl;
(c) compounds of formula I, wherein n=0, X is 4-methylphenyl, 3-hydroxyphenyl, 4-hydroxyphenyl, 3-chlorophenyl, 4-chlorophenyl, 4-methylthiophenyl, 2-pyridinyl, 2-quinolinyl or 5-benzo[1,3]dioxolyl; R₁=R₄═H and R₂=R₃=Me;
(d) compounds of formula I, wherein n=0, X is 4-methylphenyl, 3-hydroxyphenyl, 4-hydroxyphenyl, 4-chlorophenyl, 4-methylthiophenyl, 2-pyridinyl, 3-pyridinyl, 4-pyridinyl, 2-quinolinyl, 4-methoxyphenyl or 5-benzo[1,3]dioxolyl; R₁=R₃═H and R₂=R₄=Me;
(e) compounds of formula I, wherein n=0, X is 4-hydroxyphenyl, 4-methoxyphenyl, 4-chlorophenyl, 4-methylthiophenyl, 2-quinolinyl, 3-pyridinyl or 4-pyridinyl; R₁=R₃═R₄═H and R₂=Et, F or Br;
(f) compounds of formula I, wherein n=0, X is 4-methylphenyl, 2-pyridinyl, 3-hydroxyphenyl or 5-benzo[1,3]dioxolyl; R₁=R₃=R₄═H and R₂=Et or F;
(g) compounds of formula I, wherein n=0, X is 4-methylphenyl, 3-hydroxyphenyl, 3-chlorophenyl, 4-chlorophenyl, 4-methylthiophenyl or 5-benzo[1,3]dioxolyl; R₁=R₃=R₄═H and R₂═Cl;
(h) compounds of formula I, wherein n=0, X is 4-methylphenyl, 3-hydroxyphenyl, 4-chlorophenyl, 4-methylthiophenyl, 2-pyridinyl, 4-pyridinyl, 2-quinolinyl or 5-benzo[1,3]dioxolyl; R₁=R₂=R₄═H and R₃=Me;
(i) compounds of formula I, wherein n=0, X is 4-methylphenyl, 3-hydroxyphenyl, 3-chlorophenyl, 4-chlorophenyl, 4-methylthiophenyl, 2-pyridinyl or 5-benzo[1,3]dioxolyl; R₁=R₄═H; R₂═C₁ and R₃=Me;
(j) compounds of formula I, wherein n=0, X is 5-benzo[1,3]dioxolyl; R₁=R₃=R₄═H and R₂=Me
(k) compounds of formula I, wherein n=0, X is carboxy; R₁=Me; R₂=R₄═H and R₃═OH
(l) compounds of formula I, wherein n=0, X is acetyl; R₁=R₃═OH and R₂=R₄═H

A further particular class of compounds are those of Formula II wherein X is carboxy, acetyl, alkylcarbonyl, arylcarbonyl, formyl, C₁-C₆ alkyl substituted with one to six hydroxy groups, aryl, alkyloxycarbonyl, N-alkylcarbamoyl or —C(═N—R₅)R₆ and at least one of the R₁, R₂, R₃ and R₄ are each independently, fluoro, chloro, bromo, C₁-C₄-alkyl, hydroxy, C₂-C₄-alkenyl, C₂-C₄-alkynyl, halo-C₁-C₄-alkyl, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-allkoxycarbonyl, cyano, or nitro; most preferably R₁, R₂, R₃ and R₄ are each independently C₁-C₄-alkyl, fluoro, chloro or bromo.

A further particular class of compounds are those of Formula II wherein X is carboxy, acetyl, formyl, C₁-C₆ alkyl substituted with one to six hydroxy groups and at least one of the R₁, R₂, R₃ and R₄ are each independently, C₁-C₄-alkyl, fluoro, chloro or bromo.

A further particular class of compounds are those of Formula III wherein

D is CHCH₃, CHCH₂OH or carbonyl and —— is a single bond;
or D is CCH₃ and —— is a double bond;
and at least one of the R₁, R₂, R₃ and R₄ are each independently, fluoro, chloro, bromo, C₁-C₄-alkyl, hydroxy, C₂-C₄-alkenyl, C₂-C₄-alkynyl, halo-C₁-C₄-alkyl, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro; most preferably R₁, R₂, R₃ and R₄ are each independently C₁-C₄-alkyl, fluoro, chloro or bromo or hydroxy.

A further particular class of compounds are those of Formula III wherein

### D is CH or CH₂;

and at least one of the R₁, R₂, R₃ and R₄ are each independently, fluoro, chloro, bromo, C₁-C₄-alkyl, C₂-C₄-alkenyl, C₂-C₄-alkynyl, halo-C₁-C₄-alkyl, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro; most preferably R₁, R₂, R₃ and R₄ are each independently C₁-C₄-alkyl, fluoro, chloro or bromo.

In the context of the present invention the general terms used above have the following meanings:

The term “halogen” relates to a halogen atom, which may be: fluorine, chlorine, bromine or iodine.

The term “alkyl” relates to alkyl groups derived from alkanes, which may be straight, branched or cyclic or a combination of straight and cyclic chains or of branched and cyclic chains. The preferred straight or branched alkyls are e.g. methyl, ethyl, propyl, isopropyl, butyl, sec-butyl and tert-butyl. Methyl is most preferred. The preferred cyclic alkyls are e.g. cyclopentyl or cyclohexyl. Alkyl groups may be substituted with one up to five substituents including halogen (preferably fluorine or chlorine), hydroxy, alkoxy (preferably methoxy or ethoxy), acyl, acylamino cyano, amino, N—(C₁-C₄)alkylamino (preferably N-methylamino or N-ethylamino), N,N-di(C₁-C₄-alkyl)amino (preferably dimethylamino or diethylamino), aryl (preferably phenyl) or heteroaryl, thiocarbonylamino, acyloxy, amino, amidino, alkyl amidino, thioamidino, aminoacyl, aminocarbonylamino, aminothiocarbonylamino, aminocarbonyloxy, aryl, heteroaryl, aryloxy, aryloxyaryl, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, cycloalkoxy, heteroaryloxy, heterocyclyloxy, and oxycarbonylamino. Such substituted alkyl groups are within the present definition of “alkyl.”

The term “C₁-C₄-alkyl” represents an alkyl group with 1 to 4 carbon atoms. Similarly, the term “C₁-C₇-alkyl” represents an alkyl group with 1 to 7 carbon atoms. Similar terminology is used herein to represent the number of carbon atoms within a specified group. The present definition of alkyl carries over to other groups having an alkyl moiety such as alkoxy.

“Alkenyl” means a linear or branched monovalent hydrocarbon radical of two to ten, preferably two to six, carbon atoms which has at least one carbon-carbon double bond. Alkenyl groups may be substituted with the same groups as alkyl and such optionally substituted alkenyl groups are encompassed within the term “alkenyl.” Ethenyl, propenyl, butenyl and cyclohexenyl are preferred.

“Alkynyl” means a linear or branched monovalent hydrocarbon radical, having a straight-chain or a branched-chain of two to ten, preferably two to six, carbon atoms and containing at least one and preferably no more than three carbon-carbon triple bonds. Alkynyl groups can be substituted with the same groups as alkyl, and the substituted groups are within the present definition of alkynyl. Ethynyl, propynyl and butynyl groups are preferred.

“Cycloalkyl” means a cyclic group having 3-8 carbon atoms having a single ring optionally fused to an aryl or heteroaryl group. The cycloalkyl groups can be substituted as specified for “aryl” below, and the substituted cycloalkyl groups are within the present definition of “cycloalkyl”. Preferred cycloalkyls are cyclopentyl and cyclohexyl.

“Aryl” means an unsaturated aromatic carbocyclic group having 6-14 carbon atoms having a single ring such as phenyl or multiple fused rings such as naphthyl. Aryl may optionally be further fused to an aliphatic or aryl group or can be substituted with one or more substituents such as halogen (fluorine, chlorine and/or bromine), hydroxy, C₁-C₇ alkyl, C₁-C₇ alkoxy or aryloxy, C₁-C₇ alkylthio or arylthio, alkylsulfonyl, cyano or primary or nonprimary amino.

“Heteroaryl” means a monocyclic or a bicyclic aromatic hydrocarbon ring having from 2 to 10 carbon atoms and from 1 to 4 heteroatoms, such as O, S or N. The heteroaryl ring may optionally be fused to another heteroaryl, aryl or aliphatic cyclic group. Examples of this type are furan, thiophene, pyrrole, imidazole, indole, pyridine, oxazole, thiazole, pyrrole, pyrazole, tetrazole, pyrimidine, pyrazine and triazine, with furan, pyrrole, pyridine and indole being preferred. The term includes groups that are substituted with the same substituents as specified for aryl above.

“Heterocyclic” means a saturated or unsaturated group having a single or multiple rings and from 1 to 10 carbon atoms and from 1-4 heteroatoms selected from nitrogen, sulphur or oxygen, wherein in a fused ring system the other ring or rings can be aryl or heteroaryl. Heterocyclic groups can be substituted as specified for alkyl groups and the thus substituted heterocyclic groups are within the present definition.

The term “alkoxy” relates to straight or branched chains containing an alkoxy group. Examples of such groups are methoxy, propoxy, prop-2-oxy, butoxy, but-2-oxy or methylprop-2-oxy.

The term “alkanoyl” group relates to straight chains containing an acyl group such as formyl, acetyl or propanoyl.

The term “aroyl” group relates to aromatic acyl groups such as benzoyl.

Compounds of Formulae I and III possess at least one acidic hydroxy group on a coumarin nucleus, as well as the compounds of formula II wherein X denotes a carboxy group. Thus, these compounds may form corresponding salts with pharmaceutically acceptable bases. The present invention encompasses such salts. Examples of salts formed on a hydroxy substituent are e.g. aluminum salts, corresponding salts of alkali metals such as sodium or potassium, salts of alkaline earth metals such as calcium or magnesium, pharmaceutically acceptable salts of transient metals such as zinc and copper, salts with ammonia or salts with lower organic amines such as cyclic amines, mono-, di- or trisubstituted lower alkylamines, lower hydroxyalkylamines such as lower mono-, di- or trihydroxyalkylamines, lower (hydroxyalkyl)alkylamines or lower polyhydroxyalkylamines and salts with amino acids, e.g., methylglutainine, alanine or serine. Suitable pharmaceutically acceptable cyclic amines include, e.g., morpholine, thiomorpholine, piperidine or pyrrolidine. Suitable lower monoalkylamines include, e.g., ethylamine and tert-butylamine, suitable dialkylamines include, e.g., diethylamine and diisopropylamine and suitable lower trialkylamines include, e.g., trimethylamine and triethylamine. Corresponding lower hydroxyalkylamines include, e.g., mono-, di- or triethanolamine; lower (hydroxyalkyl)alkylamines include, e.g., N,N-dimethylaminoethanol and N,N-diethylaminoethanol. Suitable amino acids include, e.g., lysine, arginine, methylglutamine, alanine or serine.

Any substituent having basic properties, e.g, including an amino or alkylamino group, may also form pharmaceutically suitable salts with inorganic acids (e.g. hydrochloric, hydrobromic, phosphoric, metaphosphoric, nitric or sulfuric acid) or organic acids (e.g. tartaric, acetic, methane-sulfonic, trifluoroacetic, citric, maleic, lactic, fumaric, benzoic, succinic, methanesulfonic, oxalic and p-toluenesulfonic acids). These salts may be prepared in situ during the final isolation and purification of the compounds of the present invention or separately in a reaction with a suitable inorganic or organic base in a manner known to one skilled in the art, for example in a suitable solvent or solvent mixture, e.g., in ethers (diethylether) or alcohols (ethanol, n-propanol, 2-propanol or tert-butanol), or by mixing equivalent amounts of corresponding reactants and the subsequent lyophilization and purification of the reaction mixture.

The prefix “lower” designates a radical having up to and including seven and, preferably, up to and including four carbon atoms. Lower alkyl is, for example, n-propyl, isopropyl, n-butyl, isobutyl, sec-butyl, tert-butyl, n-pentyl, neopentyl, n-hexyl or n-heptyl, preferably ethyl or methyl.

In view of the close connection between free forms and salt forms of the compounds of the present invention it should be understood that in the present invention the free forms of the compounds of the present invention and their pharmaceutically acceptable salts are identical forms and in the corresponding context it is suitable to consider the free forms of the compounds of the present invention and their corresponding pharmaceutically acceptable salts as synonymous.

The present invention also encompasses prodrugs of compounds of Formulae I, II and III, i.e. compounds which release an active drug according to Formulae I, II or III in vivo when administered to a mammalian subject. Prodrugs of a compound of Formulae I, II and III are prepared by modifying functional groups present in the compound of Formulae I, II and III in such a way that the modifications may be cleaved in vivo to release the parent compound. Prodrugs include compounds of Formulae I, II and III wherein a hydroxy, amino, or carboxy group of a compound of Formulae I, II and III is bonded to any group that may be cleaved in vivo to regenerate the free hydroxy, amino or carboxy group, respectively. Examples of prodrugs include, but are not limited to esters (e.g., acetate, formate, and benzoate derivatives) of compounds of Formulae I, II and III or any other derivative which upon being brought to the physiological pH or through enzyme action is converted to the active parent drug.

The present invention also relates to solvates (preferably hydrates) that can be formed by the compounds of Formulae I, II and III or their salts. The present invention also relates to clathrates that can be formed by the compounds of Formulae I, II and III or their salts.

The compounds represented by Formulae I, II and III, and their salts may exist in more than one physical form (e.g. in different crystal forms) and the present invention relates to all physical forms (e.g. to all crystal forms) of the compounds represented by Formulae I, II and III, and to their mixtures.

The compounds of Formulae I, II and III may exist in numerous forms of structural isomers that may be formed as a result of tautomerism, and may exist in different ratios at equilibrium. Due to dynamic equilibrium such isomers (tautomers) are rapidly interconvertible from one isomeric form to another. The most common isomerism is keto-enol tautomerism, but equilibrium between open chain and cyclic forms is also known. It is to be understood that whenever in the present invention reference is made to the compounds of Formulae I, II and III it is intended to include tautomeric forms thereof, keto-enol tautomeric, open chain-cyclic, isolated as separate isomers or existing in any other mixture of different ratios at equilibrium. The isomeric forms predominant for a particular compound of Formulae I, II and III are dependent on the nature of the substituent, whether the compound exists in the free form or in the form of any of its salts, type of the salt, solvent in which the compound is dissolved, as well as pH value of the solution.

Compounds of the present invention may further exist as different geometric isomers or as different stereoisomers. Isomers that differ only with regard to the arrangement of the atoms in the space around the asymmetric (stereogenic, chiral) center are called “stereoisomers”. Stereoisomers that are not mirror images of each other are called diastereomers, while stereoisomers that have a mirror-image relationship, i.e. that are mirror images of each other are called enantiomers. Each stercoisomer may be characterized by determining the absolute configuration of the stereogenic center by the use of Cahn-Ingold-Prelog priority rules and hence characterized as R- or S-isomer. Another way of identification of stereoisomers is the measurement of the rotation of the plane of polarized light that passes through the molecule, and designating chiral molecules to be right-rotating (+) or left-rotating (−) isomers. Chiral molecules may exist in a form of single enantiomer or in a mixture of enantiomers. A mixture consisting of equal parts (+) and (−) enantiomers of a chiral substance is called a racemic mixture. The present invention relates to each stereoisomer that may be shown by Formulae I, II and III either isolated as separate enantiomers, diastereomers or existing in racemic or any other mixture thereof. Methods for determination of stereochemical configuration, resolution and separation of stereoisomers are well known in the literature. The enantiomers may be resolved by methods known to those skilled in the art, for example by formation of diastereomeric salts which may be separated, for example, by crystallization; formation of diastereomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. The diastereomeric pairs may be separated by methods known to those skilled in the art, for example chromatography or crystallization and the individual enantiomers within each pair may be separated as described above.

The present invention also encompasses stereoisomers of the syn-anti type, and mixtures thereof encountered when an oxime or similar group is present. The group of highest Cahn-Ingold-Prelog priority attached to one of the terminal doubly bonded atoms of the oxime, is compared with the hydroxy group of the oxime. The stereoisomer is designated as Z (zusammen=together) or Syn if the oxime hydroxyl lies on the same side of a reference plane passing through the C═N double bond as the group of highest priority; the other stereoisomer is designated as E (entgegen=opposite) or Anti.

A further aspect of the present invention involves to the processes for the preparation of the compounds of Formulae I, II and III, and salts thereof and/or, optionally, converting the resulting free compounds represented by Formulae I, II and III, having salt-forming properties into corresponding salts, and/or, optionally, converting the resulting salts into free compounds or into other salts.

The present invention also relates to reactive intermediates obtained during the preparation of the compounds of the present invention and of their pharmaceutically acceptable salts. Such intermediates can be isolated and defined or used without isolation in the next step of chemical synthesis.

It will be appreciated by those skilled in the art that it may be desirable to use protected derivatives of intermediates used in the preparation of the compounds of Formulae I, II and III. Protection and deprotection of functional groups may be performed by methods known in the art. Hydroxy or amino groups may be protected with any hydroxy or amino protecting group, for example, as described in Green, T. W.; Wuts, P. G. M. Protective Groups in Organic Synthesis: John Wiley and Sons, New York, 1999. Selection of protective groups, processes for their addition and removal are common and well known to those skilled in the art. The amino protecting groups may be removed by conventional techniques. For example, acyl groups, such as alkanoyl, alkoxycarbonyl and aroyl groups, may be removed by solvolysis, e.g., by hydrolysis under acidic or basic conditions. Arylmethoxycarbonyl groups (e.g., benzyloxycarbonyl) may be cleaved by hydrogenolysis in the presence of a catalyst such as palladium-on-charcoal.

Synthetic Procedures:

Compounds within Formulae I, II and III can be prepared by following the methods described herein.

Compounds of Formula I wherein A represents a CH—X group, wherein X represents carboxy, acetyl, alkylcarbonyl, —CH₂OH, or an

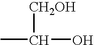

group and n=0, as well as compounds of Formula I wherein A represents a carbonyl group and n=1, may be prepared by a process which comprises condensation of the appropriate hydroxycoumarin of Formula IV:

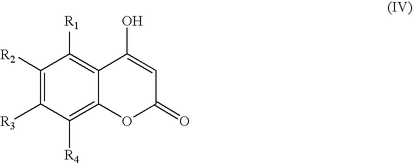

with a corresponding aliphatic aldehyde such as glyoxylic acid, pyruvic aldehyde, glycolaldehyde or glyceraldehyde. Preferably the reaction is carried out in an aqueous medium such as water or aqueous buffer, or aqueous-organic medium inert to the utilized chemical reagents. Suitable organic solvents include, but are not limited to tetrahydrofuran, N,N-dimethylformamide, dimethyl sulfoxide, acetonitrile, acetone, methanol, ethanol, 2-propanol and tert-butanol. Preferable organic solvents include, but are not limited to, acetonitrile, methanol, ethanol and 2-propanol. When buffered aqueous or aqueous-organic medium is used, reaction may be performed in a pH range from 2 to 12, preferably in a pH range from 6 to 9. Commercially available aqueous aldehyde solution may be employed in the reaction, which may be used in substantial excess in respect to hydroxycoumarin, preferably in the ratio 2:1. Reaction may be carried out at a temperature in the range of 0° C. to the boiling point of the solvent employed.

Compounds of Formula I wherein A represents a CH—X group, wherein X represents arylcarbonyl group and n=0, may be prepared by a process which comprises condensation of the appropriate hydroxycoumarin of Formula IV:

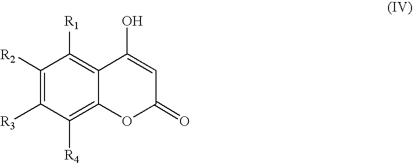

with an aldehyde prepared in situ by oxidation of corresponding α-acyl halide in the presence of DMSO at a temperature in the range of 0° C. to 120° C., preferably at 80° C. (See, e.g., Trkovnik, M. et al. Acta Pharm. Jugosl. 1982, 32, 21-27).

Compounds of Formula I wherein A represents a CH—X group, wherein X denotes CH₂OH and n=0, may optionally be prepared from corresponding intramolecular hemiacetals of Formula III wherein D represents CHOH and —— denotes a single bond (See, e.g., WO 2005/010006) or aldehydes of Formula I wherein A represents a CH—X group, wherein X denotes a formyl group and n=0 (See, e.g. WO 03/029237). Compounds mentioned in this section can be prepared using standard reducing reagents known to those skilled in the art, for example complex metal hydrides such as sodium cyanoborohydride.

Compounds of Formula I wherein A represents a CH—X group, wherein X represents a

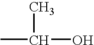

group and n=0, may optionally be prepared from corresponding ketones of Formula I wherein A represents CH—X, wherein X represents an acetyl group and n=0. Compounds mentioned in this section can be prepared using standard reducing reagents known to those skilled in the art, for example complex metal hydrides such as sodium cyanoborohydride.

Compounds of Formula I wherein A represents a CH—X group, wherein X denotes OH and n=1, may optionally be prepared from corresponding ketones of Formula I wherein A represents a carbonyl group and n=1. Compounds mentioned in this section can be prepared using standard reducing reagents known to those skilled in the art, for example complex metal hydrides such as sodium cyanoborohydride.

Compounds of Formula I wherein A represents CH₂ and n=1, may be prepared by a process which comprises the reduction of the compound of Formula I wherein A represents a carbonyl group and n=1, by procedures known to those skilled in the art, for example by catalytic hydrogenation.

Compounds of Formula I wherein A represents a CH—X group, wherein X represents an alkyloxycarbonyl group and n=0, may be prepared by a process which comprises condensation of the appropriate carboxylic acid or lactone with corresponding alcohol with or without the presence of dehydrating agent or acidic catalyst such as sulfuric acid (See, e.g., Fucik, K. et al. Bull. Soc. Chim. Fr. 1949, 16, 99-103; ibid., 1949, 16, 609-610).

Compounds of Formula I wherein A represents a CH—X group, wherein X denotes an N-alkylcarbamoyl group and n=0, may be prepared by a process which comprises condensation of the appropriate carboxylic acid or lactone with corresponding primary amine using standard amide coupling procedures well known to those skilled in the art (See, e.g., Romo, D. et al. J. Am. Chem. Soc. 1998, 120, 12237-12254).

Compounds of Formula I wherein A represents a CH—X group, wherein X represents a —C(═N—R₅)R₆ group, n=0, may be prepared by a process which comprises condensation of the corresponding intramolecular hemiacetal of Formula III wherein D represents CHOH and —— denotes a single bond (See, e.g., WO 2005/010006), or aldehyde of Formula I wherein A represents a CH—X group, wherein X denotes a formyl group and n=0 (See, e.g., WO 03/029237), or ketone of Formula I wherein A represents a CH—X group, wherein X denotes an acetyl or alkylcarbonyl group and n=0, respectively, with hydroxylamine, O-alkylhydroxylamine (e.g. O-methylhydroxylamine), hydrazine, alkylhydrazine (e.g. methylhydrazine), aryl amine (e.g. aniline), and arylhydrazine (e.g. phenylhydrazine), respectively, using standard procedures for the preparation of oximes, imines, and hydrazones well known to those skilled in the art (See, e.g., Hadjipavlou-Litina, D. J. et. al. Bioorg. Med. Chem. Lett. 2004, 14, 611-614).

Compounds of Formula I wherein A represents a C═N—R₅ group, n=1, may be prepared by a process which comprises condensation of the corresponding ketone of Formula I wherein A represents a carbonyl group and n=1, with hydroxylamine, O-alkylhydroxylamine (e.g. O-methylhydroxylamine), hydrazine, alkylhydrazine (e.g. methylhydrazine), aryl amine (e.g. aniline), and arylhydrazine (e.g. phenylhydrazine), respectively, using standard procedures for the preparation of oximes, imines, and hydrazones well known to those skilled in the art (See, e.g., Hadjipavlou-Litina, D. J. et. al. Bioorg. Med. Chem. Lett. 2004, 14, 611-614).

Compounds of Formula I wherein A represents a CH—X group, wherein X is aryl or heteroaryl and n=0 may be prepared by a process which comprises condensation of the appropriate hydroxycoumarin with aryl or heteroaryl aldehyde using procedures known to those skilled in the art (See, e.g., Zhao, H. et al. J. Med. Chem. 1997, 40, 242-249; Sullivan, W. R. et. al. J. Am. Chem. Soc. 1943, 65, 2288-2291).

Compounds of Formula II may be prepared from the corresponding compounds of Formula I wherein n=0, by the process of intramolecular removal of water in which pyran ring is formed using various dehydrating agents such as thionyl chloride, acetic anhydride, acetic anhydride-acetic acid or acetic anhydride-pyridine mixtures (See, e.g., Huebner, C. F. et al. J. Am. Chem. Soc. 1943, 65, 2292-2296; Fucik, K. et al. Collect. Czech. Chem. Commun. 1951, 16, 304-318; ibid., 319-326), following the usual procedures for protection of sensitive functional groups where appropriate. The reaction may be carried out at a temperature in the range of about 0° C. to the boiling point of the employed solvent. In some cases it would be desirable to remove one or more acetyl groups after dehydration process by procedures well known to those skilled in the art, for example by acidic or alkaline hydrolysis.

Compounds of Formula II wherein X represents a formyl group, may optionally be prepared from corresponding intramolecular hemiacetals of Formula III wherein D represents CHOH and —— denotes a single bond (See, e.g., WO 2005/010006), by the process described in the immediately preceding section.

Compounds of Formula II wherein X represents a

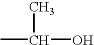

group, may be prepared from corresponding ketones of Formula II wherein X represents an acetyl group. Compounds mentioned in this section can be prepared using standard reducing reagents known to those skilled in the art, for example complex metal hydrides such as sodium cyanoborohydride.

Compounds of Formula III wherein D represents carbonyl group may be prepared from the compounds of Formula I wherein A represents CH—X, wherein X is a carboxy group and n=0, by a process which comprises a formation of lactone ring by procedures known to those skilled in the art (See, e.g., Fucik, K. et al. Bull. Soc. Chem. Fr. 1949, 16, 609-610). Preferably the reaction is carried out in a dehydrating agent, for example thionyl chloride, alkyl monocarboxylic acid such as acetic acid, acetic anhydride-acetic acid or thionyl chloride-acetic acid mixtures. Reactions may be carried out at a temperature in the range of about 0° C. to the about boiling point of the employed solvent. Compounds of Formula III wherein D represents CCH₃ and —— represents a double bond may be prepared from the compounds of Formula I wherein A represents a CH—X group, wherein X is a carboxy group and n=0, by a process which comprises a formation of a furan ring (See, e.g., Fucik, K. et al. Collect. Czech. Chem. Commun, 1951, 16, 296-303; ibid., 304-318). Preferably the reaction is carried out in acetic anhydride or in acetic acid in the presence of dehydrating agent such as sulfuric acid. In some cases it would be desirable to remove one or more acetyl groups after dehydration process by procedures well known to those skilled in the art, for example by acidic or alkaline hydrolysis. Alternatively, the above-mentioned compounds may be prepared from the compounds of Formula I wherein A represents CH—X, wherein X is an acetyl group and n=0, in a dehydrating agent, for example thionyl chloride (See, e.g., Fucik, K. et al. Collect. Czech. Chem. Commun. 1951, 16, 319-326) or trifluoroacetic acid.

Compounds of Formula III wherein D represents a CH₂ group and —— represents a single bond may be prepared by a process which comprises condensation of the appropriate hydroxycoumarin of formula IV with 2-chloroacetaldehyde, its acetal, glycolaldehyde or its acetal (Fucik, K. et al. Bull. Soc. Chim. Fr. 1949, 16, 626-628). Preferably the reaction is carried out in water, alkyl monocarboxylic acid such as acetic acid or trifluoroacetic acid or mixtures thereof followed by usual purification procedures well known to those skilled in the art, for example recrystallization or trituration. Alternatively, the above-mentioned compounds may be prepared from the compounds of Formula I wherein A represents CH—X group, wherein X is —CH₂OH group, n=0, in a dehydrating agent such as trifluoroacetic acid.

Compounds of Formula III wherein D represents a CH group and —— represents a double bond may be prepared by a process which comprises aromatization of the corresponding compound of formula III wherein D represents CH₂ group and —— represents a single bond, and which is the object of the present invention. The aromatization process may be performed by halogenation, most preferably bromination with reagent such as N-bromosuccinimide, in the presence of a radical source such as benzoyl peroxide (Furniss, B. S. et al., Eds., Vogel's Textbook of Practical Organic Chemistry: Longman, London, 1989), followed by in situ dehydrohalogenation. Preferably the reaction is carried out in an organic solvent inert to utilized chemical reagents such as tetrachloromethane. Reaction may be carried out at a temperature in the range of 0° C. to the boiling point, preferably at the boiling point of the employed solvent.

Compounds of Formula III wherein D represents CHCH₂OH and —— represents a single bond may be prepared by a process which comprises condensation of the appropriate hydroxycoumarin of formula IV with glyceraldehyde (See, e.g., Eckstein, M. et al. Roczniki Chem. 1964, 38, 1115-1120). Preferably the reaction is carried out in water, alkyl monocarboxylic acid such as acetic acid or trifluoroacetic acid or mixtures thereof followed by usual purification procedures well known to those skilled in the art, for example recrystallization or trituration. Alternatively, the above-mentioned compounds may be prepared from the compounds of Formula I wherein A represents a CH—X group, wherein X is a

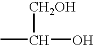

group, in an alkyl monocarboxylic acid such as acetic acid. Reaction may be carried out at a temperature in the range of 0° C. to the boiling point of the employed solvent. In some cases it would be desirable to remove one or more acetyl groups after dehydration process by procedures well known to those skilled in the art, for example by hydrolysis, most preferably by addition of water without isolation of intermediates.

Compounds of Formula III wherein D represents CHCH₃ and —— represents a single bond may be prepared by a process which comprises condensation of the appropriate hydroxycoumarin of formula IV with acrolein or 2-bromo-propionaldehyde (See, e.g., Eckstein, M. et al. Acta Pol. Pharm. 1988, 45, 8-13). Preferably the reaction is carried out in ethanol followed by usual purification procedures well known to those skilled in the art, for example recrystallization or trituration. Reaction may be carried out preferably at the boiling point of the employed solvent. Alternatively, the above-mentioned compounds may be prepared from the compounds of Formula I wherein A represents CH—X group, wherein X is

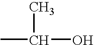

group, n=0, in a dehydrating agent such as trifluoroacetic acid.

Some reagents used in the synthesis of compounds of the present invention are commercial products or they are products previously synthesized and described, while others are obtained according to the processes described for analogous compounds. Thus, for example, hydroxycoumarin compounds of Formula IV may be prepared from the corresponding enamines of Formula V:

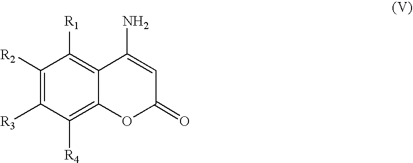

in a manner understandable per se to the one skilled in preparative organic chemistry or known from the literature, e.g. by hydrolysis in a strong acidic medium such as 50% aqueous solution of sulfuric (See, e.g., Desai, N. J. et al. J. Org. Chem. 1957, 22, 388-390) or 25% aqueous solution of hydrochloric acid (See, e.g., Sonn, A. Ber. 1917, 50, 1292-1305). Enamines of Formula V may be prepared in a manner well described in the literature, e.g. by condensation of commercially available phenols with either cyanoacetic acid (See, e.g., Sonn, A. Ber. 1917, 50, 1292-1305) or with its allyl esters such as ethyl cyanoacetate (See, e.g., Desai, N. J. et al., J. Org. Chem. 1957, 22, 388-390). Other methods for the preparation of different derivatives of hydroxycoumarin have previously been described in much detail, such as a direct method of the action of malonic acid on substituted phenols (See, e.g., Buckle, D. et al., J. Med. Chem. 1975, 18, 391-394) or more indirect methods starting from substituted o-hydroxyacetophenones (See, e.g., Boyd, J. et al., J. Chem. Soc. 1948, 174-176; Hermodson, M. et al., J. Med. Chem. 1971, 14, 167-169) or hydroxybenzoic acids (Appendino, G. et al., J. Nat. Prod. 1999, 62, 1627-1631; EP 0694257 A1).

Methods of Use

The present invention relates to the use of the compounds of Formulae I, II and III, and their pharmaceutically acceptable salts, solvates (including hydrates), clathrates, tautomers and stereoisomers in the treatment and prophylaxis of diseases, states, disorders and/or conditions which may occur as a result of disturbance of the immunological system, particularly inflammatory diseases, states, disorders and conditions, especially asthma in mammals (especially humans), in therapeutically effective amounts.

The present invention further relates to the use of the compounds of Formula VI,

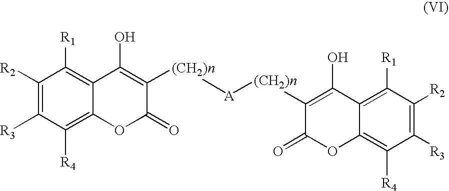

as well as the products of their intramolecular cyclization having Formulas VII and VIII,

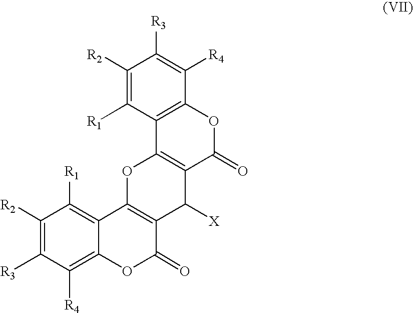

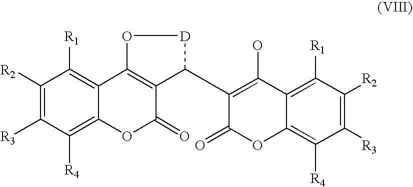

wherein:
R₁, R₂, R₃ and R₄, are each independently hydrogen, halogen, C₁-C₄-alkyl, C₂-C₄-alkenyl, C₂-C₄-alkynyl, halo-C₁-C₄-allyl, hydroxy, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro;
A is carbonyl, CH—X or C═N—R₅;
Each occurrence of n is, independently, an integer which is 0 or 1;
R₅ is an hydroxy, alkoxy, amino, alkylamino, aryl or arylamino group;
X is hydrogen, hydroxy, carboxy, acetyl, alkylcarbonyl, arylcarbonyl, formyl, C₁-C₆ alkyl substituted with one to six hydroxy groups, aryl, heteroaryl, alkyloxycarbonyl, N-alkylcarbamoyl, or —C(═N—R₅)R₆;
R₆ is hydrogen or CH₃;
D is CH, CH₂, CCH₃, CHCH₃, CHCH₂OH, or carbonyl; and
—— is a single or a double bond;
and their pharmaceutically acceptable salts, solvates (including hydrates), clathrates, tautomers and stereoisomers in the treatment and prophylaxis of diseases, states, disorders and/or conditions which may occur as a result of disturbance of the immunological system, particularly inflammatory diseases, states, disorders and conditions, especially asthma in mammals (especially humans), in therapeutically effective amounts

A “therapeutically effective amount” means the amount of a compound that, when administered to a mammal for treating a disease, state, disorder or condition, is sufficient to effect such treatment. The “therapeutically effective amount” will vary depending on the compound, the disease and its severity and the age, weight, physical condition and responsiveness of the mammal to be treated.

“Treating” or “treatment” of a disease, state, disorder or condition includes:

(1) preventing or delaying the appearance of clinical symptoms of the disease, state, disorder or condition developing in a mammal that may be afflicted with or predisposed to the state, disorder or condition but does not yet experience or display clinical or subclinical symptoms of the state, disorder or condition,

(2) inhibiting the state, disorder or condition, i.e., arresting or reducing the development of the disease or at least one clinical or subclinical symptom thereof, or

(3) relieving or attenuating the disease, i.e., causing regression of the state, disorder or condition or at least one of its clinical or subclinical symptoms.

The benefit to a subject to be treated is either statistically significant or at least perceptible to the patient or to the physician.

The four classic symptoms of acute inflammation are redness, elevated temperature, swelling and pain in the affected area, and loss of function of the affected organ. Symptoms and signs; of inflammation associated with specific conditions include:

rheumatoid arthritis—pain, swelling, warmth and tenderness of the involved joints; generalized and morning stiffness;

insulin-dependent diabetes mellitus—insulitis; this condition can lead to a variety of complications with an inflammatory component, including: retinopathy, neuropathy, nephropathy; coronary artery disease, peripheral vascular disease, and cerebrovascular disease;

autoimmune thyroiditis—weakness, constipation, shortness of breath, puffiness of the face, hands and feet, peripheral edema, bradycardia;

multiple sclerosis—spasticity, blurry vision, vertigo, limb weakness, paresthesias;

uveoretinitis—decreased night vision, loss of peripheral vision;

lupus etythematosus—joint pain, rash, photosensitivity, fever, muscle pain, puffiness of the hands and feet, abnormal urinalysis (hematuria, cylinduria, proteinuria), glomerulonephritis, cognitive dysfunction, vessel thrombosis, pericarditis;

scleroderma—Raynaud's disease; swelling of the hands, arms, legs and face; skin thickening; pain, swelling and stuffiness of the fingers and knees, gastrointestinal dysfunction, restrictive lung disease; pericarditis; renal failure;

other arthritic conditions having an inflammatory component such as rheumatoid spondylitis, osteoarthritis, septic arthritis and polyarthritis—fever, pain, swelling, tenderness;

other inflammatory brain disorders, such as meningitis, Alzheimer's disease, AIDS dementia encephalitis—photophobia, cognitive dysfunction, memory loss;

other inflammatory eye inflammations, such as retinitis—decreased visual acuity;

inflammatory skin disorders, such as, eczema, other dermatites (e.g., atopic, contact), psoriasis, burns induced by UV radiation (sun rays and similar UV sources)—erythema, pain, scaling, swelling, tenderness;

inflammatory bowel disease, such as Crohn's disease, ulcerative colitis—pain, diarrhea, constipation, rectal bleeding, fever, arthritis;

asthma—shortness of breath, wheezing;

other allergy disorders, such as allergic rhinitis—sneezing, itching, runny nose conditions associated with acute trauma such as cerebral injury following stroke-sensory loss, motor loss, cognitive loss;

heart tissue injury due to myocardial ischemia—pain, shortness of breath;

lung injury such as that which occurs in adult respiratory distress syndrome—shortness of breath, hyperventilation, decreased oxygenation, pulmonary infiltrates;

inflammation accompanying infection, such as sepsis, septic shock, toxic shock syndrome—fever, respiratory failure, tachycardia, hypotension, leukocytosis;

other inflammatory conditions associated with particular organs or tissues, such as nephritis (e.g., glomerulonephritis)-oliguria, abnormal urinalysis;

inflamed appendix—fever, pain, tenderness, leukocytosis;

gout—pain, tenderness, swelling and erythema of the involved joint, elevated serum and/or urinary uric acid;

inflamed gall bladder—abdominal pain and tenderness, fever, nausea, leulocytosis;

chronic obstructive pulmonary disease—shortness of breath, wheezing;

congestive heart failure—shortness of breath, rates, peripheral edema;

Type II diabetes—end organ complications including cardiovascular, ocular, renal, and peripheral vascular disease

lung fibrosis—hyperventilation, shortness of breath, decreased oxygenation;

vascular disease, such as atherosclerosis and restenosis—pain, loss of sensation, diminished pulses, loss of function; and

alloimmunity leading to transplant rejection—pain, tenderness, fever.

Subclinical symptoms include without limitation diagnostic markers for inflammation the appearance of which may precede the manifestation of clinical symptoms. One class of subclinical symptoms is immunological symptoms including, but not limited to, the invasion or accumulation in an organ or tissue of proinflammatory lymphoid cells or the presence locally or peripherally of activated pro-inflammatory lymphoid cells recognizing a pathogen or an antigen specific to the organ or tissue. Activation of lymphoid cells can be measured by techniques known in the art. Other classes of immunological symptoms are discussed infra in connection with the Examples under the Section “Pharmacological Properties”.

“Delivering” a therapeutically effective amount of an active ingredient to a particular location within a host means causing a therapeutically effective blood concentration of the active ingredient at the particular location. This can be accomplished, e.g., by local or by systemic administration of the active ingredient to the host.

The present invention also includes pharmaceutical compositions containing a therapeutically effective amount of one or more of the compounds of Formula I, II, III, VI, VII, or VIII or a pharmaceutically acceptable salt, solvate, clathrate, tautomer or stereoisomer thereof with a pharmaceutically acceptable diluent or carrier. In a preferred embodiment, the pharmaceutical compositions of the invention are formulated in such a manner to achieve an optimal bioavailability of the active compounds.

As used hereinafter, the term “active compound” denotes the compounds of Formula I, II, III, VI, VII, or VIII or a pharmaceutically acceptable salt, solvate, clathrate, tautomer or stereoisomer thereof.

In therapeutic use, the active compound may be administered orally, buccally, rectally, parenterally, or topically, such as nasally or by inhalation when preferred administration is local application in the respiratory tract. Thus the therapeutic compositions of the present invention may take the form of any of the known pharmaceutical compositions for oral, rectal, parenteral or topical administration. Pharmaceutically acceptable carriers suitable for use in such compositions are well known in the art of pharmacy. The compositions of the invention may contain 0.1-99% by weight of active compound. The compositions of the invention are generally prepared in unit dosage form. Preferably, the unit dosage of active ingredient is 1-500 mg. The effective amount of a compound or a pharmaceutically acceptable salt, solvate, hydrate, clathrate, prodrug, tautomer or stereoisomer thereof is from about 0.004 to about 4000 μmol/kg body weight/day; preferrably from about 0.04 to about 400 μmol/kg body weight/day; more preferrably from about 4 to about 400 μmol/kg body weight/day; most preferrably from about 12 to about 120 μmol/kg body weight/day.

The excipients used in the preparation of these compositions include, but are not limited to the excipients readily known in the pharmacist's art.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

Compositions for oral administration are the known pharmaceutical forms for such administration, for example tablets, capsules, syrups and aqueous or oil suspensions. The excipients used in the preparation of these compositions include, but are not limited to the excipients readily known in the pharmacist's art.

Tablets may be prepared by mixing the active compound with an inert diluent such as calcium phosphate in the presence of disintegrating agents, for example maize starch, and lubricating agents, for example magnesium stearate, and tableting the mixture by known methods. The tablets may be formulated in a manner known to those skilled in the art so as to give a sustained release of the compounds of the present invention. Such tablets may, if desired, be provided with enteric coatings by known methods, for example by the use of cellulose acetate phthalate. Similarly, capsules, for example hard or soft gelatin capsules, containing the active compound with or without added excipients, may be prepared by conventional means and, if desired, provided with enteric coatings in a known manner. The tablets and capsules may conveniently each contain 1 to 500 mg of the active compound. Other compositions for oral administration include, but are not limited to, aqueous suspensions containing the active compound in an aqueous medium in the presence of a non-toxic suspending agent such as sodium carboxymethylcellulose, and oily suspensions containing a compound of the present invention in a suitable vegetable oil, for example arachis oil.

The active compound may be formulated into granules with or without additional excipients. The granules may be ingested directly by the patient or they may be added to a suitable liquid carrier (for example water) before ingestion. The granules may contain disintegrants (for example a pharmaceutically acceptable effervescent couple formed from an acid and a carbonate or bicarbonate salt) to facilitate dispersion in the liquid medium.

Compositions of the invention suitable for rectal administration are the known pharmaceutical forms for such administration, for example, suppositories with cocoa butter or polyethylene glycol bases.

Pharmaceutical compositions may also be administered parenterally (for example subcutaneously, intramuscularly, intradermally and/or intravenously [such as by injection and/or infusion]) in the known pharmaceutical dosage forms for parenteral administration (for example sterile suspensions in aqueous and/or oily media and/or sterile solutions in suitable solvents, preferably isotonic with the blood of the intended patient). Parenteral dosage forms may be sterilized (for example by micro-filtration and/or using suitable sterilising agents [such as ethylene oxide]). Optionally one or more of the following pharmaceutically acceptable adjuvants suitable for parenteral administration may be added to parenteral dosage forms: local anaesthetics, preservatives, buffering agents and/or mixtures thereof. Parenteral dosage forms may be stored in suitable sterile sealed containers (for example ampoules and/or vials) until use. To enhance stability during storage the parenteral dosage form may be frozen after filling the container and fluid (for example water) may be removed under reduced pressure.

Especially important pharmaceutical compositions are those that may be administered nasally or by inhalation in known pharmaceutical forms for such administrations (for example sprays, aerosols, nebulised solutions and/or dry powders). Metered close systems known to those skilled in the art (for example aerosols and/or inhalers) may be used. For all pharmaceutical forms intended for topical administration in respiratory tract it may be beneficial to micronizing the compounds of Formula I, II, III, VI, VII, or VIII or their salts being previously homogenized in lactose, glucose, higher fatty acids, sodium salt of dioctylsulfosuccinic acid, or most preferably in carboxymethylcellulose, in such a way that most of the particles are 5 μm in size. The compounds of the present invention in the form of particles of very small size may be obtained, for example by fluid energy milling. For the inhalation formulation the aerosol can be mixed with a propellant intended for the spraying of the active substance.

Pharmaceutical compositions may be administered to the buccal cavity (for example sub-lingually) in known pharmaceutical forms for such administration (for example slow dissolving tablets, chewing gums, gums, troches, lozenges, pastilles, gels, pastes, mouthwashes, rinses and/or powders).

Compositions for topical administration may comprise a matrix in which the pharmacologically active compound of the present invention is dispersed so that the compound is held in contact with the skin in order to administer the compound transdermally. A suitable transdermal composition may be prepared by mixing the pharmaceutically active compound with a topical vehicle, such as a mineral oil, petrolatum and/or wax, for example paraffin wax or beeswax, together with a potential transdermal accelerant such as dimethyl sulphoxide or propylene glycol. Alternatively the active compound may be dispersed in a pharmaceutically acceptable ointment, cream, gel or lotion. Ointments, creams and gels may be formulated with a water base or an oil base under the addition of a suitable emulsifier or gelling agent when gel is formulated The amount of the active compound contained in a topical formulation should be such that a therapeutically effective amount of the compound is delivered during the period of time for which the topical formulation is intended to be on the skin.

The compounds of the present invention may also be administered by continuous infusion either from an external source, for example by intravenous infusion or from a source of the compound placed within the body. Internal sources include implanted reservoirs containing the compound to be infused which is continuously released for example by osmosis and implants which may be (a) liquid such as a suspension or solution in a pharmaceutically acceptable oil of the compound to be infused for example in the form of s very sparingly water-soluble derivative or (b) solid in the form of an implanted support, for example of a synthetic resin or waxy material, for the compound to be infused. The support may be a single body containing the entire compound or a series of several bodies each containing part of the compound to be delivered. The amount of active compound present in an internal source should be such that a therapeutically effective amount of the compound is delivered over a long period of time.

In the compositions of the present invention the active compound may be used individually or, if desired, may be associated with other compatible pharmacologically active ingredients.

A further aspect of the present invention relates to the use of one or more of the compounds of Formula I, II, III, VI, VII, or VIII, or pharmaceutically acceptable salts, solvates (including hydrates), clathrates, prodrugs, tautomers or stereoisomers thereof or pharmaceutical compositions containing a therapeutically effective amount thereof in the prophylaxis and therapeutic treatment of inflammatory diseases, pathological allergy disorders and/or conditions. Examples of such conditions and diseases are, without limitation, asthma; chronic obstructive pulmonary disease; bronchitis; adult respiratory distress syndrome; nasal inflammatory diseases such as allergic rhinitis, nasal polyps; inflammatatory skin disorders such as eczema, psoriasis, allergic dermatitis, neurodermatitis, pruritis, conjunctivitis; rheumatoid arthritis; inflammatory bowel diseases such as Crohn's disease, colitis and ulcerative colitis; further insulin-dependent diabetes, autoimmune thyroiditis, lupus erythematosus, multiple sclerosis, Raynaud's disease, and other arthritic conditions having an inflammatory component such as rheumatoid spondylitis, septic arthritis, polyarthritis, retinitis, inflammatory brain disorders such as meningitis and encephalitis; conditions associated with acute trauma such as cerebral injury, heart tissue injury and lung injury; inflammation accompanying infections such as sepsis and nephritis (e.g. glomerulonephritis).

### EXAMPLES

The present invention is illustrated by means of the following Examples. The reference to these and other examples anywhere in the specification is illustrative only, and in no way limits the scope and meaning of the invention or of any exemplified form. Likewise, the invention is not limited to any particular preferred embodiments described herein. Indeed, modifications and variations of the invention may be apparent to those skilled in the art upon reading this specification, and can be made without departing from its spirit and scope. The invention is therefore to be limited only by the terms of the appended claims, along with the full scope of equivalents to which the claims are entitled.

All references cited and discussed in this specification are incorporated herein by reference in their entirety and to the same extent as if each reference was individually incorporated by reference.

Processes of Preparation

The preparation processes were mostly carried at atmospheric pressure. In each example the final product was characterised by means of one or several of the following methods: high-performance liquid chromatography (HPLC) and/or high-performance liquid chromatography connected to a mass spectrometer (HPLC-MS) using ESI (electrospray ionisation) and spectroscopy of nuclear magnetic resonance (NMR). Temperatures were expressed in Celsius degrees and the reaction time in hours (h), aq.=aqueous, DMSO=dimethylsulfoxide, DMF=N,N-dimethylformamide, MeCN=acetonitrile, MeOH=methanol, EtOH=ethanol, BuOH=butanol, i-PrOH=2-propanol, Me₂CO=acetone, ether=diethyl ether, AcOEt=ethyl acetate, Py=pyridine, AcOH=acetic acid, Ac₂O=acetic anhydride, TFAA=trifluoroacetic acid, EDC=1-ethyl-3-(3-dimethylaminopropyl)carbodiimide hydrochloride, HOBT=1-hydroxybenzotriazole.

### Examples from 1 to 14 and 290

### General Procedure for the Preparation of Compounds of Formula I

wherein A represents a CH—X group, X is carboxy group and n=0

To a suspension of corresponding 4-hydroxycoumarin (1 equiv.) in MeCN was added 50 wt. % aq. glyoxylic acid (4 equiv.). The reaction mixture was heated to reflux, and stirred under reflux until completion of the reaction (0.5-6 h). After cooling to room temperature, precipitate was filtered off, washed with cold MeCN and dried.

| | | | MS |
|---|---|---|---|
| Starting compound | Product | Yield (%) | (ESI, m/z) |
| 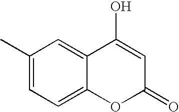 | 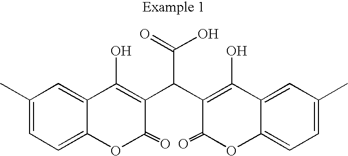 | 89 | 409.2[M + H]⁺ |
| 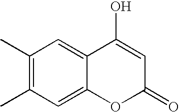 | 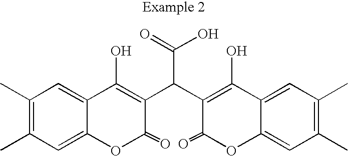 | 83 | 437.0[M + H]⁺ |
| 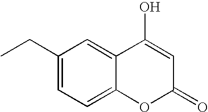 | 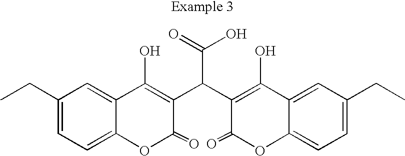 | 79 | 437.3[M + H]⁺ |
| 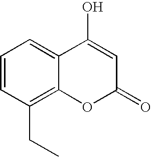 | 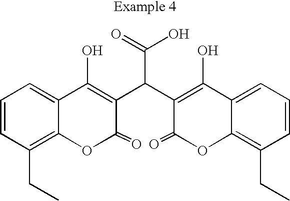 | 48 | 435.1[M − H]⁻ |
| 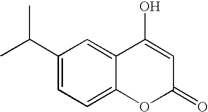 | 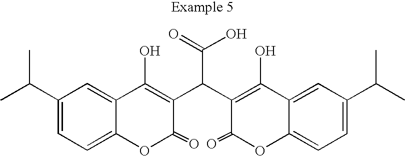 | 55 | 463.1[M − H]⁻ |
| 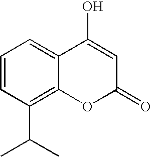 | 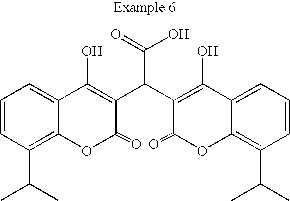 | 71 | 465.3[M + H]⁺ |
| 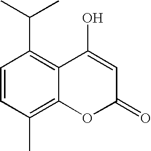 | 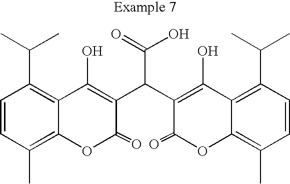 | 75 | 493.1[M + H]⁺ |
| 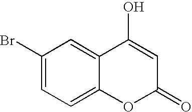 | 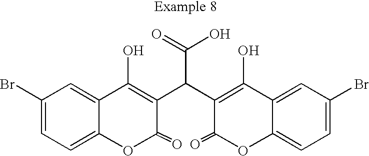 | 75 | 536.7[M + H]⁺ |
| 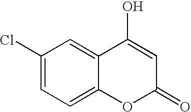 | 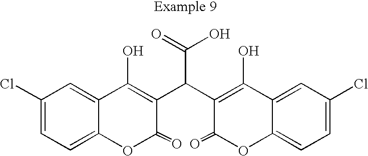 | 87 | 448.8[M + H]⁺ |
| 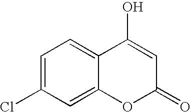 | 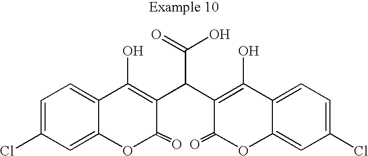 | 48 | 448.9[M + H]⁺ |
| 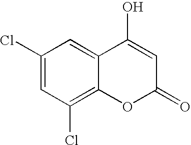 | 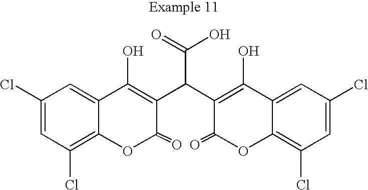 | 72 | 516.8[M + H]⁺ |
| 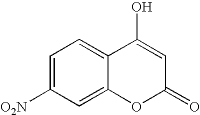 | 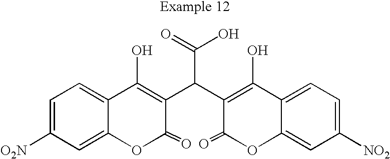 | 26 | 469.0[M − H]⁻ |
| 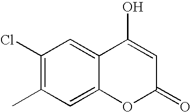 | 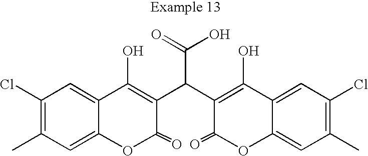 | 97 | 477.1[M + H]⁺ |
| 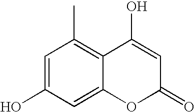 | 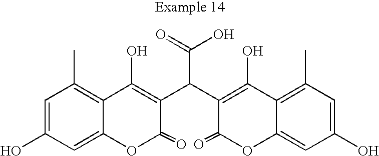 | 51 | 441.0[M + H]⁺ |
| 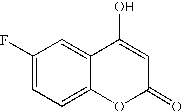 | 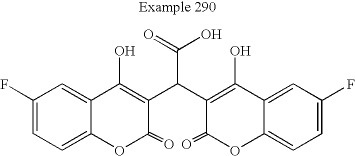 | 67 | 414.9[M − H]⁻ |

### Examples from 15 to 26 and 291

### General Procedure for the Preparation of Compounds of Formula I

wherein A represents a CH—X group, X is an acetyl group and n=0

To a suspension of corresponding 4-hydroxycoumarin (1 equiv.) in EtOH was added 40 wt. % aq. pyruvic aldehyde (4 equiv.). The reaction mixture was heated to reflux, and stirred under reflux until completion of the reaction (2-32 h). Precipitation usually occurred during the reflux. In some cases, after cooling to room temperature, stirring of the reaction mixture was continued until precipitation occurred. Precipitate was filtered off, washed with cold EtOH and dried.

| | | | MS |
|---|---|---|---|
| Starting compound | Product | Yield (%) | (ESI, m/z) |
| 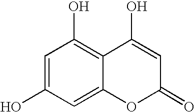 | 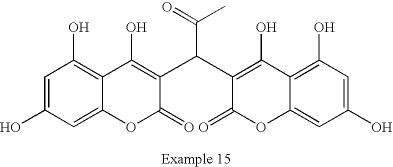 | 55 | 442.8[M + H]⁺ |
| 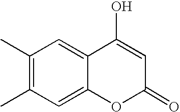 | 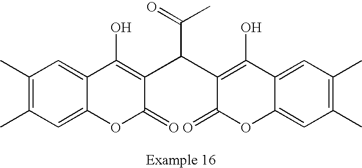 | 76 | 435.2[M + H]⁺ |
| 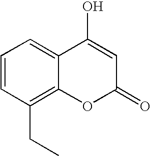 | 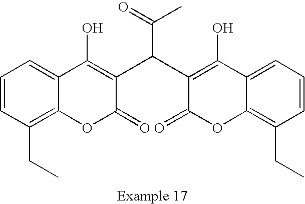 | 35 | 435.0[M + H]⁺ |
| 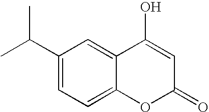 | 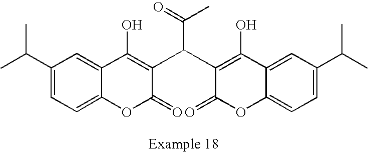 | 19 | 463.1[M + H]⁺ |
| 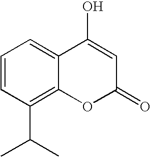 | 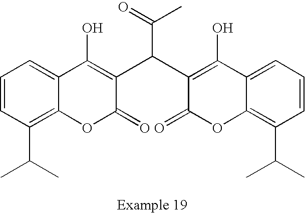 | 39 | 461.2[M − H]⁻ |
| 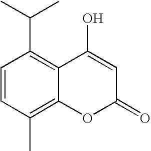 | 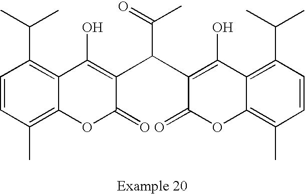 | 16 | 491.3[M + H]⁺ |
| 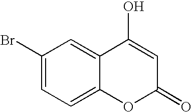 | 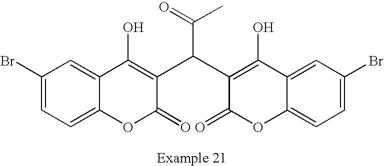 | 14 | 534.9[M + H]⁺ |
| 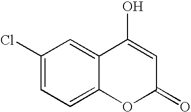 | 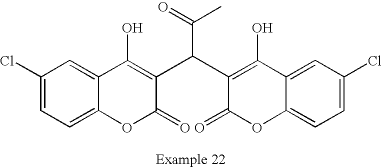 | 53 | 444.9[M − H]⁻ |
| 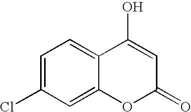 | 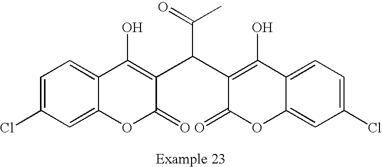 | 13 | 444.9[M − H]⁻ |
| 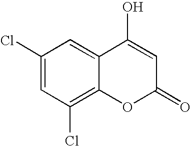 | 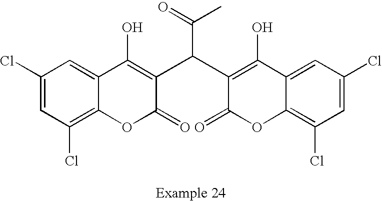 | 5 | 512.8[M − H]⁻ |
| 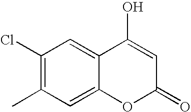 | 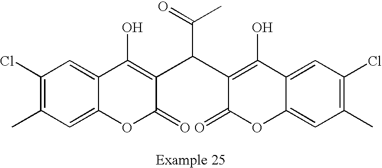 | 81 | 472.9[M − H]⁻ |
| 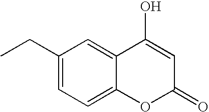 | 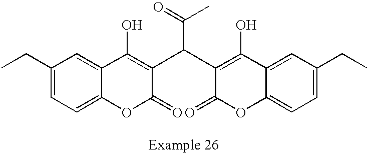 | 37 | 433.0[M − H]⁻ |
| 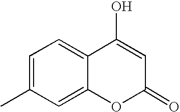 | 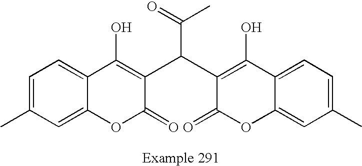 | 79 | 405.0[M − H]⁻ |

### Examples from 27 to 33

### General Procedures for the Preparation of Compounds of Formula I

wherein A represents a CH—X group, X is —CH₂OH and n=0

Method A

To a suspension of corresponding 4-hydroxycoumarin (1 equiv.) in 50 mM aq. tris(hydroxymethyl)aminomethane (Tris) buffer (pH=8.0) was added 1M aqueous solution of NaOH until pH reached 8. To the prepared solution, glycolaldehyde dimer (2 equiv.) was added. The reaction mixture was stirred at room temperature until completion of the reaction (20-60 h). The mixture was acidified with 1M aq. HCl, precipitate was filtered off. Product was purified by trituration or recrystallization from MeOH or EtOH.

Method B

To a suspension of corresponding intramolecular hemiacetal (0.5 mmol) of Formula III, wherein D represents —CHOH and —— denotes a single bond (See, e.g., WO 2005/010006), in tert-BuOH was added cyanoborohydride (1 mmol). The reaction mixture was heated to reflux, and stirred under reflux until completion of the reaction (2 h). Solvent was then evaporated, water and saturated NH₄Cl were added, followed by extraction with AcOEt. Combined organic layers were washed with brine, dried over Na₂SO₄, organic solvent removed under reduced pressure and the residue purified by isocratic elution with CHCl₃:MeOH:AcOH=9:1:0.1 on silica column.

Method C

To a suspension of corresponding intramolecular hemiacetal (0.5 mmol) of Formula III, wherein D represents —CHOH and —— denotes a single bond (See, e.g., WO 2005/010006), in i-PrOH was added cyanoborohydride (1 mmol). The reaction mixture was heated to reflux, and stirred under reflux until completion of the reaction (0.5 h). A mixture of n-hexane:ether=3:1 was added to the reaction mixture, and white precipitate thus formed was filtered off, dissolved in water, followed by addition of 1M HCl. The white precipitate was filtered off, washed with water and additionally purified by trituration with MeOH.

| | | | MS |
|---|---|---|---|
| Starting compound | Product | Yield (%) | (ESI, m/z) |
| 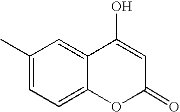 | 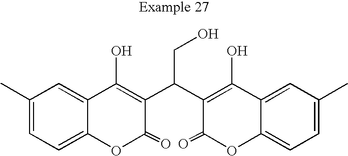 | 70 | 395.2[M + H]⁺ |
| 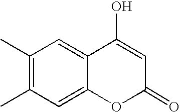 | 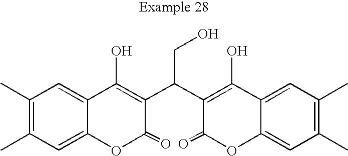 | 46 | 421.1[M − H]⁻ |
| 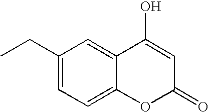 | 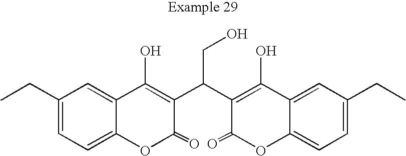 | 60 | 423.3[M + H]⁺ |
| 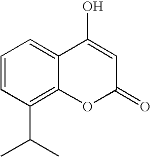 | 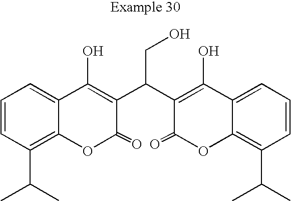 | 44 | 449.1[M − H]⁻ |
| 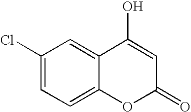 | 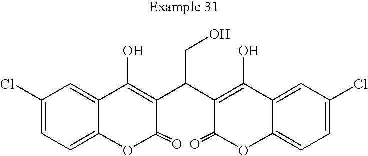 | 41 | 433.2[M − H]⁻ |
| 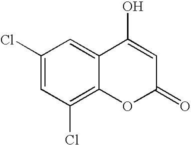 | 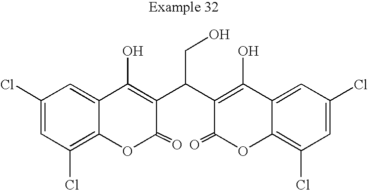 | 81 | 501.1[M − H]⁻ |
| 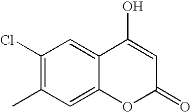 | 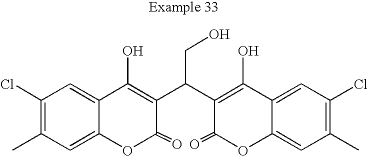 | 42 | 463.1[M + H]⁺ |

### Examples from 34 to 36

### General Procedure for the Preparation of Compounds of Formula I

wherein A represents a CH—X group, X is a

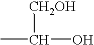

group and n=0

To a suspension of corresponding 4-hydroxycoumarin (1 equiv.) in 50 mM aq. tris(hydroxymethyl)aminomethane (Tris) buffer (pH=8.0) was added 1M aq. NaOH until pH reached 8. To the prepared solution, D,L-glyceraldehyde dimer (2 equiv.) was added. The reaction mixture was stirred at room temperature until completion of the reaction (24-48 h). The mixture was acidified with 1M aq. HCl, precipitate was filtered off. Product was purified by trituration or recrystallization from MeCN or Me₂CO.

| | | | MS |
|---|---|---|---|
| Starting compound | Product | Yield (%) | (ESI, m/z) |
| 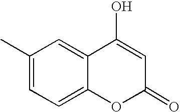 | 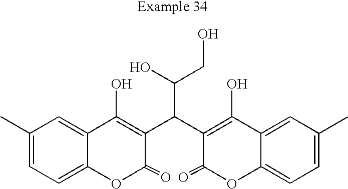 | 21 | 425.2[M + H]⁺ |
| 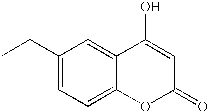 | 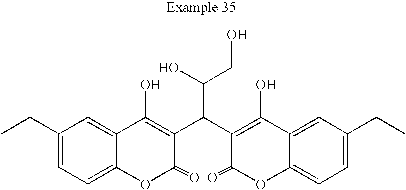 | 12 | 453.3[M + H]⁺ |
| 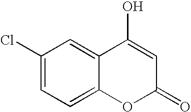 | 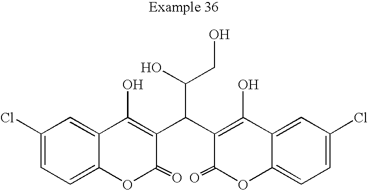 | 21 | 465.0[M + H]⁺ |

### Examples from 37 to 41

### General Procedure for the Preparation of Compounds of Formula I

wherein A represents a carbonyl group and n=1

To a suspension of corresponding 4-hydroxycoumarin (1 equiv.) in water was added aq. solution of D,L-glyceraldehyde dimer (0.5 equiv.). The reaction mixture was heated to reflux, and stirring under reflux was continued until completion of the reaction (5-10 h). After cooling to room temperature, precipitate was filtered off and dried. Product was purified by trituration or recrystallization from MeOH, Me₂CO or CHCl₃.

| | | | MS |
|---|---|---|---|
| Starting compound | Product | Yield (%) | (ESI, m/z) |
| 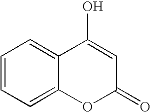 | 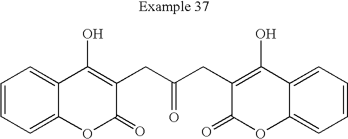 | 21 | 379.2[M + H]⁺ |
| 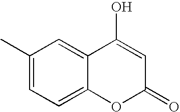 | 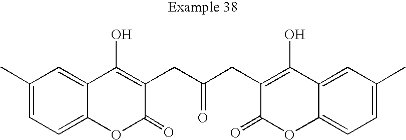 | 5 | 407.2[M + H]⁺ |
| 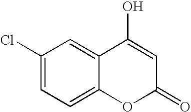 | 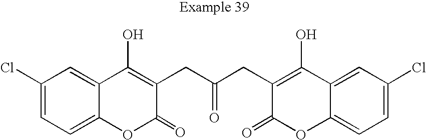 | 5 | 444.9[M − H]⁻ |
| 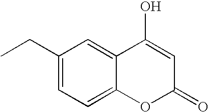 | 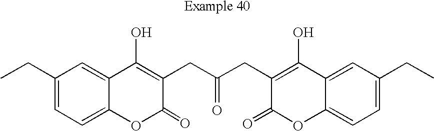 | 7 | 433.0[M − H]⁻ |
| 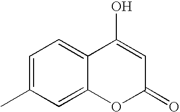 | 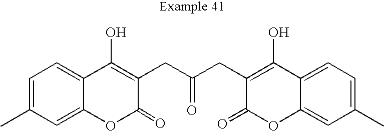 | 7 | 405.0[M − H]⁻ |

### Examples from 42 to 54 and 292 to 294

### General Procedures for the Preparation of Compounds of Formula I

wherein A represents a CH—X group, X is an alkyloxycarbonyl group and n=0

Method A

To a suspension of corresponding carboxylic acid of Formula I, wherein A represents a CH—X group, X is a carboxy group and n=0, in appropriate alcohol (excess), conc. H₂SO₄ was added. The reaction mixture was heated to reflux, and stirring under reflux was continued overnight. After cooling to room temperature, precipitate was filtered off, washed with cold MeOH and dried.

Method B

To a suspension of corresponding lactone of Formula III, wherein D represents a carbonyl group, in 1,4-dioxane, appropriate alcohol (excess) was added. Reaction mixture was heated to 110° C., resulting after a while in a clear solution, and then heating and stirring was continued until the completion of the reaction (several hours). 1,4-Dioxane was removed by evaporation under reduced pressure and then water was added, followed by extraction with AcOEt. Organic layers were washed with brine, dried over Na₂SO₄ and evaporated to smaller volume followed by addition of n-hexane. The precipitate thus formed was filtered off, washed with n-hexane and dried.

| | | Yield | MS |
|---|---|---|---|
| Starting compound | Product | (%) | (ESI, m/z) |
| 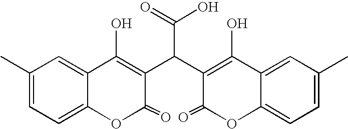 | 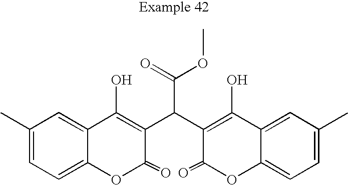 | 83 | 423.2[M + H]⁺ |
| 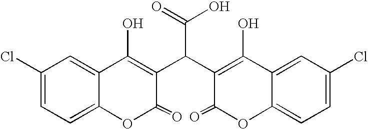 | 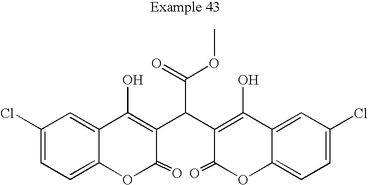 | 91 | 461.4[M − H]⁻ |
| 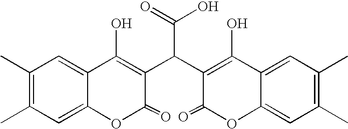 | 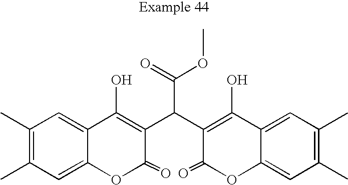 | 98 | 451.3[M + H]⁺ |
| 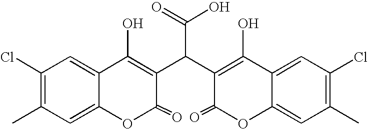 | 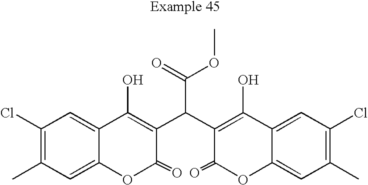 | 90 | 491.2[M + H]⁺ |
| 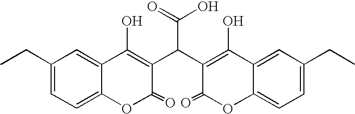 | 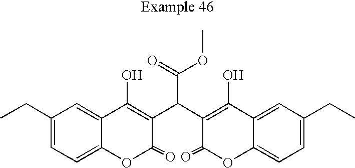 | 68 | 451.3[M + H]⁺ |
| 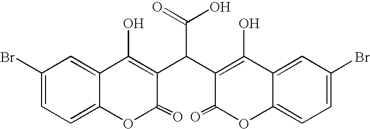 | 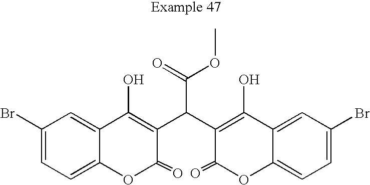 | 90 | 548.7[M − H]⁻ |
| 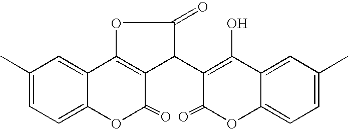 | 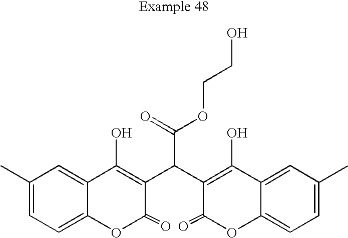 | 77 | 453.3[M + H]⁺ |
| 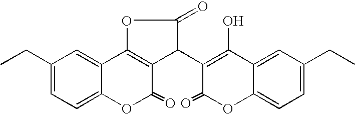 | 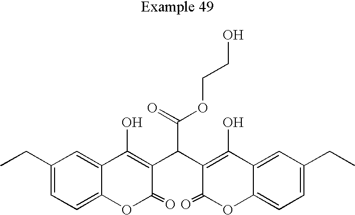 | 81 | 481.3[M + H]⁺ |
| 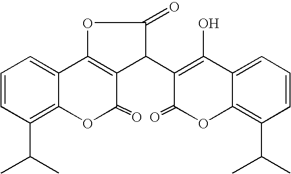 | 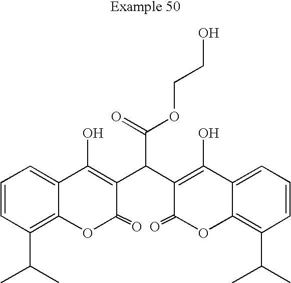 | 72 | 509.4[M + H]⁺ |
| 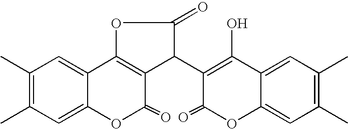 | 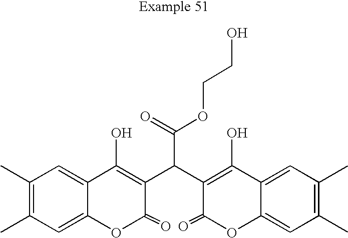 | 80 | 481.3[M + H]⁺ |
| 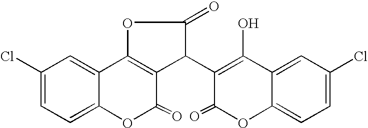 | 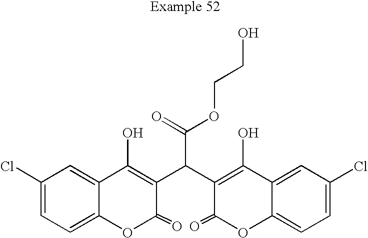 | 80 | 491.2[M − H]⁻ |
| 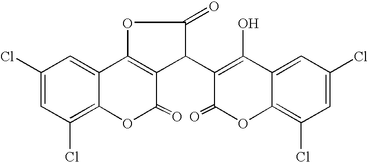 | 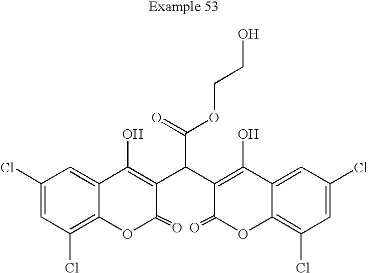 | 75 | 559.3[M − H]⁻ |
| 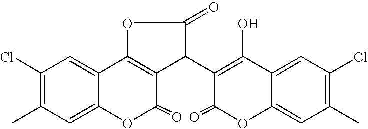 | 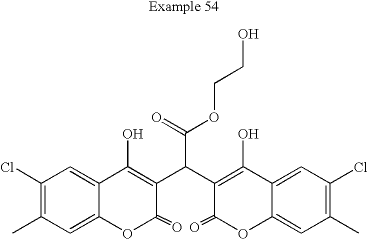 | 70 | 521.2[M + H]⁺ |
| 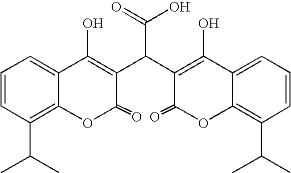 | 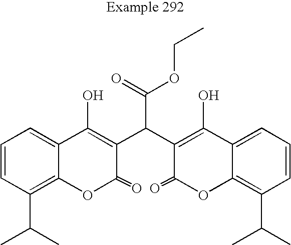 | 74 | 493.0[M + H]⁺ |
| 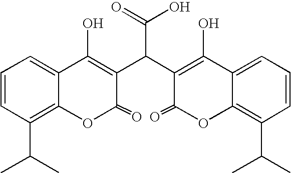 | 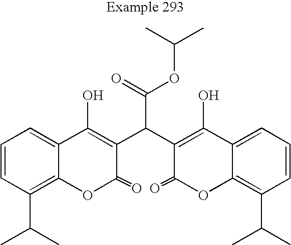 | 49 | 507.2[M + H]⁺ |
| 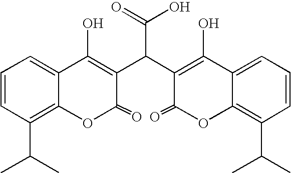 | 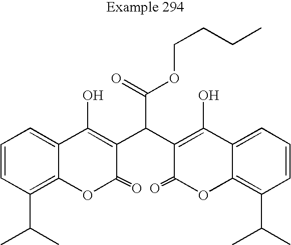 | 52 | 519.2[M − H]⁻ |

### Examples from 55 to 58 and 295

### General Procedure for the Preparation of Compounds of Formula I

wherein A represents a CH—X group, X is an N-alkylcarbamoyl group and n=0

To a suspension of corresponding carboxylic acid (1 equiv.) of Formula I, wherein A represents a CH—X group, X is a carboxy group and n=0, in THF (CH₂Cl₂ may be used instead) was added Et₃N (10 equiv.), resulting in a clear solution. To this solution was added HOBT (1.4 equiv.), followed by appropriate alkyl amine (1.1 equiv.) and EDC (1.5 equiv.). After stirring overnight at room temperature, the precipitate formed was removed by filtration and filtrate was evaporated to dryness. The residue was dissolved in CH₂Cl₂ (AcOEt may be used instead) and washed with saturated NH₄Cl, saturated NaHCO₃ and brine, dried over Na₂SO₄, and concentrated in vacuo. Crude product was purified by chromatography on silica column eluting with CH₂Cl₂/MeOH/NH₄Cl (97/2/0.5→95/4/0.5→91/8/1→90/60/1).

| | | | MS |
|---|---|---|---|
| Starting compound | Product | Yield (%) | (ESI, m/z) |
| 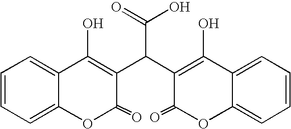 | 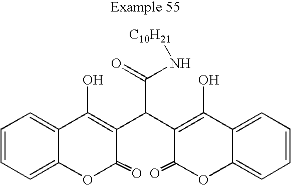 | 47 | 520.1[M + H]⁺ |
| 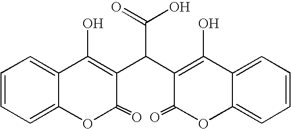 | 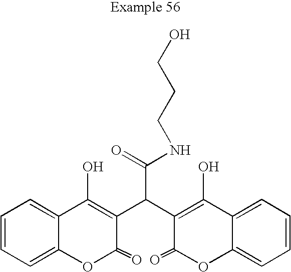 | 11 | 438.2[M + H]⁺ |
| 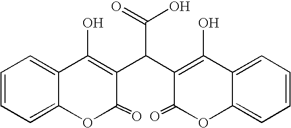 | 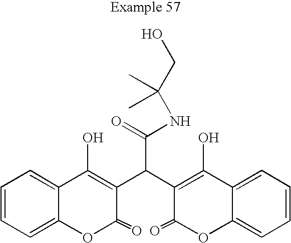 | 60 | 452.0[M + H]⁺ |
| 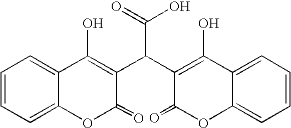 | 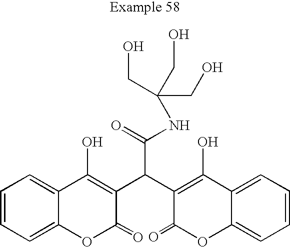 | 54 | 484.2[M + H]⁺ |
| 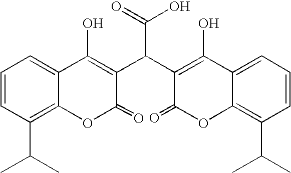 | 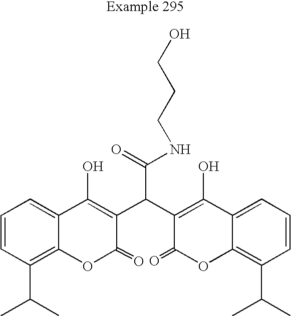 | 64 | 520.0[M − H]⁻ |

### Examples 59 and from 296 to 301

### General Procedure for the Preparation of Compounds of Formula I

wherein A represents a CH—X group, X is a —C(—N—R₅)R₆ group and n=0

To a suspension of corresponding intramolecular hemiacetal (1 equiv.) of Formula III, wherein D represents a CHOH group and —— denotes a single bond (See, e.g., WO 2005/010006), in i-PrOH (optionally EtOH, MeCN or water) hydroxylamine (2 equiv.) was added (or O-alkylhydroxylamine, e.g. O-methylhydroxylamine, hydrazine, alkylhydrazine, e.g. methylhydrazine, aryl amine, e.g. aniline, or arylhydrazine, e.g. phenylhydrazine) resulting in a clear solution. Stirring at room temperature (optionally at elevated temperature if necessary) was continued until the completion of the reaction (1 h to several hours), solvent was evaporated and to the residue AcOEt was added, organic layer was washed with saturated aq. NH₄Cl, saturated aq. NaHCO₃, brine and dried over anhydrous Na₂SO₄. Evaporation of the solvent to the smaller volume resulted in precipitation of the product, which was filtered of and dried.

| | | Yield | MS |
|---|---|---|---|
| Starting compound | Product | (%) | (ESI, m/z) |
| 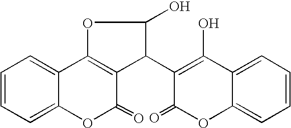 | 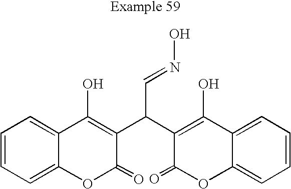 | 45 | 380.0[M + H]⁺ |
| 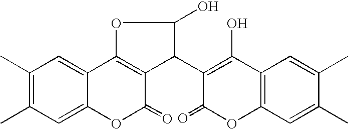 | 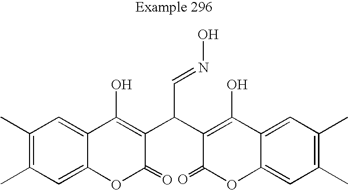 | 59 | 434.0[M − H]⁻ |
| 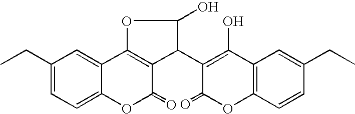 | 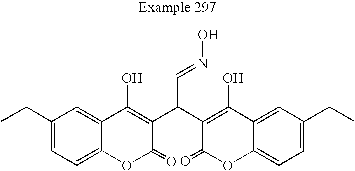 | 95 | 434.0[M − H]⁻ |
| 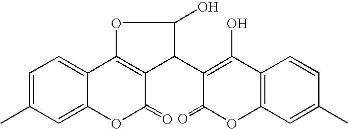 | 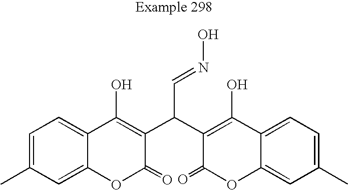 | 98 | 405.9[M − H]⁻ |
| 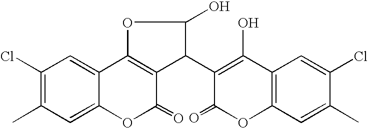 | 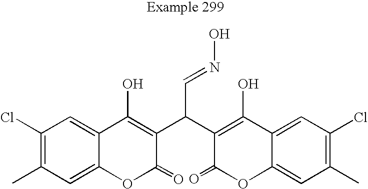 | 95 | 473.9[M − H]⁻ |
| 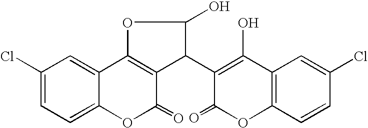 | 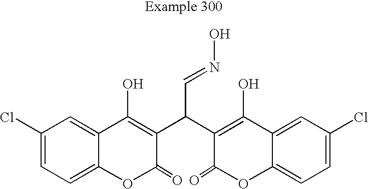 | 88 | 445.8[M − H]⁻ |
| 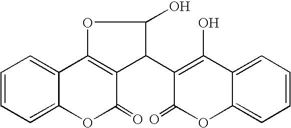 | 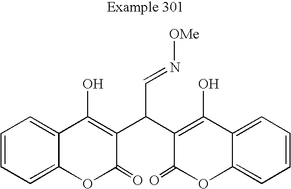 | 71 | 393.9[M + H]⁺ |

### Examples from 60 to 246 and from 302 to 359

### General Procedure for the Preparation of Compounds of Formula I

wherein A represents a CH—X group, X is aryl or heteroaryl and n=0

To a suspension of corresponding 4-hydroxycoumarin (1 equiv.) in EtOH was added solution of appropriate aldehyde (1.22 equiv.) in EtOH. The reaction mixture was heated to 55° C., and stirring at 55° C. was continued until completion of the reaction (12-96 h). Precipitation usually occurred during the heating. In some cases, after cooling to room temperature, stirring of the reaction mixture was continued until precipitation occurred. Precipitate was filtered off, washed with cold EtOH and dried.

| | | | Yield | MS |
|---|---|---|---|---|
| Coumarin | Aldehyde | Product | (%) | (ESI, m/z) |
| 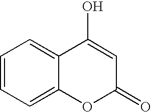 | 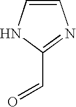 | 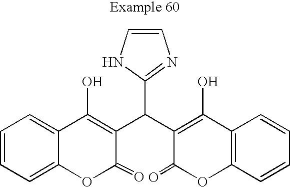 | 50.2 | 403.2[M + H]⁺ |
| 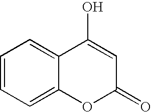 | 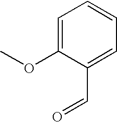 | 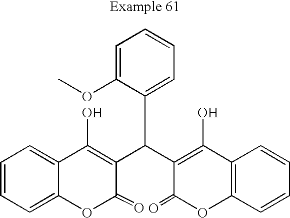 | 11.6 | 443.1[M + H]⁺ |
| 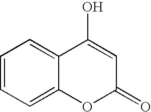 | 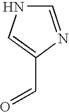 | 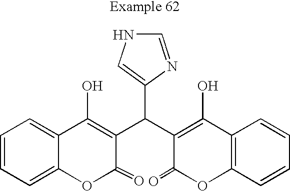 | 38.7 | 464.3[M + H]⁺ |
| 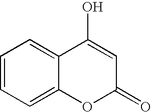 | 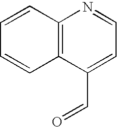 | 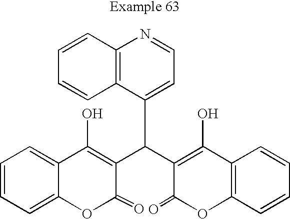 | 19.2 | 403.1[M + H]⁺ |
| 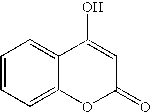 | 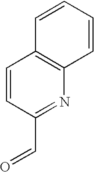 | 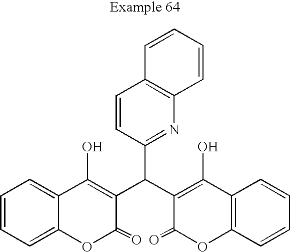 | 39.5 | 464.3[M + H]⁺ |
| 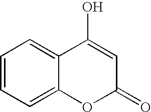 | 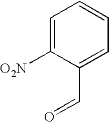 | 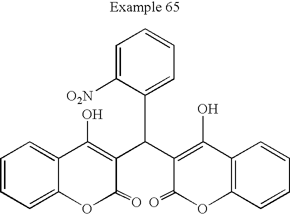 | 40.9 | 464.2[M + H]⁺ |
| 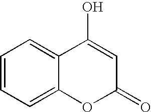 | 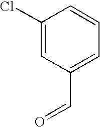 | 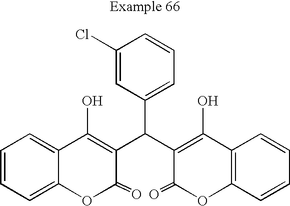 | 11.1 | 458.2[M + H]⁺ |
| 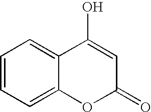 | 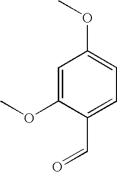 | 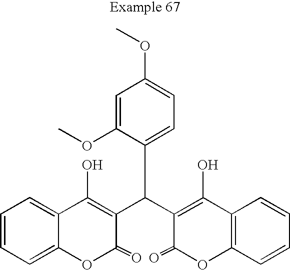 | 14.3 | 457.2[M + H]⁺ |
| 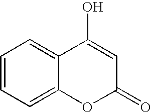 | 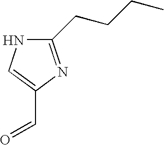 | 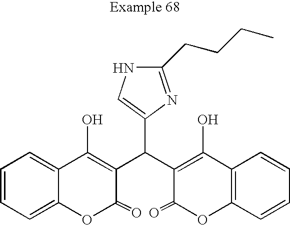 | 28.8 | 473.1[M + H]⁺ |
| 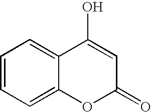 | 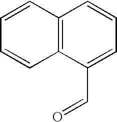 | 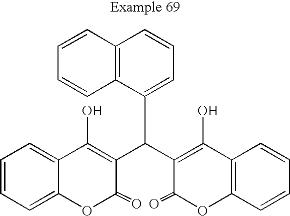 | 34.1 | 459.3[M + H]⁺ |
| 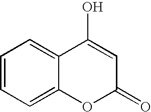 | 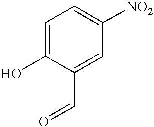 | 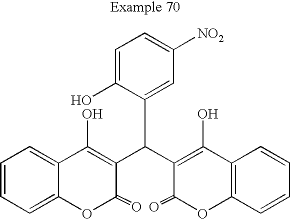 | 6.3 | 463.2[M + H]⁺ |
| 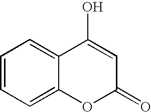 | 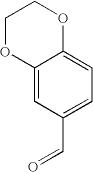 | 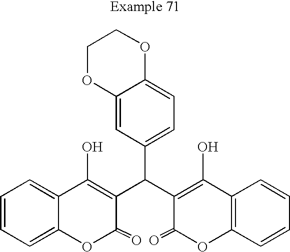 | 99.1 | 471.3[M + H]⁺ |
| 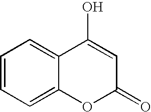 | 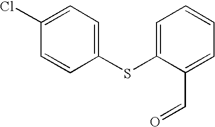 | 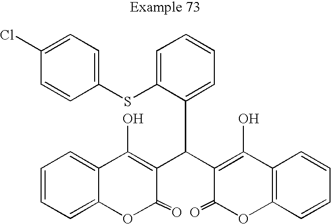 | 16.8 | 555.2[M + H]⁺ |
| 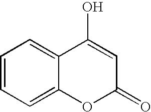 | 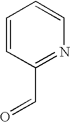 | 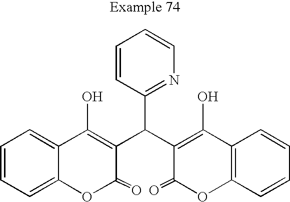 | 69.6 | 414.0[M + H]⁺ |
| 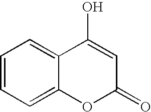 | 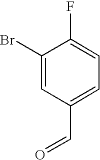 | 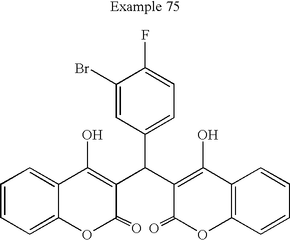 | 42.2 | 509.1[M + H]⁺ |
| 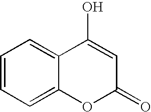 | 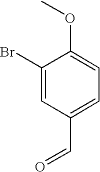 | 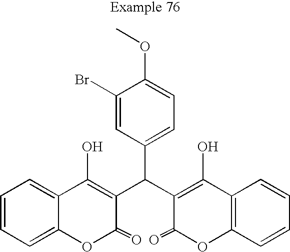 | 83.6 | 521.2[M + H]⁺ |
| 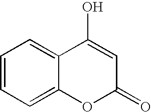 | 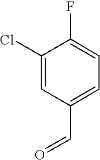 | 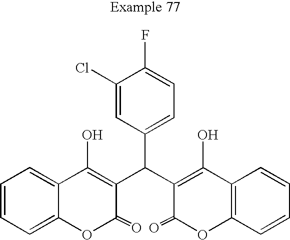 | 43.3 | 465.1[M + H]⁺ |
| 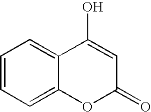 | 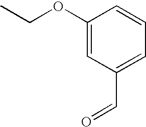 | 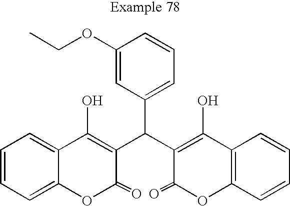 | 53.5 | 457.3[M + H]⁺ |
| 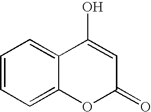 | 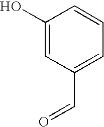 | 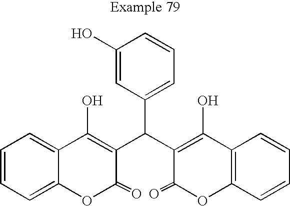 | 90.7 | 429.2[M + H]⁺ |
| 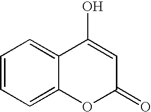 | 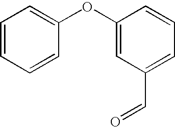 | 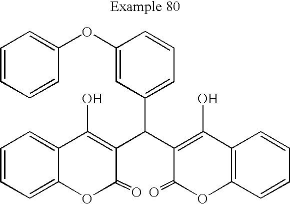 | 60.0 | 505.4[M + H]⁺ |
| 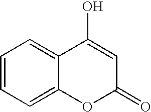 | 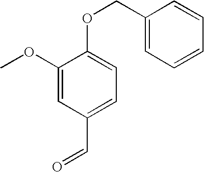 | 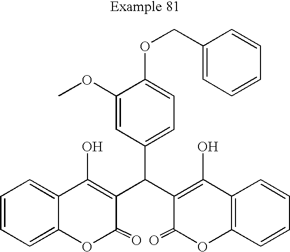 | 78.1 | 549.3[M + H]⁺ |
| 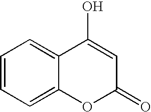 | 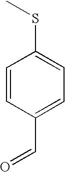 | 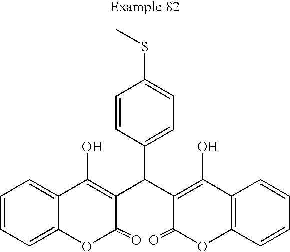 | 76.2 | 459.2[M + H]⁺ |
| 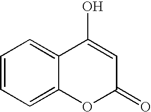 | 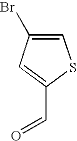 | 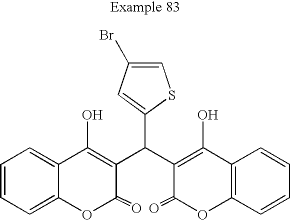 | 68.9 | 497.0[M + H]⁺ |
| 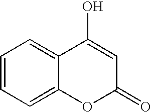 | 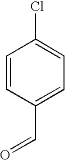 | 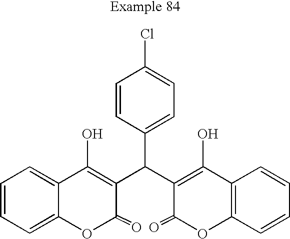 | 51.5 | 447.2[M + H]⁺ |
| 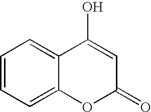 | 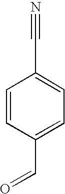 | 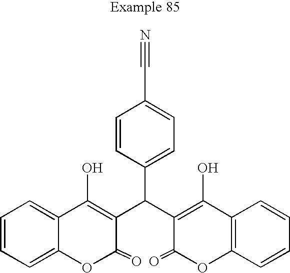 | 39.8 | 438.2[M + H]⁺ |
| 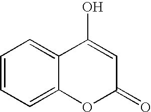 | 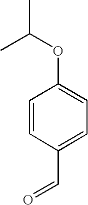 | 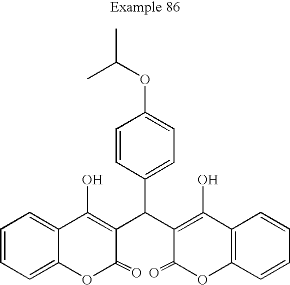 | 85.9 | 471.3[M + H]⁺ |
| 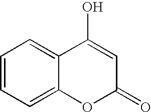 | 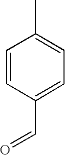 | 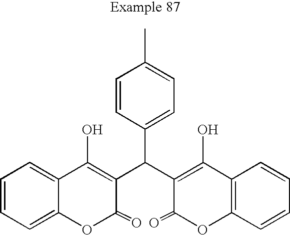 | 72.0 | 427.3[M + H]⁺ |
| 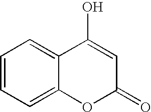 | 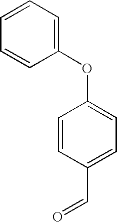 | 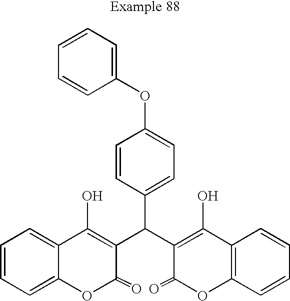 | 93.2 | 505.3[M + H]⁺ |
| 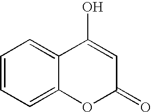 | 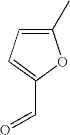 | 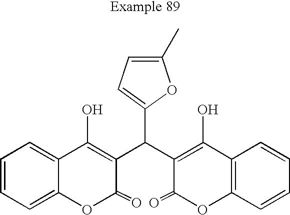 | 34.3 | 417.3[M + H]⁺ |
| 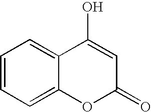 | 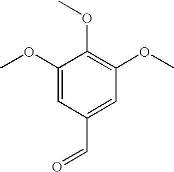 | 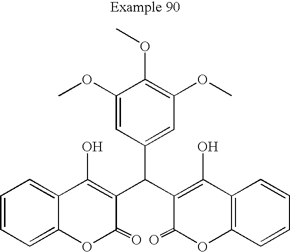 | 9.6 | 525.2[M + Na]⁺ |
| 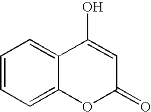 | 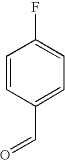 | 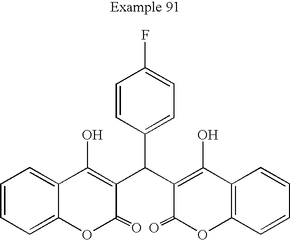 | 27.6 | 453.2[M + Na]⁺ |
| 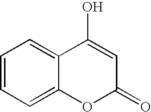 | 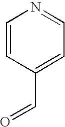 | 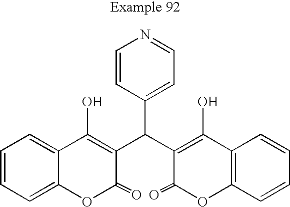 | 29.5 | 436.2[M + Na]⁺ |
| 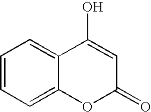 | 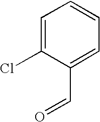 | 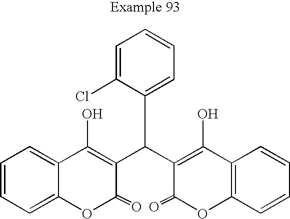 | 25.9 | 447.2[M + H]⁺ |
| 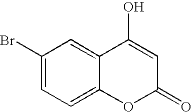 | 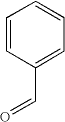 | 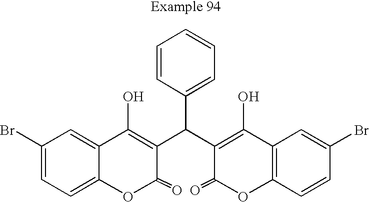 | 40.4 | 567.0[M + H]⁺ |
| 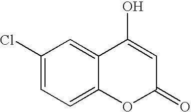 | 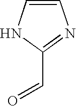 | 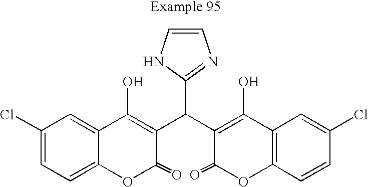 | 94.9 | 471.1[M + H]⁺ |
| 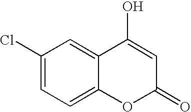 | 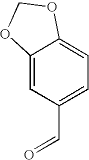 | 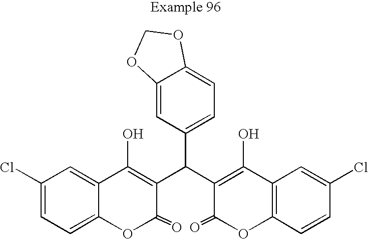 | 83.4 | 523.1[M − H]⁻ |
| 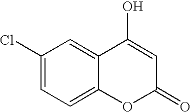 | 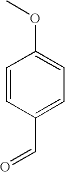 | 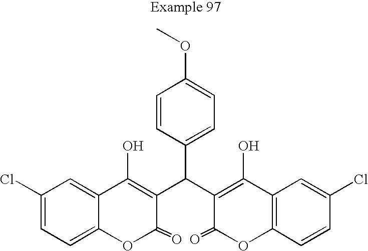 | 79.5 | 509.2[M − H]⁻ |
| 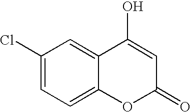 | 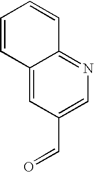 | 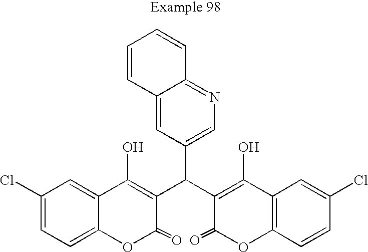 | 61.6 | 532.2[M + H]⁺ |
| 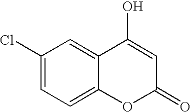 | 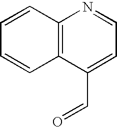 | 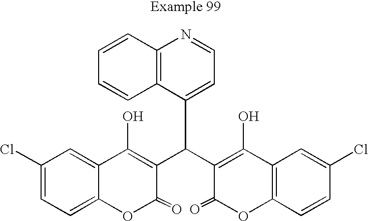 | 64.6 | 532.1[M + H]⁺ |
| 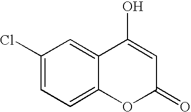 | 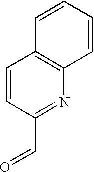 | 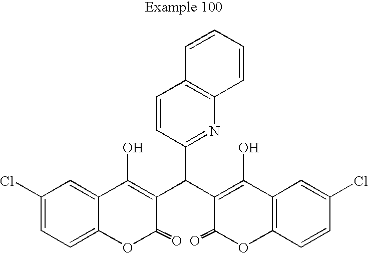 | 75.9 | 532.2[M + H]⁺ |
| 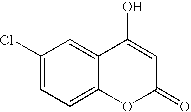 | 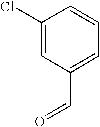 | 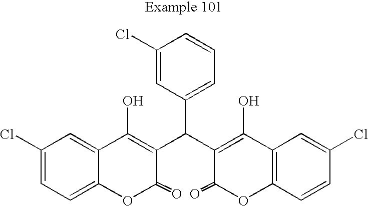 | 21.3 | 513.2[M − H]⁻ |
| 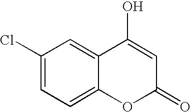 | 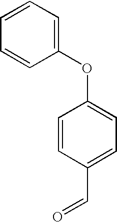 | 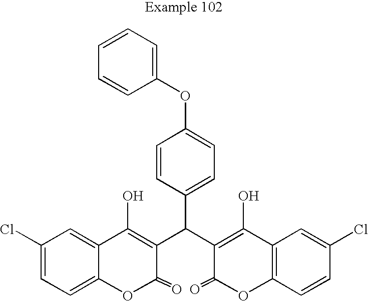 | 77.7 | 571.2[M − H]⁻ |
| 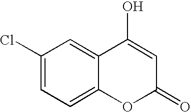 | 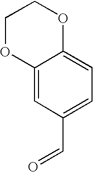 | 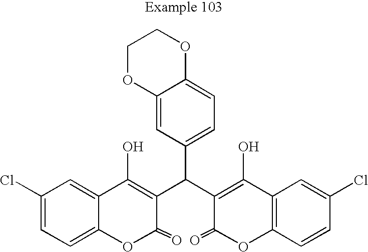 | 86.5 | 537.3[M − H]⁻ |
| 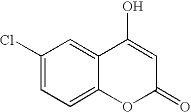 | 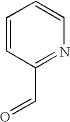 | 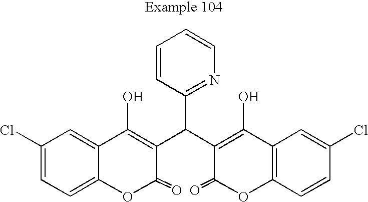 | 83.5 | 482.2[M + H]⁺ |
| 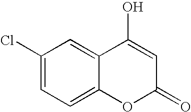 | 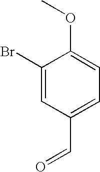 | 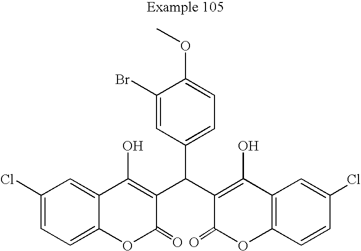 | 78.8 | 587.2[M − H]⁻ |
| 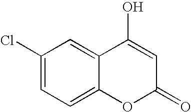 | 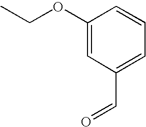 | 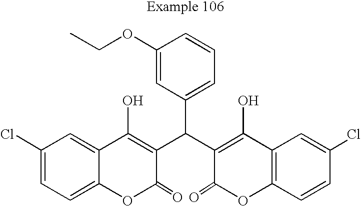 | 83.6 | 523.3[M − H]⁻ |
| 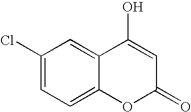 | 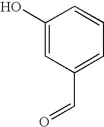 | 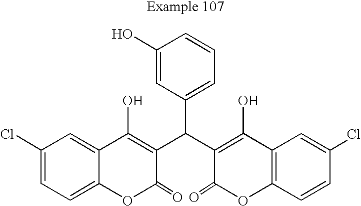 | 14.7 | 495.3[M − H]⁻ |
| 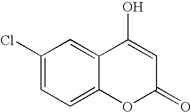 | 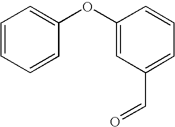 | 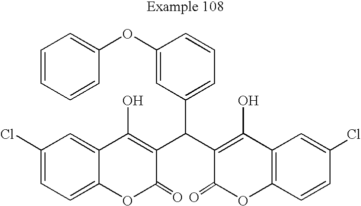 | 56.5 | 571.2[M − H]⁻ |
| 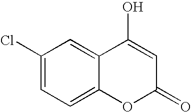 | 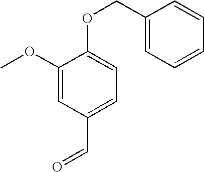 | 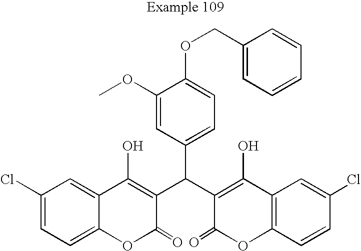 | 86.2 | 615.3[M − H]⁻ |
| 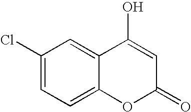 | 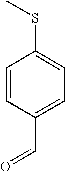 | 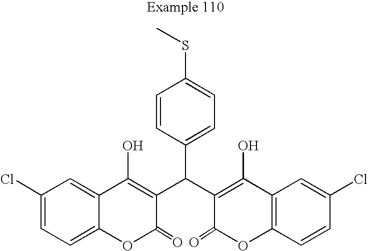 | 77.8 | 525.2[M − H]⁻ |
| 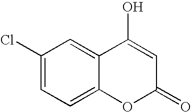 | 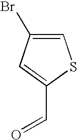 | 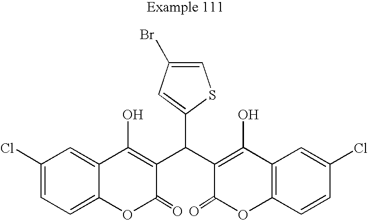 | 36.7 | 563.0[M − H]⁻ |
| 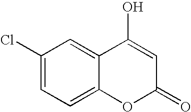 | 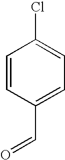 | 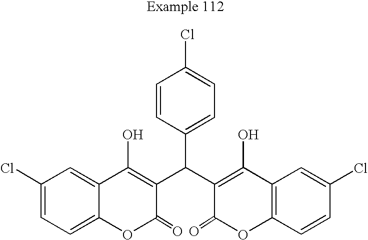 | 34.0 | 513.2[M − H]⁻ |
| 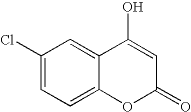 | 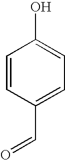 | 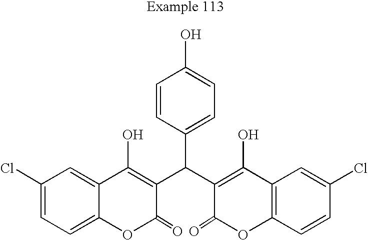 | 86.5 | 494.9[M − H]⁻ |
| 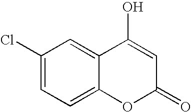 | 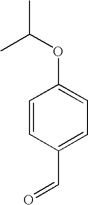 | 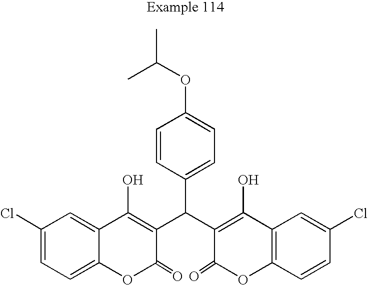 | 80.0 | 537.3[M − H]⁻ |
| 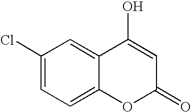 | 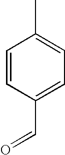 | 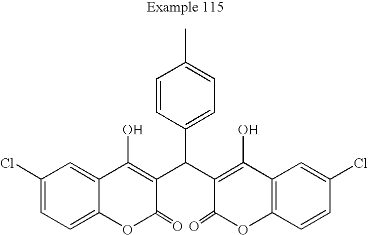 | 74.7 | 493.2[M − H]⁻ |
| 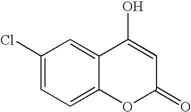 | 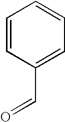 | 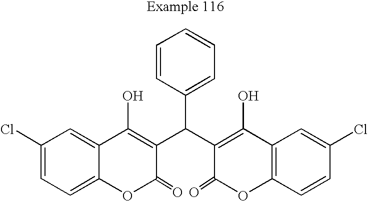 | 52.2 | 479.2[M + H]⁺ |
| 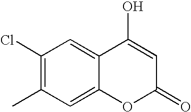 | 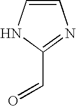 | 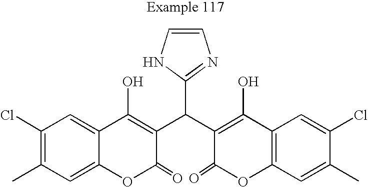 | 89.1 | 498.6[M + H]⁺ |
| 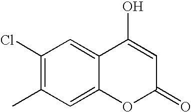 | 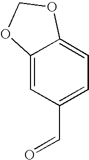 | 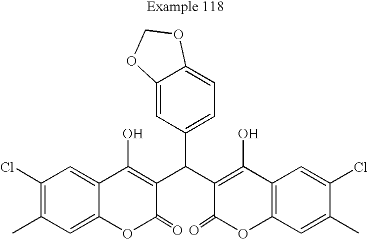 | 81.7 | 550.9[M − H]⁻ |
| 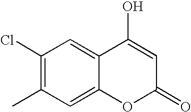 | 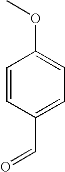 | 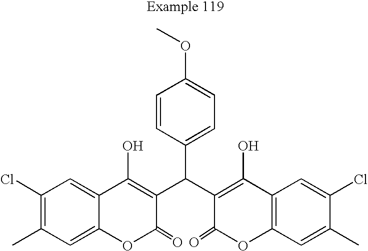 | 79.9 | 536.9[M − H]⁻ |
| 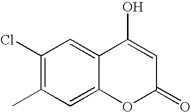 | 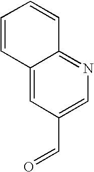 | 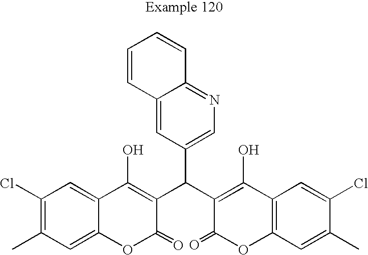 | 76.2 | 559.6[M + H]⁺ |
| 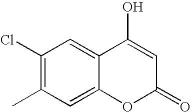 | 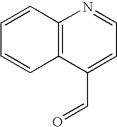 | 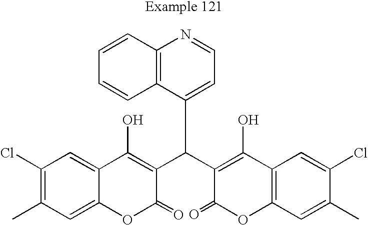 | 85.9 | 559.6[M + H]⁺ |
| 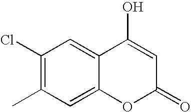 | 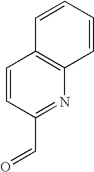 | 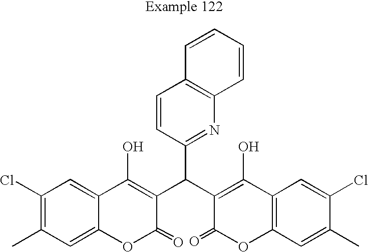 | 79.7 | 559.7[M + H]⁺ |
| 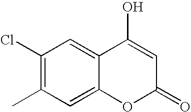 | 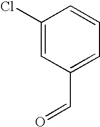 | 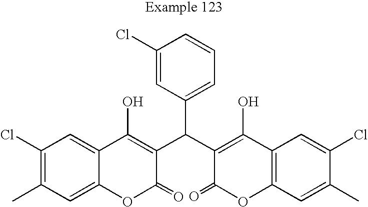 | 14.5 | 540.7[M − H]⁻ |
| 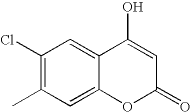 | 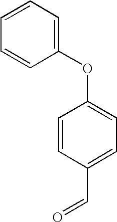 | 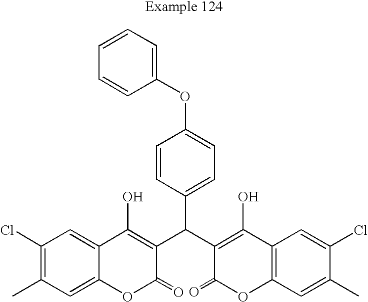 | 88.5 | 598.9[M − H]⁻ |
| 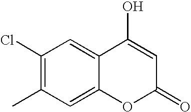 | 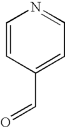 | 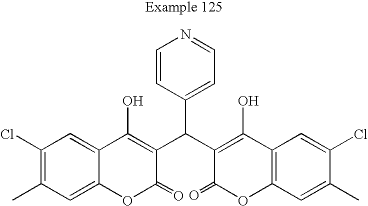 | 86.7 | 509.9[M + H]⁺ |
| 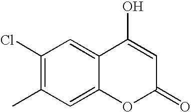 | 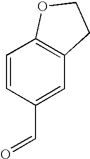 | 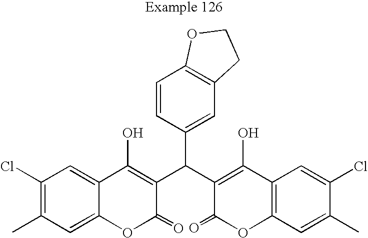 | 84.0 | 548.9[M − H]⁻ |
| 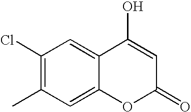 | 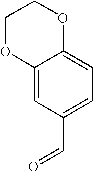 | 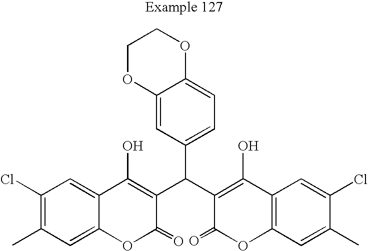 | 84.9 | 564.9[M − H]⁻ |
| 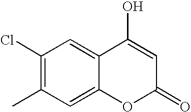 | 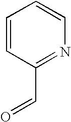 | 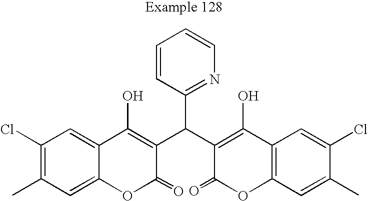 | 88.3 | 509.7[M + H]⁺ |
| 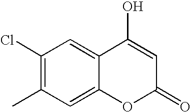 | 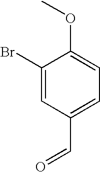 | 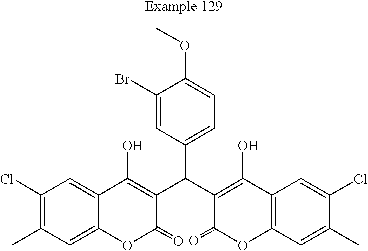 | 52.2 | 614.7[M − H]⁻ |
| 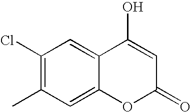 | 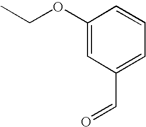 | 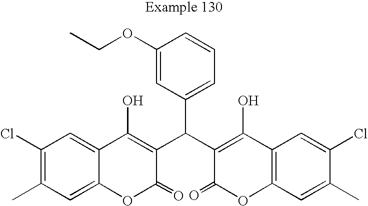 | 77.9 | 550.9[M − H]⁻ |
| 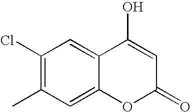 | 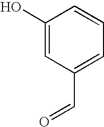 | 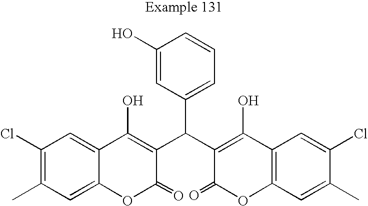 | 75.9 | 522.8[M − H]⁻ |
| 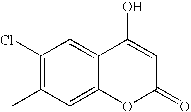 | 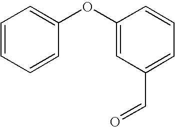 | 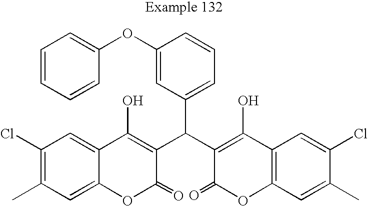 | 50.4 | 599.04[M − H]⁻ |
| 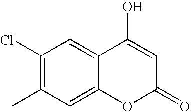 | 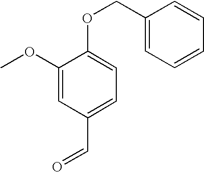 | 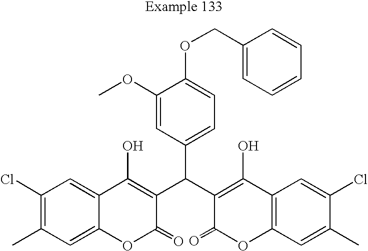 | 60.0 | 642.9[M − H]⁻ |
| 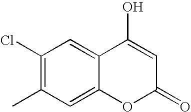 | 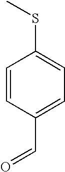 | 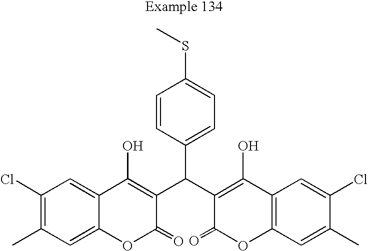 | 82.7 | 552.9[M − H]⁻ |
| 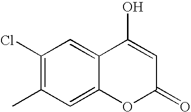 | 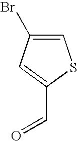 | 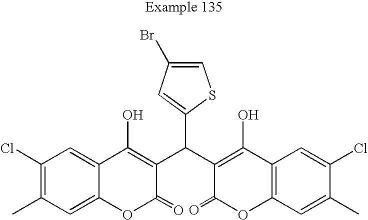 | 19.9 | 590.7[M − H]⁻ |
| 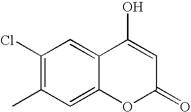 | 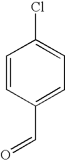 | 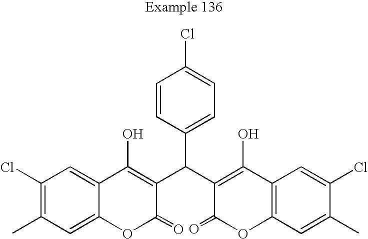 | 42.7 | 540.8[M − H]⁻ |
| 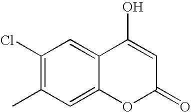 | 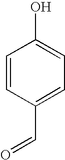 | 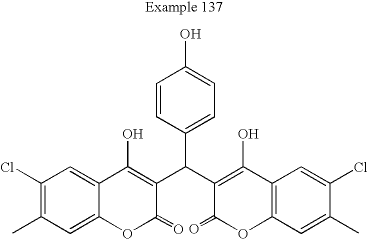 | 72.9 | 522.9[M − H]⁻ |
| 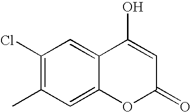 | 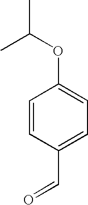 | 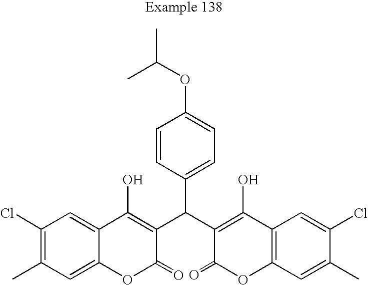 | 73.7 | 565.0[M − H]⁻ |
| 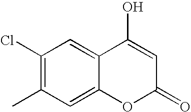 | 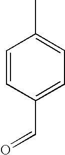 | 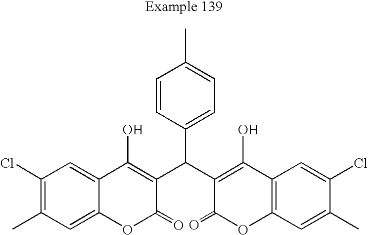 | 69.7 | 520.9[M − H]⁻ |
| 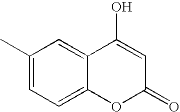 | 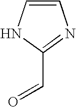 | 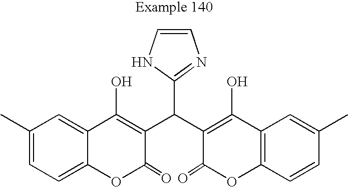 | 36.4 | 431.2[M + H]⁺ |
| 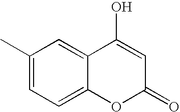 | 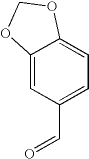 | 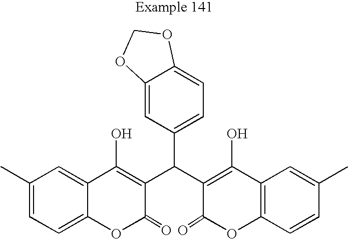 | 88.8 | 485.2[M + H]⁺ |
| 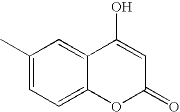 | 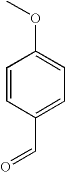 | 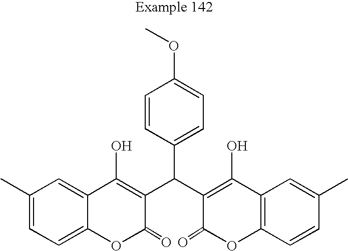 | 88.3 | 471.2[M + H]⁺ |
| 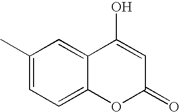 | 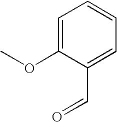 | 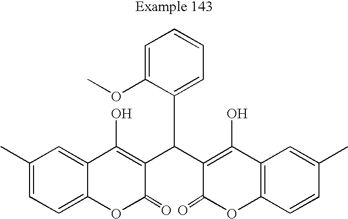 | 93.5 | 471.2[M + H]⁺ |
| 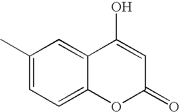 | 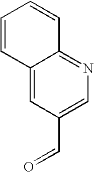 | 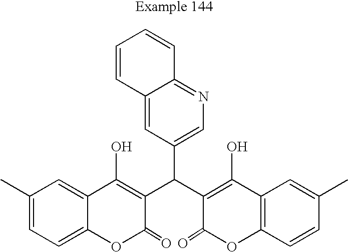 | 68.0 | 492.3[M + H]⁺ |
| 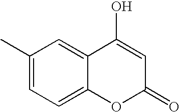 | 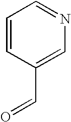 | 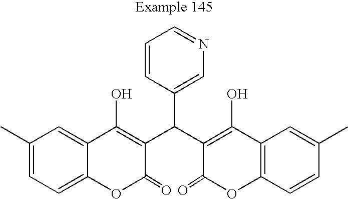 | 82.1 | 441.9[M + H]⁺ |
| 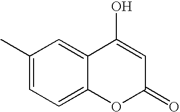 | 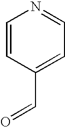 | 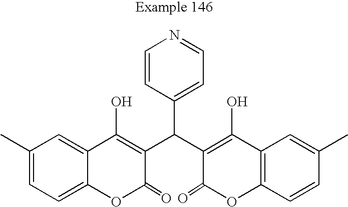 | 64.2 | 442.2[M + H]⁺ |
| 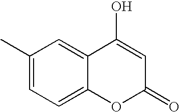 | 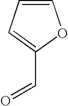 | 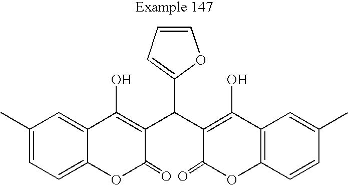 | 25.4 | 431.2[M + H]⁺ |
| 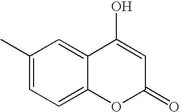 | 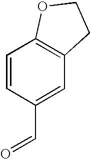 | 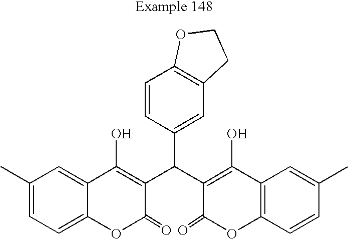 | 72.4 | 483.2[M + H]⁺ |
| 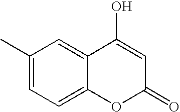 | 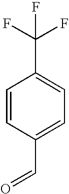 | 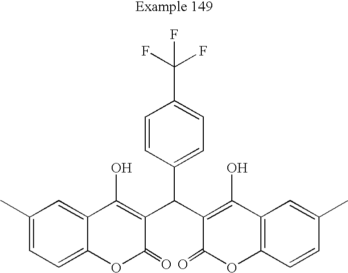 | 30.5 | 507.3[M − H]⁻ |
| 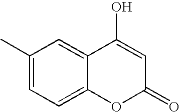 | 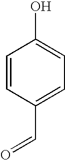 | 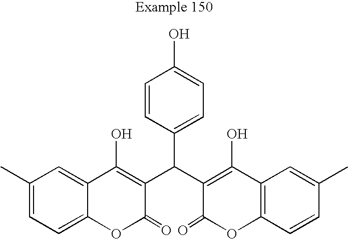 | 74.2 | 457.2[M + H]⁺ |
| 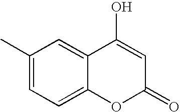 | 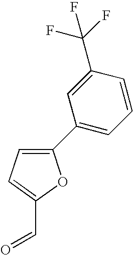 | 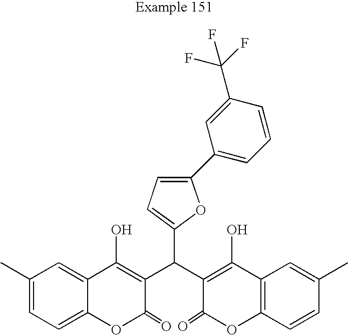 | 87.6 | 575.1[M + H]⁺ |
| 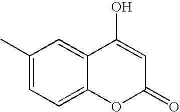 | 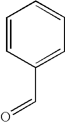 | 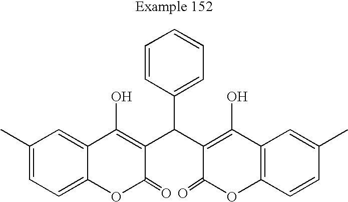 | 16.6 | 441.1[M + H]⁺ |
| 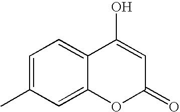 | 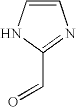 | 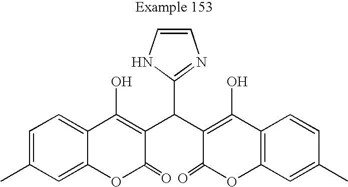 | 76.3 | 430.9[M + H]⁺ |
| 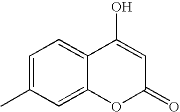 | 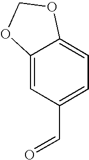 | 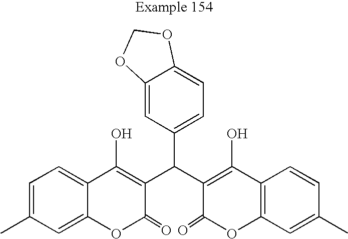 | 86.2 | 485.0[M + H]⁺ |
| 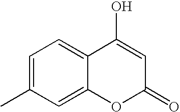 | 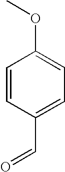 | 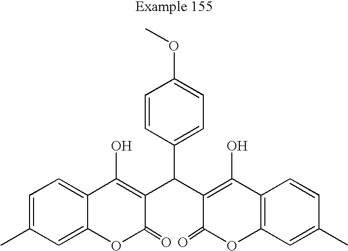 | 89.3 | 471.0[M + H]⁺ |
| 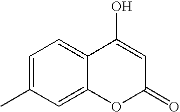 | 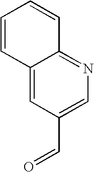 | 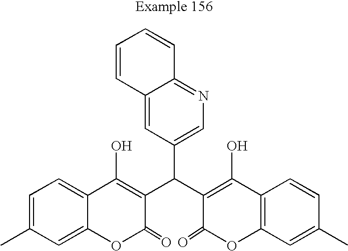 | 70.1 | 492.0[M + H]⁺ |
| 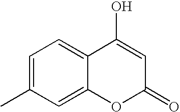 | 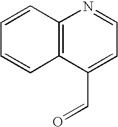 | 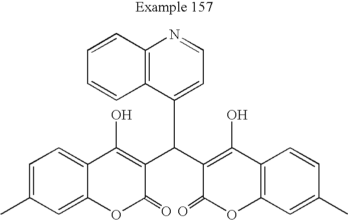 | 70.1 | 492.0[M + H]⁺ |
| 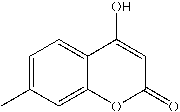 | 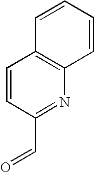 | 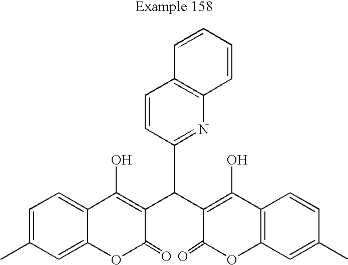 | 73.2 | 492.0[M + H]⁺ |
| 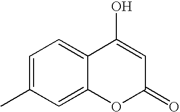 | 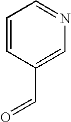 | 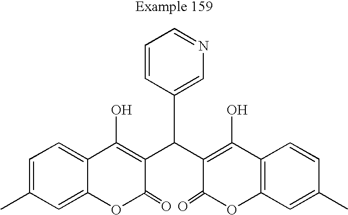 | 78.9 | 442.0[M + H]⁺ |
| 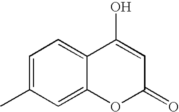 | 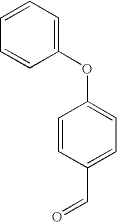 | 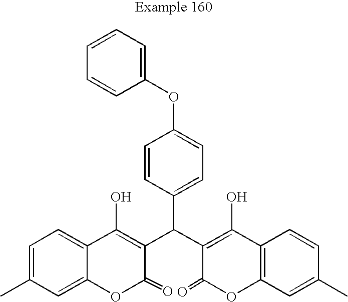 | 85.7 | 533.0[M + H]⁺ |
| 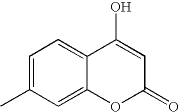 | 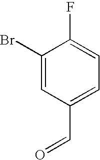 | 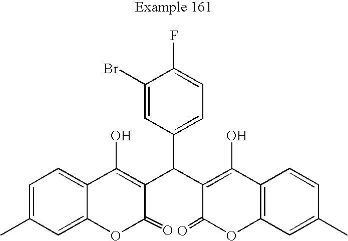 | 43.4 | 536.9[M + H]⁺ |
| 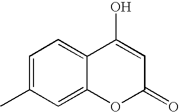 | 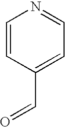 | 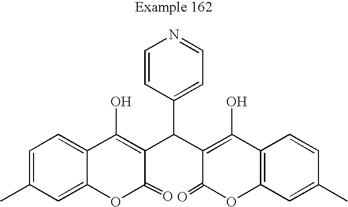 | 76.5 | 442.0[M + H]⁺ |
| 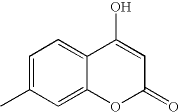 | 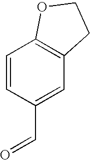 | 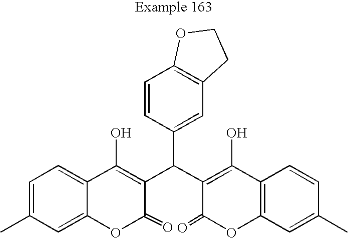 | 80.2 | 483.0[M + H]⁺ |
| 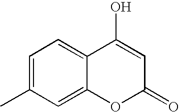 | 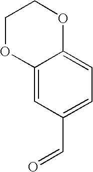 | 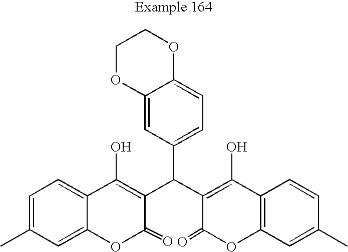 | 80.0 | 499.0[M + H]⁺ |
| 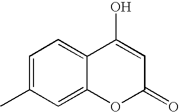 | 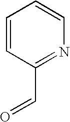 | 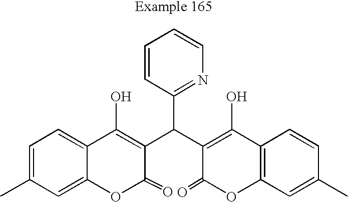 | 89.3 | 442.0[M + H]⁺ |
| 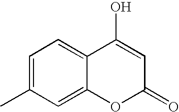 | 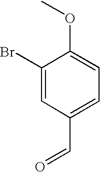 | 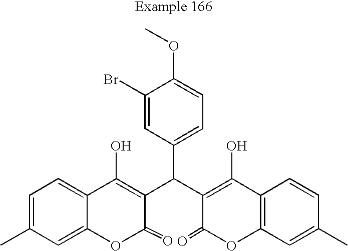 | 82.9 | 548.9[M + H]⁺ |
| 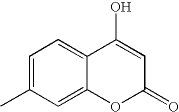 | 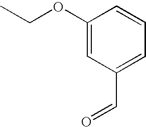 | 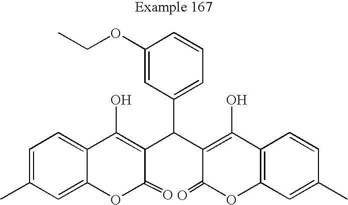 | 21.4 | 485.0[M + H]⁺ |
| 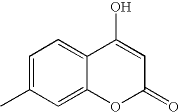 | 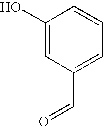 | 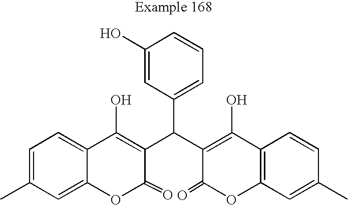 | 50.5 | 457.0[M + H]⁺ |
| 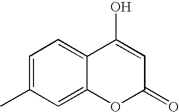 | 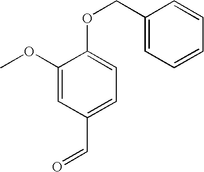 | 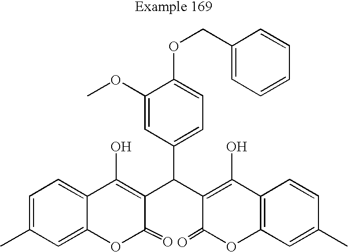 | 82.4 | 577.0[M + H]⁺ |
| 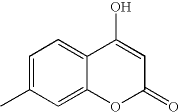 | 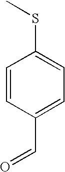 | 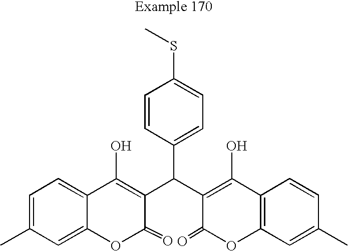 | 86.6 | 486.9[M + H]⁺ |
| 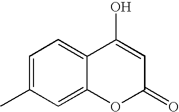 | 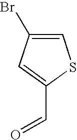 | 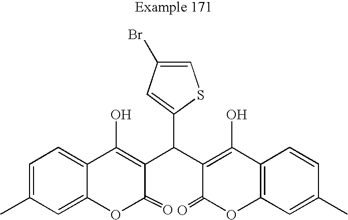 | 34.5 | 524.8[M + H]⁺ |
| 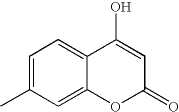 | 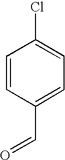 | 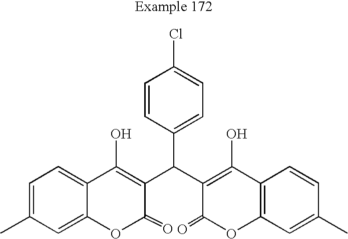 | 78.3 | 474.9[M + H]⁺ |
| 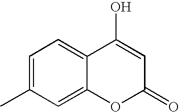 | 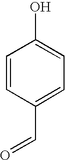 | 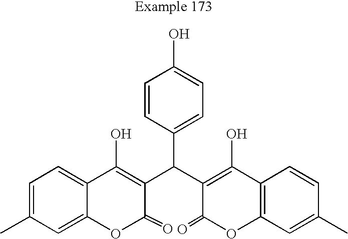 | 60.3 | 457.0[M + H]⁺ |
| 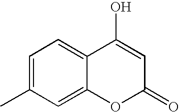 | 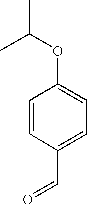 | 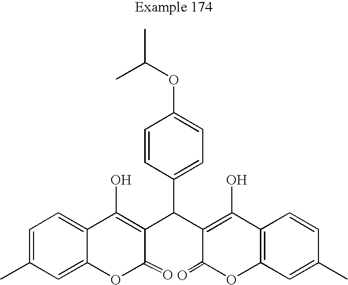 | 34.2 | 499.0[M + H]⁺ |
| 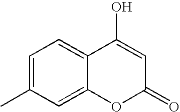 | 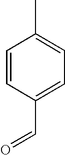 | 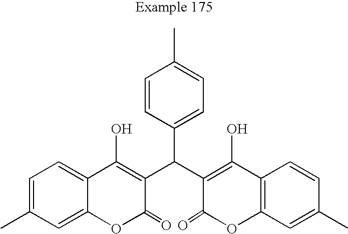 | 87.2 | 455.0[M + H]⁺ |
| 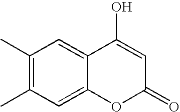 | 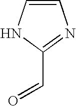 | 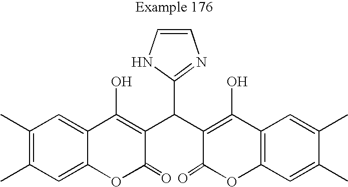 | 73.9 | 459.3[M + H]⁺ |
| 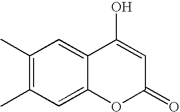 | 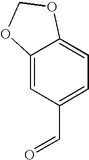 | 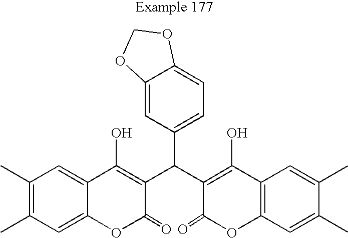 | 81.5 | 513.3[M + H]⁺ |
| 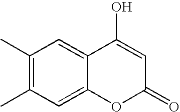 | 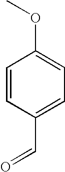 | 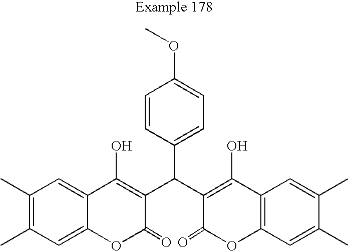 | 81.5 | 499.3[M + H]⁺ |
| 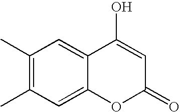 | 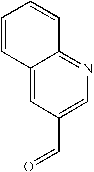 | 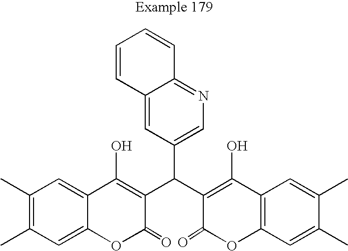 | 64.3 | 520.4[M + H]⁺ |
| 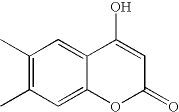 | 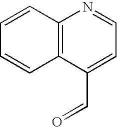 | 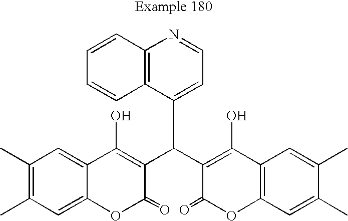 | 78.9 | 520.4[M + H]⁺ |
| 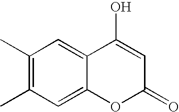 | 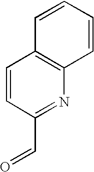 | 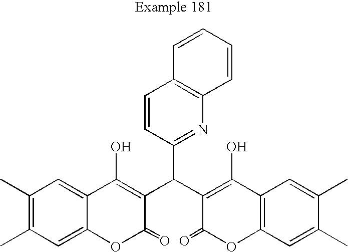 | 64.1 | 520.4[M + H]⁺ |
| 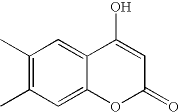 | 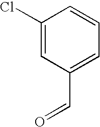 | 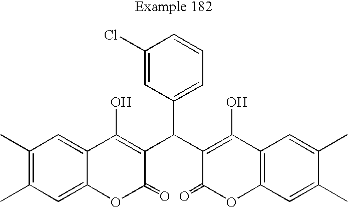 | 16.4 | 503.1[M + H]⁺ |
| 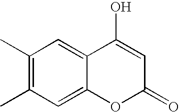 | 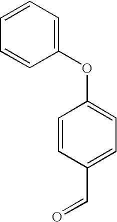 | 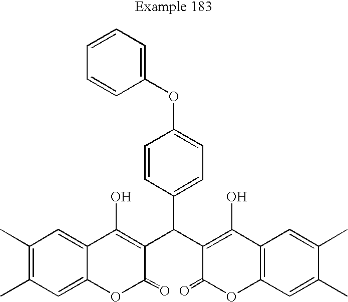 | 81.5 | 561.2[M + H]⁺ |
| 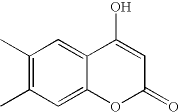 | 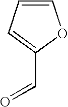 | 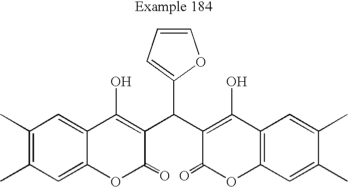 | 30.9 | 459.1[M + H]⁺ |
| 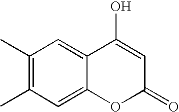 | 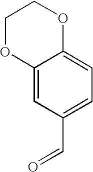 | 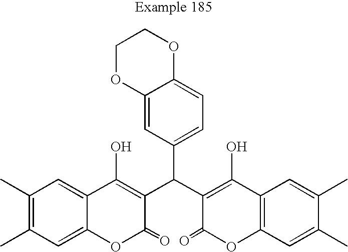 | 77.9 | 527.1[M + H]⁺ |
| 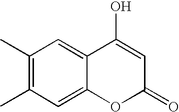 | 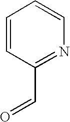 | 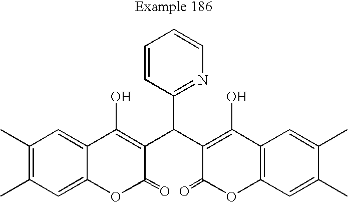 | 54.2 | 470.3[M + H]⁺ |
| 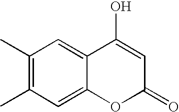 | 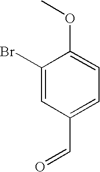 | 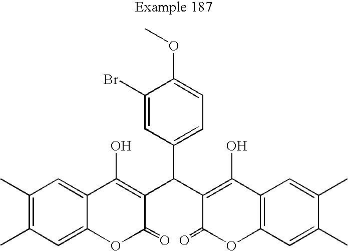 | 78.3 | 577.1[M + H]⁺ |
| 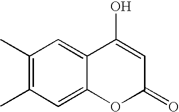 | 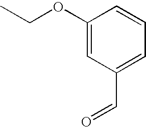 | 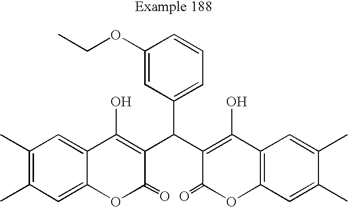 | 66.7 | 513.2[M + H]⁺ |
| 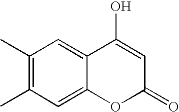 | 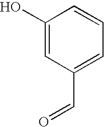 | 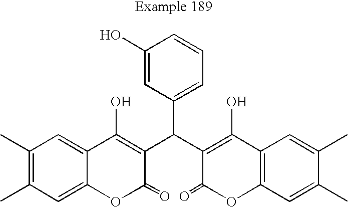 | 47.0 | 485.2[M + H]⁺ |
| 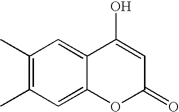 | 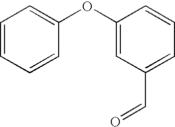 | 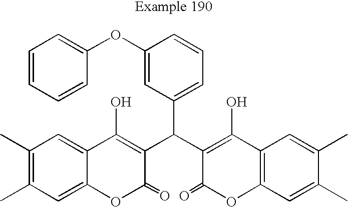 | 20.8 | 561.2[M + H]⁺ |
| 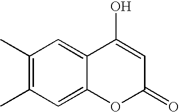 | 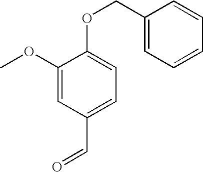 | 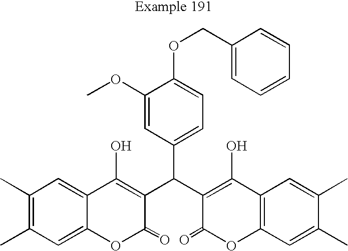 | 59.4 | 605.2[M + H]⁺ |
| 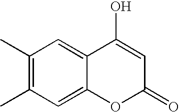 | 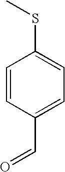 | 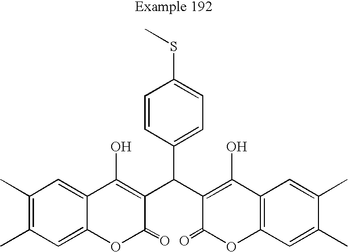 | 78.0 | 515.1[M + H]⁺ |
| 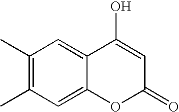 | 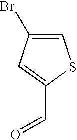 | 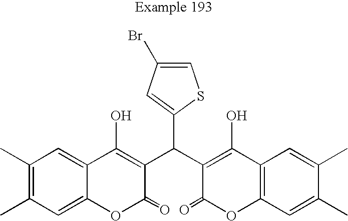 | 35.5 | 553.0[M + H]⁺ |
| 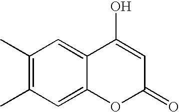 | 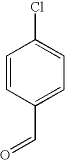 | 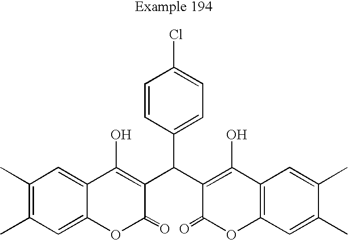 | 44.0 | 503.1[M + H]⁺ |
| 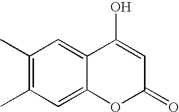 | 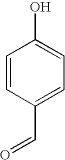 | 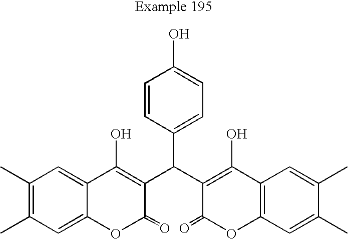 | 79.7 | 485.1[M + H]⁺ |
| 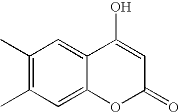 | 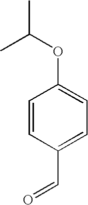 | 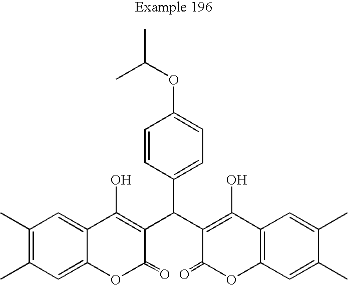 | 33.9 | 527.2[M + H]⁺ |
| 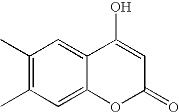 | 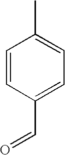 | 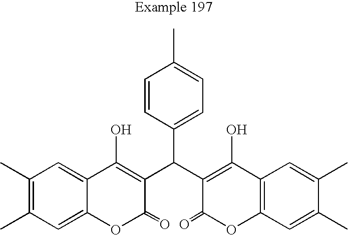 | 55.6 | 483.2[M + H]⁺ |
| 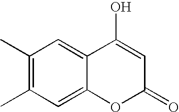 | 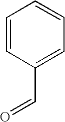 | 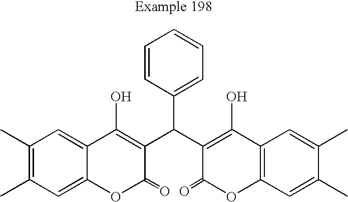 | 34.9 | 469.2[M + H]⁺ |
| 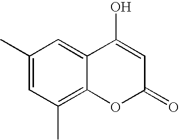 | 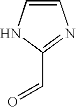 | 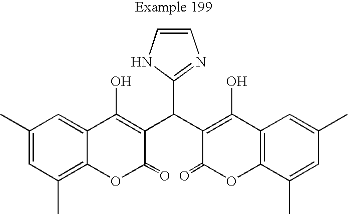 | 88.6 | 458.9[M + H]⁺ |
| 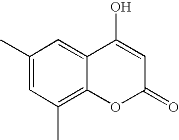 | 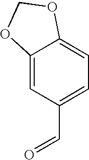 | 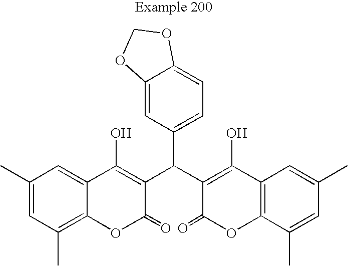 | 84.2 | 513.0[M + H]⁺ |
| 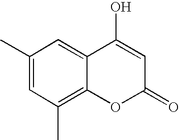 | 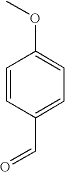 | 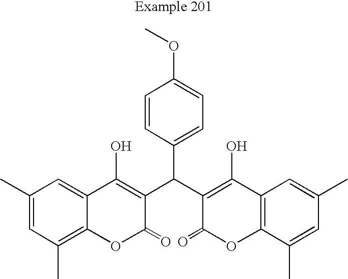 | 93.4 | 499.0[M + H]⁺ |
| 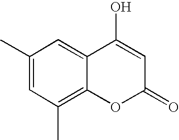 | 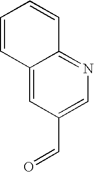 | 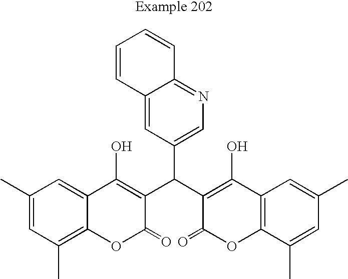 | 76.7 | 520.0[M + H]⁺ |
| 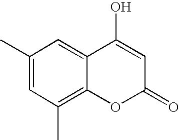 | 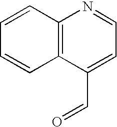 | 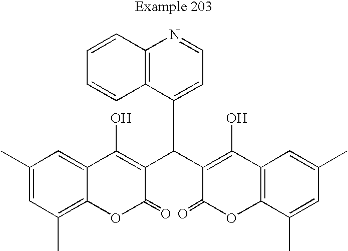 | 86.2 | 520.0[M + H]⁺ |
| 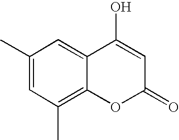 | 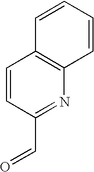 | 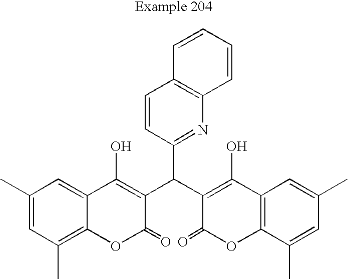 | 81.6 | 520.0[M + H]⁺ |
| 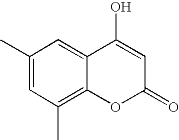 | 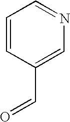 | 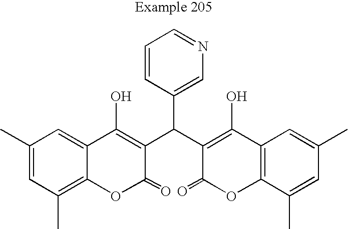 | 13.5 | 470.1[M + H]⁺ |
| 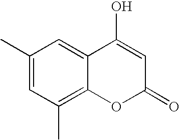 | 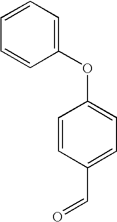 | 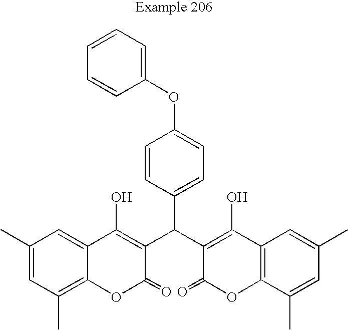 | 86.5 | 561.0[M + H]⁺ |
| 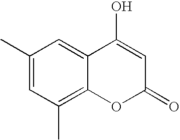 | 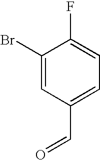 | 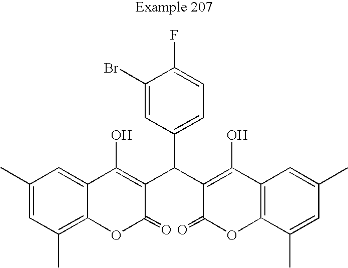 | 47.5 | 564.9[M + H]⁺ |
| 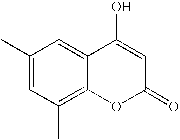 | 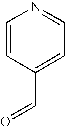 | 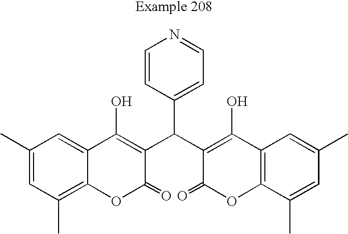 | 87.4 | 469.8[M + H]⁺ |
| 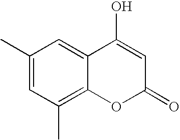 | 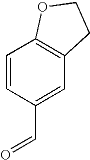 | 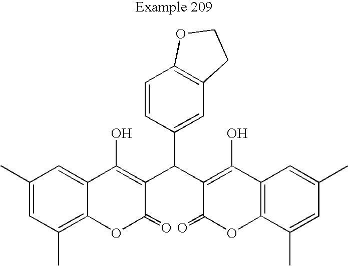 | 85.5 | 510.9[M + H]⁺ |
| 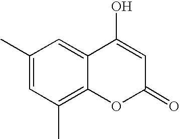 | 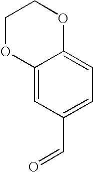 | 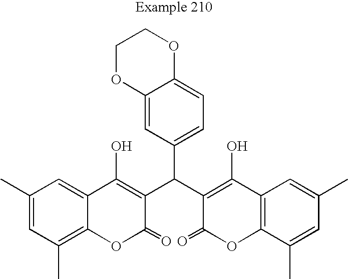 | 93.3 | 525.0[M − H]⁻ |
| 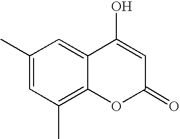 | 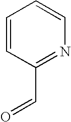 | 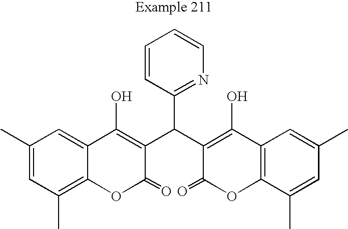 | 74.7 | 470.0[M + H]⁺ |
| 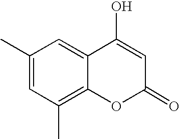 | 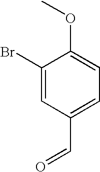 | 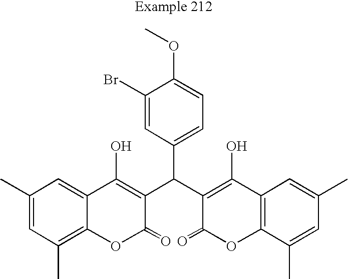 | 71.9 | 574.8[M − H]⁻ |
| 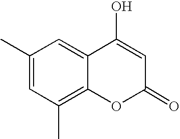 | 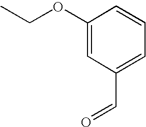 | 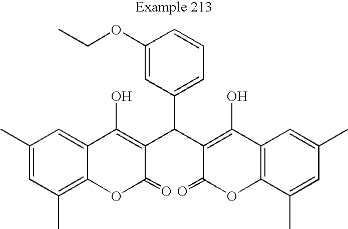 | 79.5 | 511.1[M − H]⁻ |
| 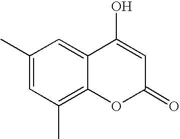 | 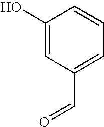 | 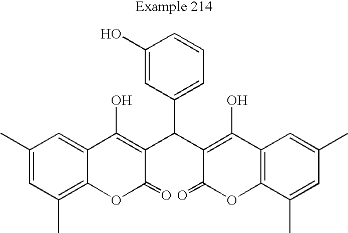 | 67.1 | 483.0[M − H]⁻ |
| 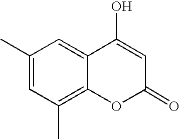 | 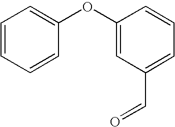 | 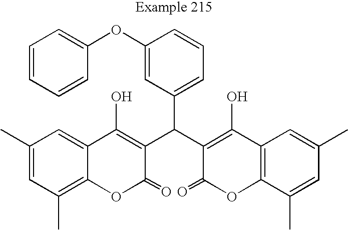 | 56.7 | 559.0[M − H]⁻ |
| 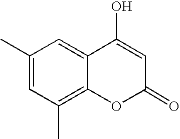 | 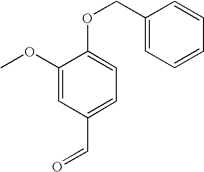 | 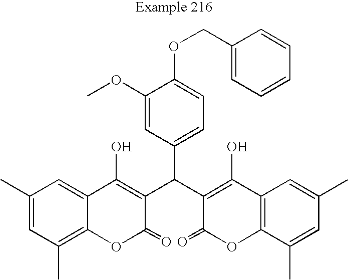 | 81.4 | 603.0[M − H]⁻ |
| 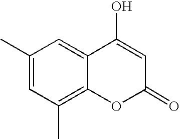 | 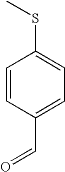 | 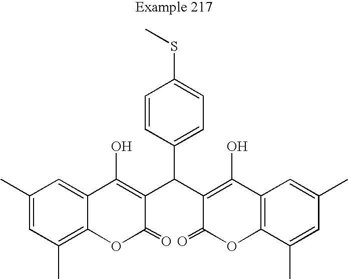 | 95.7 | 513.0[M − H]⁻ |
| 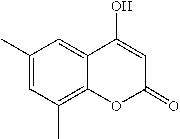 | 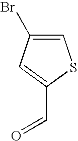 | 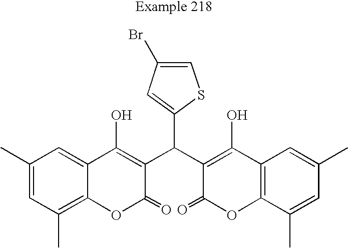 | 21.0 | 550.7[M − H]⁻ |
| 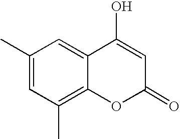 | 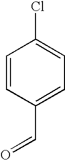 | 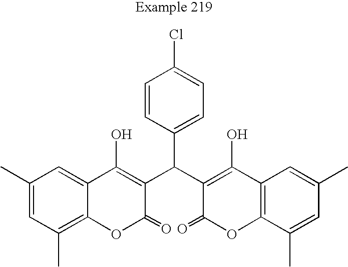 | 88.8 | 500.9[M − H]⁻ |
| 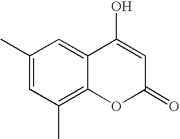 | 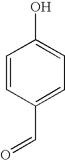 | 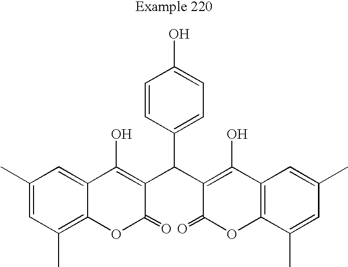 | 87.8 | 482.9[M − H]⁻ |
| 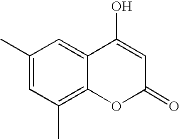 | 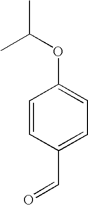 | 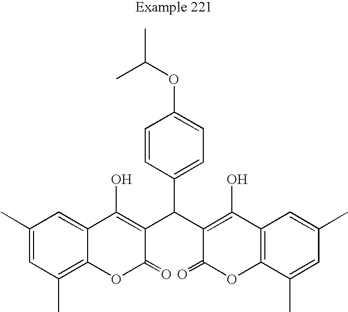 | 85.1 | 525.0[M − H]⁻ |
| 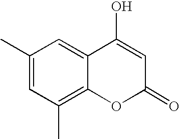 | 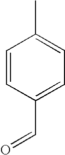 | 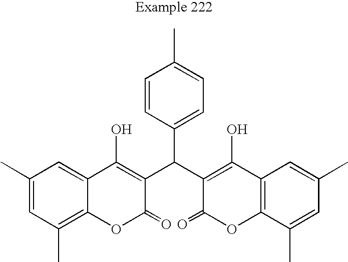 | 87.6 | 481.0[M − H]⁻ |
| 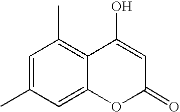 | 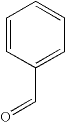 | 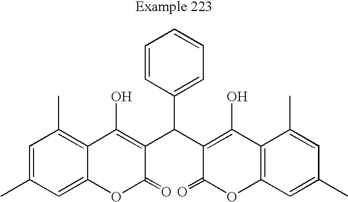 | 33.1 | 469.2[M + H]⁺ |
| 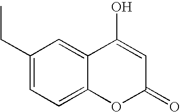 | 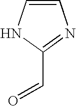 | 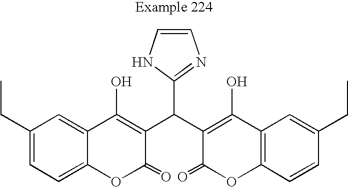 | 99.9 | 458.9[M + H]⁺ |
| 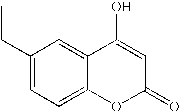 | 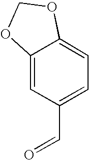 | 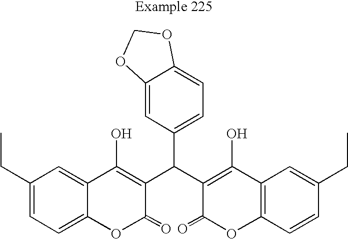 | 79.8 | 511.1[M − H]⁻ |
| 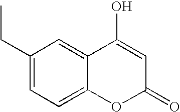 | 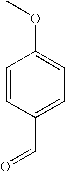 | 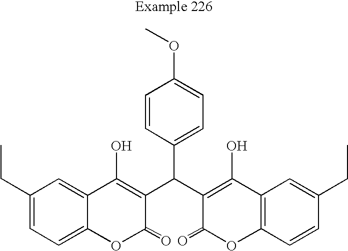 | 80.5 | 497.1[M − H]⁻ |
| 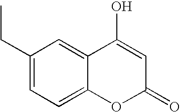 | 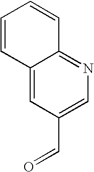 | 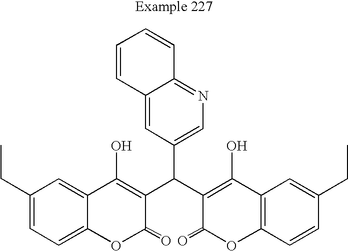 | 61.9 | 518.1[M − H]⁻ |
| 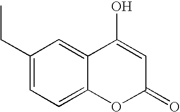 | 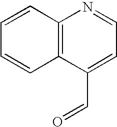 | 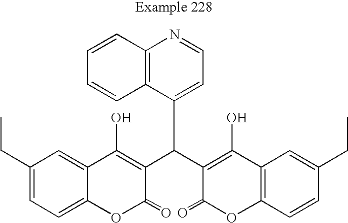 | 39.7 | 518.1[M − H]⁻ |
| 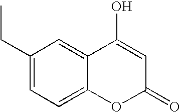 | 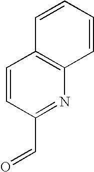 | 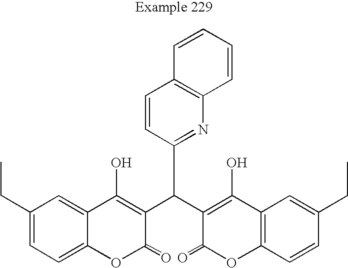 | 69.2 | 520.6[M + H]⁺ |
| 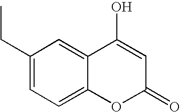 | 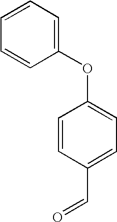 | 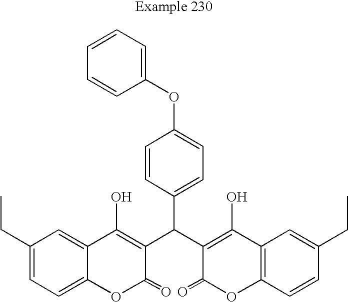 | 83.5 | 559.2[M − H]⁻ |
| 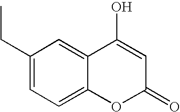 | 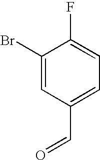 | 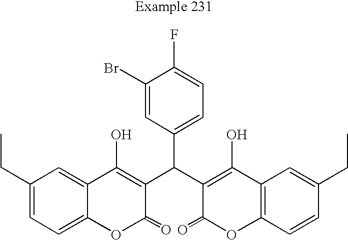 | 22.4 | 563.0[M − H]⁻ |
| 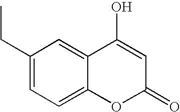 | 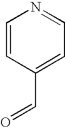 | 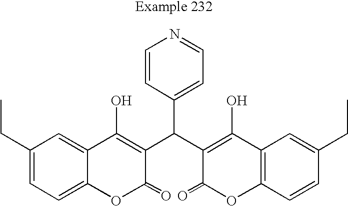 | 82.8 | 468.0[M − H]⁻ |
| 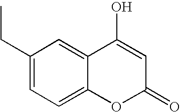 | 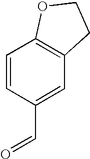 | 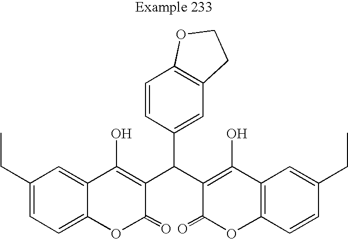 | 70.7 | 532.8[M + Na]⁺ |
| 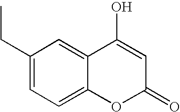 | 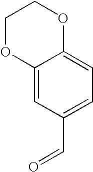 | 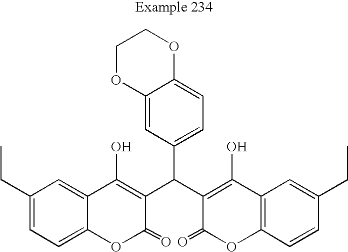 | 82.5 | 526.9[M + H]⁺ |
| 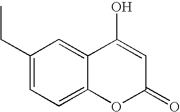 | 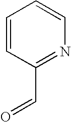 | 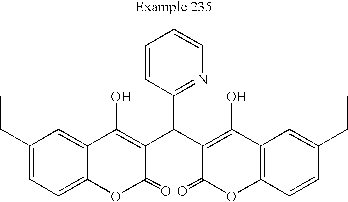 | 73.9 | 469.7[M + H]⁺ |
| 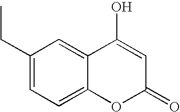 | 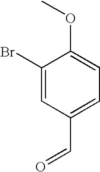 | 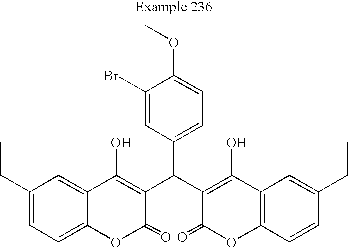 | 75.0 | 576.8[M + H]⁺ |
| 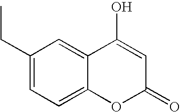 | 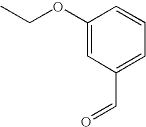 | 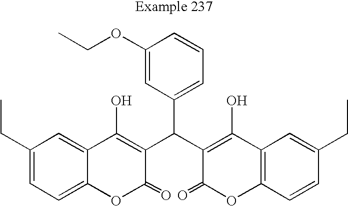 | 78.8 | 534.9[M + Na]⁺ |
| 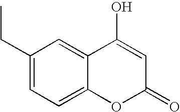 | 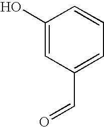 | 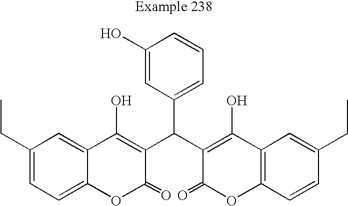 | 78.1 | 483.1[M − H]⁻ |
| 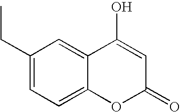 | 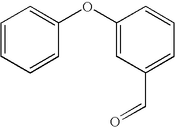 | 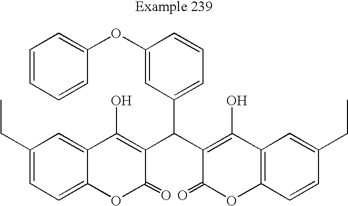 | 46.7 | 560.9[M + H]⁺ |
| 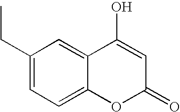 | 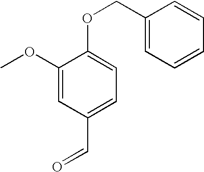 | 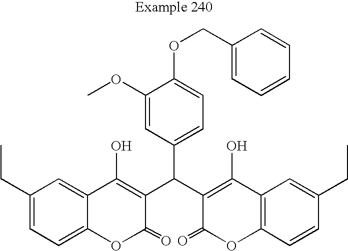 | 67.0 | 603.0[M − H]⁻ |
| 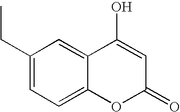 | 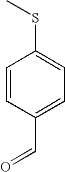 | 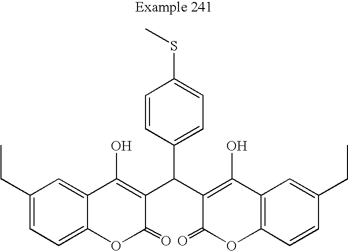 | 74.0 | 536.7[M + Na]⁺ |
| 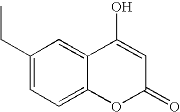 | 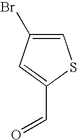 | 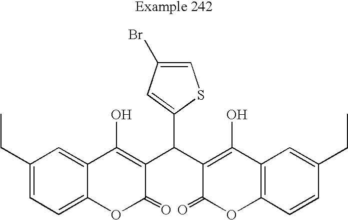 | 11.9 | 550.9[M − H]⁻ |
| 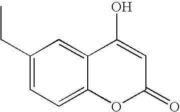 | 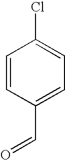 | 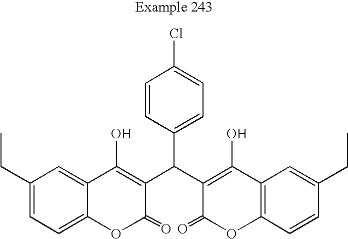 | 39.3 | 501.0[M − H]⁻ |
| 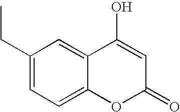 | 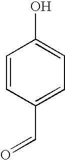 | 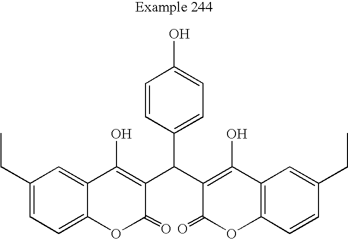 | 21.4 | 483.1[M − H]⁻ |
| 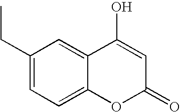 | 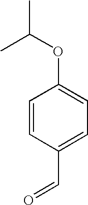 | 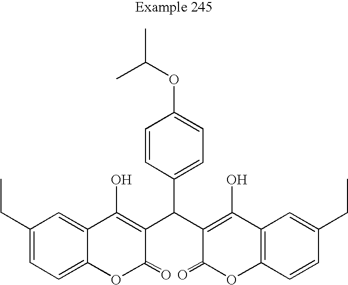 | 80.0 | 549.0[M + Na]⁺ |
| 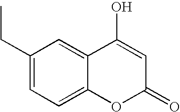 | 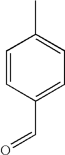 | 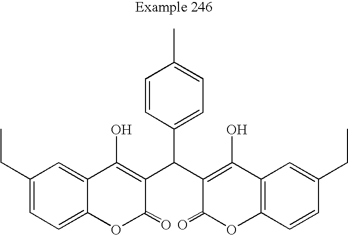 | 72.9 | 481.1[M − H]⁻ |
| 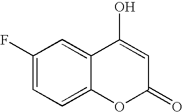 | 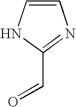 | 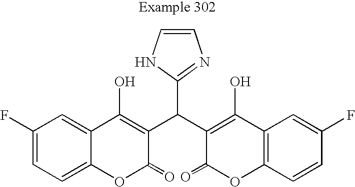 | 69.4 | 439.3[M + H]⁺ |
| 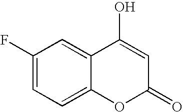 | 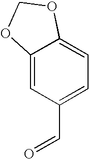 | 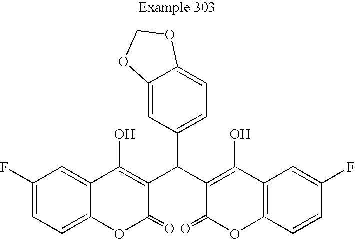 | 67.1 | 490.9[M − H]⁻ |
| 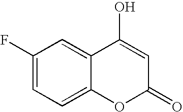 | 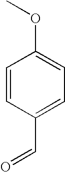 | 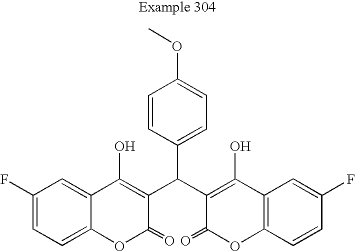 | 64.1 | 476.9[M − H]⁻ |
| 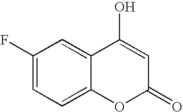 | 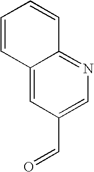 | 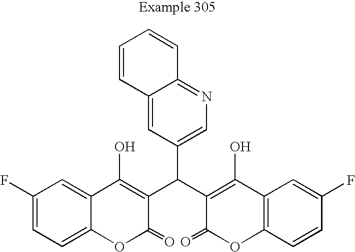 | 63.5 | 499.8[M + H]⁺ |
| 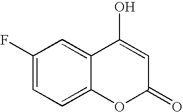 | 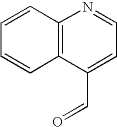 | 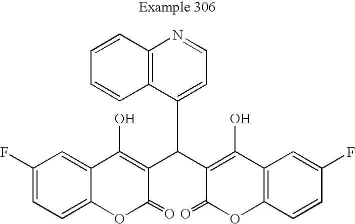 | 66.2 | 500.3[M + H]⁺ |
| 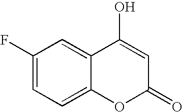 | 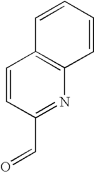 | 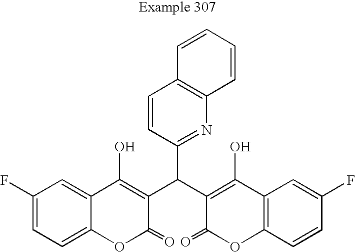 | 65.9 | 499.7[M + H]⁺ |
| 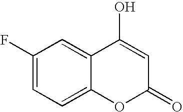 | 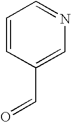 | 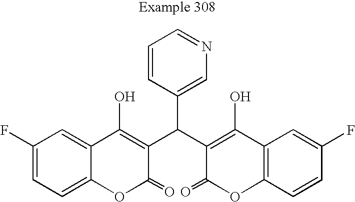 | 69.0 | 449.6[M + H]⁺ |
| 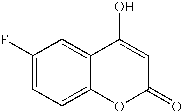 | 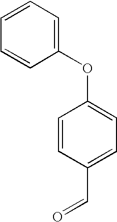 | 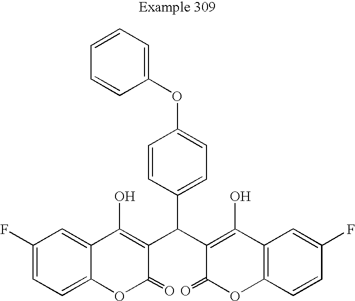 | 67.8 | 539.0[M − H]⁻ |
| 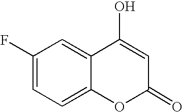 | 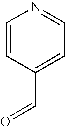 | 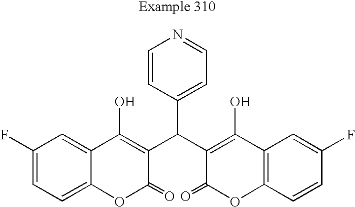 | 65.3 | 450.6[M + H]⁺ |
| 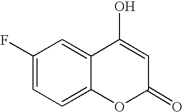 | 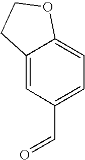 | 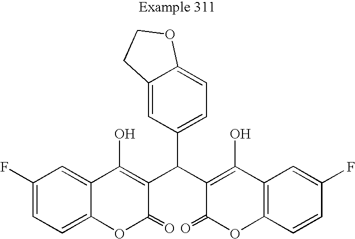 | 61.5 | 488.9[M − H]⁻ |
| 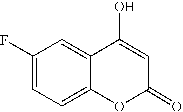 | 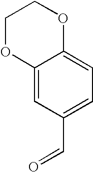 | 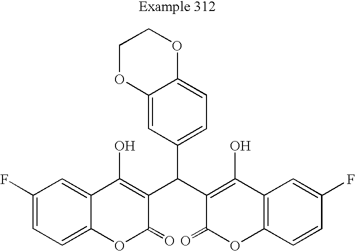 | 61.0 | 504.9[M − H]⁻ |
| 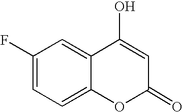 | 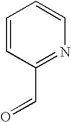 | 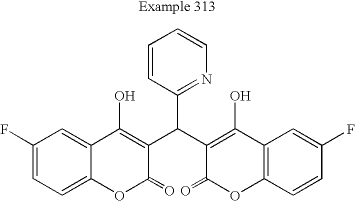 | 57.0 | 449.7[M + H]⁺ |
| 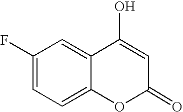 | 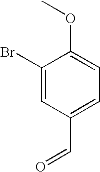 | 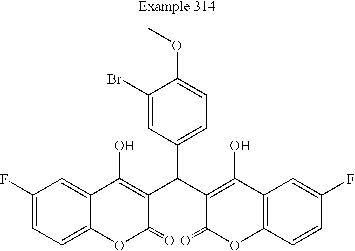 | 78.0 | 554.8[M − H]⁻ |
| 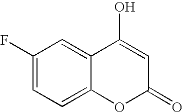 | 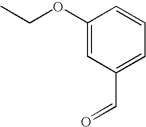 | 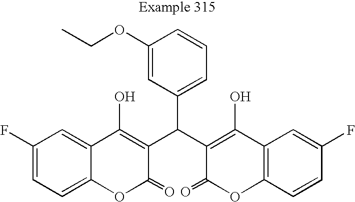 | 19.5 | 490.9[M − H]⁻ |
| 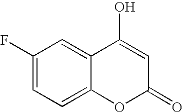 | 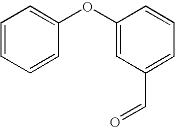 | 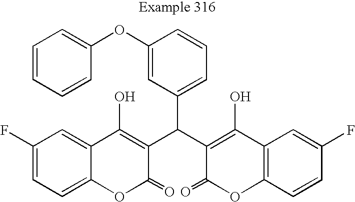 | 67.6 | 539.0[M − H]⁻ |
| 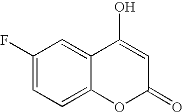 | 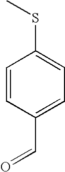 | 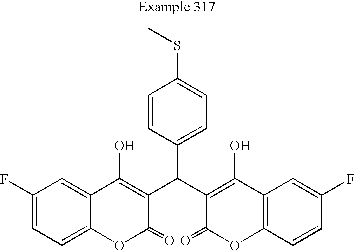 | 70.5 | 492.9[M − H]⁻ |
| 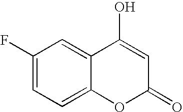 | 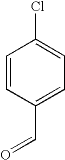 | 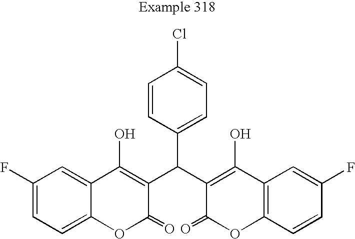 | 37.7 | 480.9[M − H]⁻ |
| 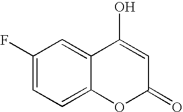 | 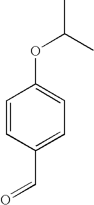 | 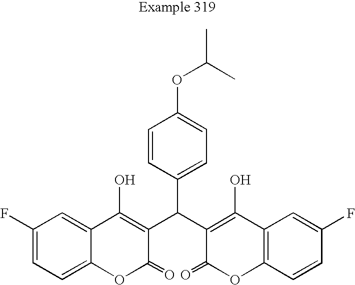 | 63.4 | 504.9[M − H]⁻ |
| 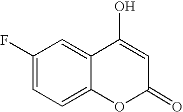 | 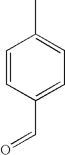 | 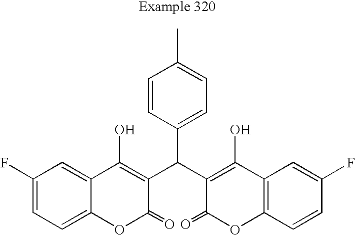 | 76.1 | 460.9[M − H]⁻ |
| 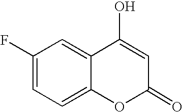 | 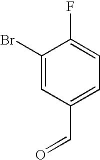 | 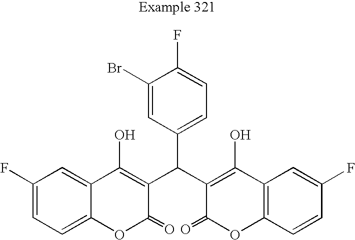 | 43.5 | 542.8[M − H]⁻ |
| 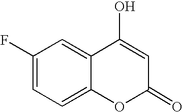 | 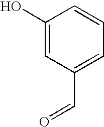 | 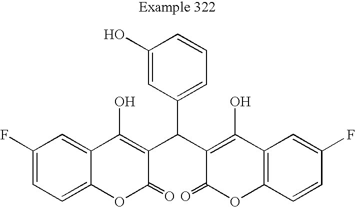 | 55.7 | 462.9[M − H]⁻ |
| 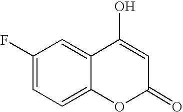 | 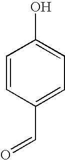 | 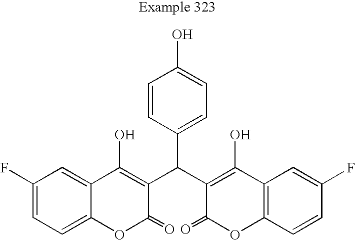 | 41.5 | 462.9[M − H]⁻ |
| 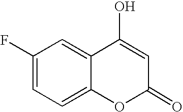 | 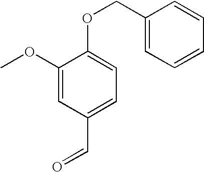 | 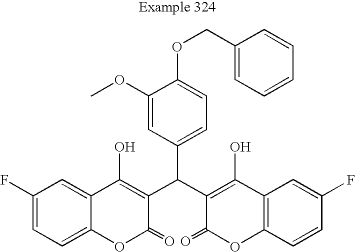 | 70.5 | 584.9[M + H]⁺ |
| 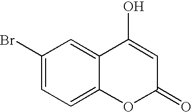 | 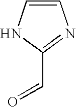 | 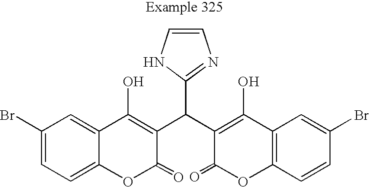 | 74.6 | 558.4[M + H]⁺ |
| 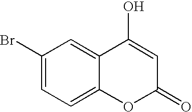 | 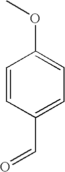 | 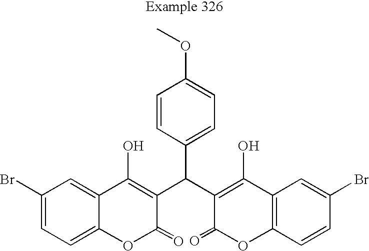 | 76.3 | 596.6[M − H]⁻ |
| 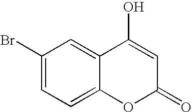 | 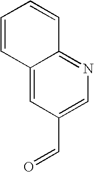 | 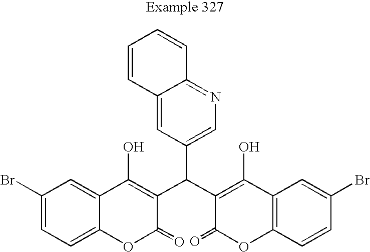 | 57.4 | 617.7[M − H]⁻ |
| 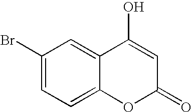 | 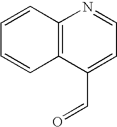 | 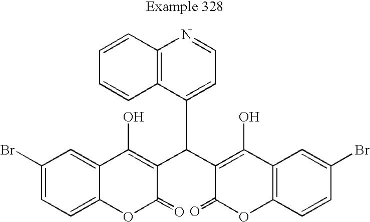 | 90.9 | 617.8[M − H]⁻ |
| 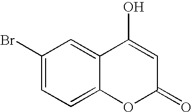 | 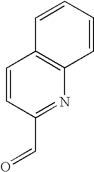 | 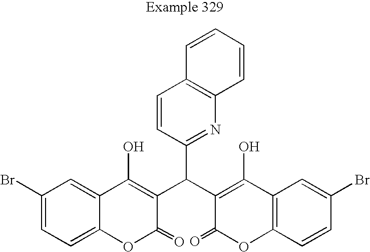 | 82.0 | 617.8[M − H]⁻ |
| 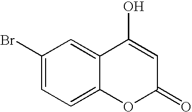 | 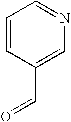 | 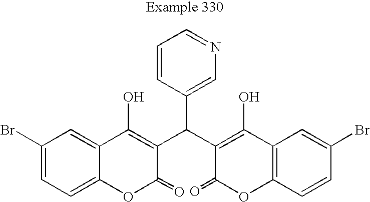 | 72.0 | 567.7[M − H]⁻ |
| 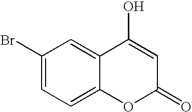 | 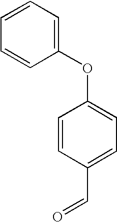 | 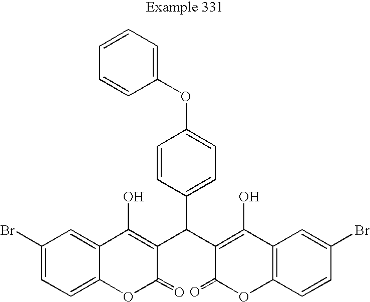 | 84.0 | 658.7[M − H]⁻ |
| 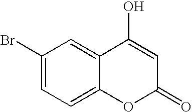 | 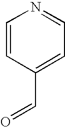 | 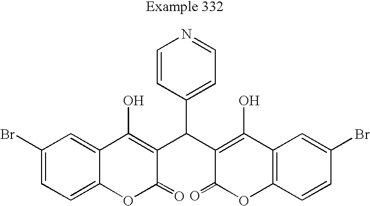 | 76.2 | 567.6[M − H]⁻ |
| 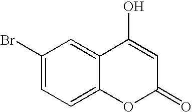 | 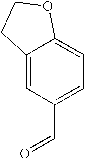 | 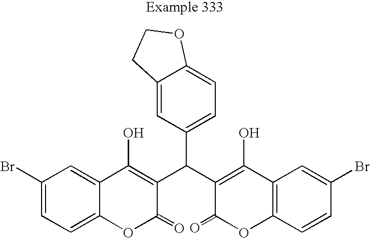 | 82.7 | 608.7[M − H]⁻ |
| 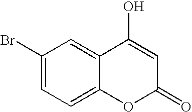 | 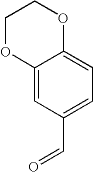 | 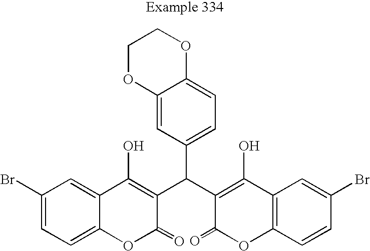 | 75.5 | 624.6[M − H]⁻ |
| 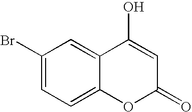 | 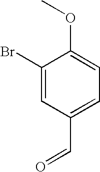 | 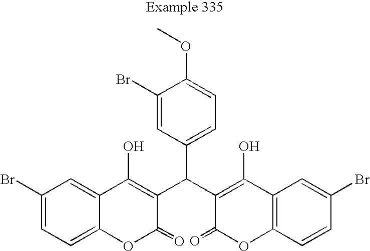 | 86.9 | 674.6[M − H]⁻ |
| 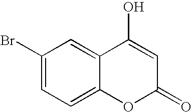 | 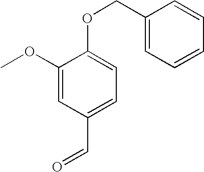 | 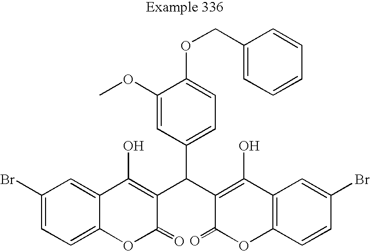 | 74.4 | 702.8[M − H]⁻ |
| 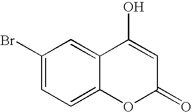 | 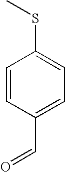 | 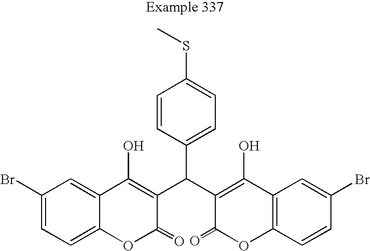 | 83.2 | 612.7[M − H]⁻ |
| 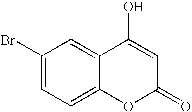 | 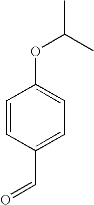 | 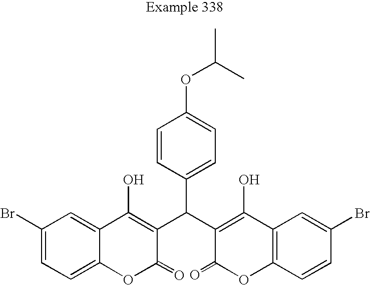 | 48.0 | 624.7[M − H]⁻ |
| 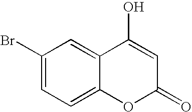 | 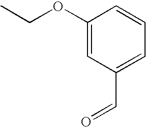 | 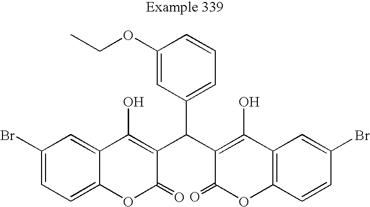 | 32.3 | 610.7[M − H]⁻ |
| 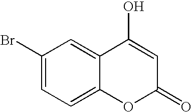 | 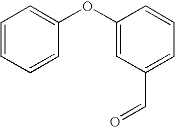 | 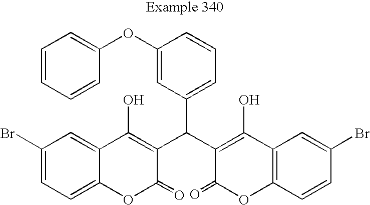 | 21.2 | 658.7[M − H]⁻ |
| 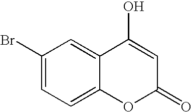 | 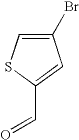 | 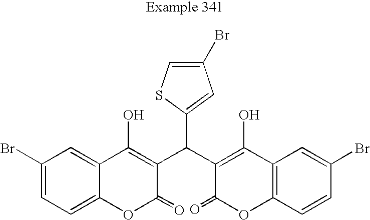 | 17.7 | 650.5[M − H]⁻ |
| 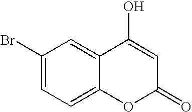 | 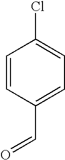 | 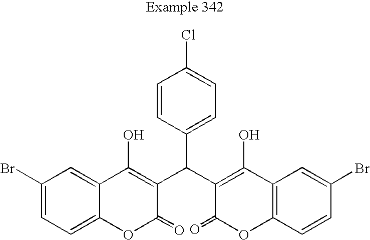 | 13.1 | 600.6[M − H]⁻ |
| 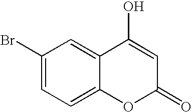 | 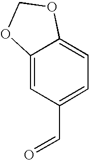 | 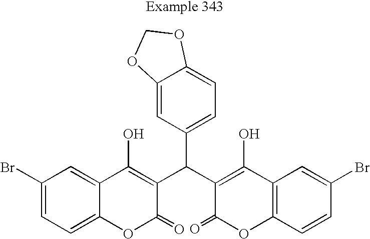 | 80.4 | 610.6[M − H]⁻ |
| 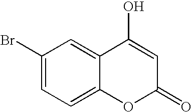 | 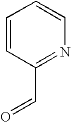 | 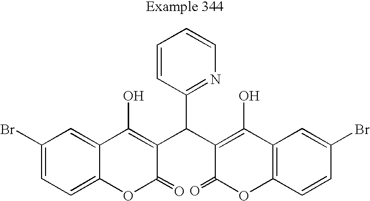 | 73.7 | 567.7[M − H]⁻ |
| 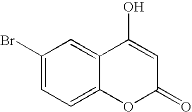 | 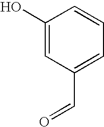 | 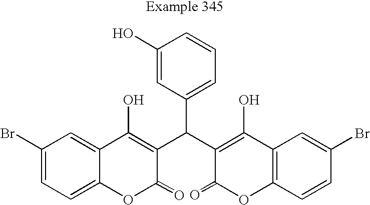 | 64.9 | 582.7[M − H]⁻ |
| 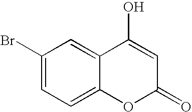 | 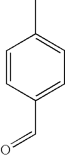 | 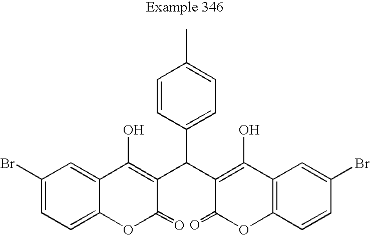 | 74.0 | 580.6[M − H]⁻ |
| 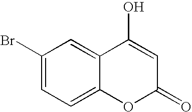 | 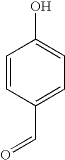 | 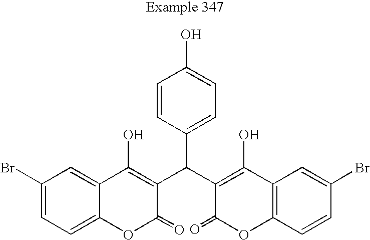 | 43.7 | 582.6[M − H]⁻ |
| 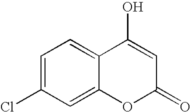 | 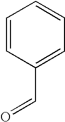 | 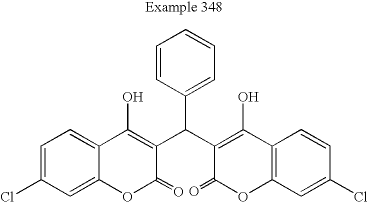 | 80.5 | 479.0[M − H]⁻ |
| 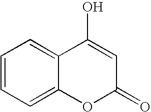 | 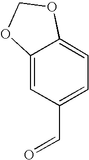 | 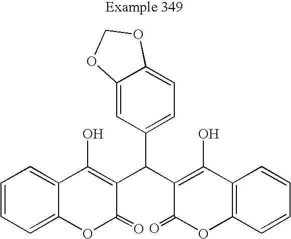 | 85.7 | 457.2[M + H]⁺ |
| 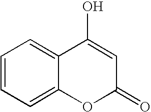 | 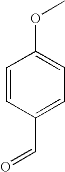 | 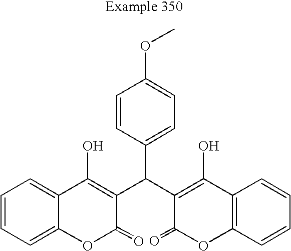 | 83.1 | 443.2[M + H]⁺ |
| 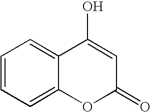 | 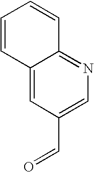 | 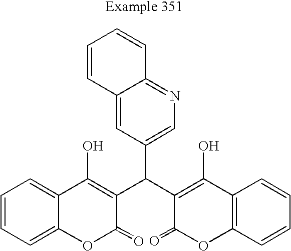 | 77.5 | 464.3[M + H]⁺ |
| 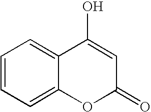 | 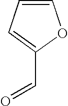 | 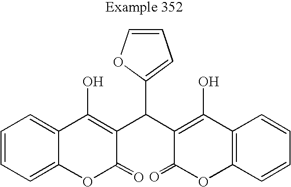 | 91.3 | 403.2[M + H]⁺ |
| 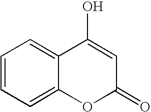 | 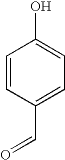 | 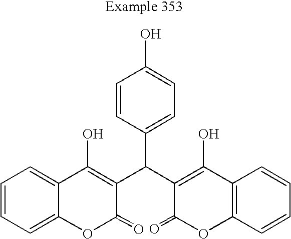 | 82.1 | 429.2[M + H]⁺ |
| 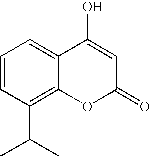 | 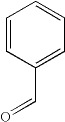 | 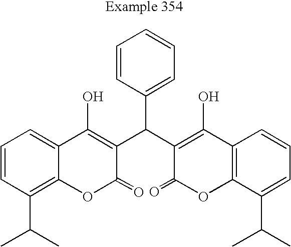 | 72.0 | 497.4[M + H]⁺ |
| 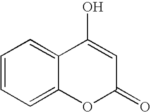 | 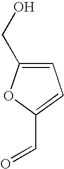 | 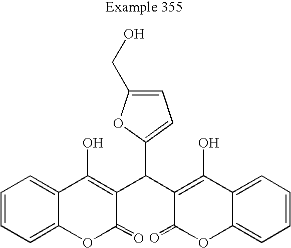 | 55.0 | 430.9[M − H]⁻ |
| 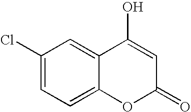 | 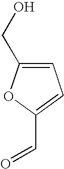 | 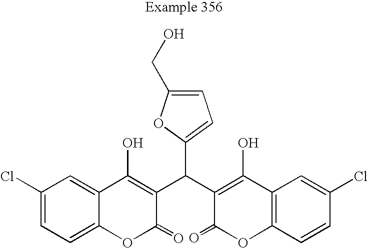 | 22 | 498.8[M − H]⁻ |
| 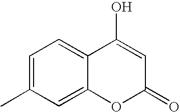 | 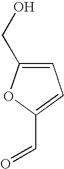 | 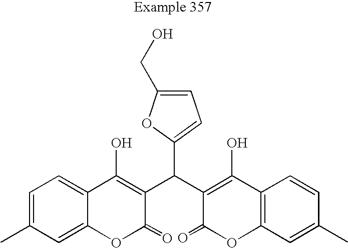 | 7.6 | 459.0[M − H]⁻ |
| 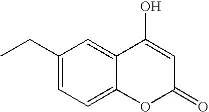 | 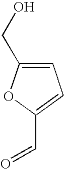 | 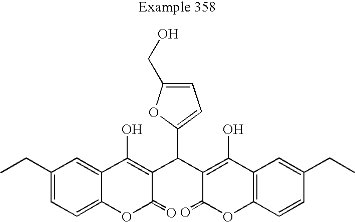 | 25 | 486.9[M − H]⁻ |
| 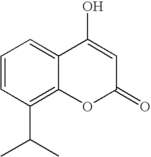 | 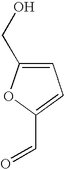 | 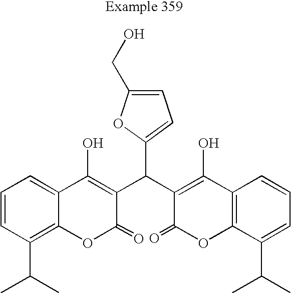 | 20 | 515.0[M − H]⁻ |

### Examples from 360 to 365

### General procedure for the preparation of compounds of Formula I CH₃

wherein A represents a CH—X group, X is a

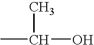

group and n=0

To a solution of Na(CN)BH₃ (2 mmol) in MeOH (5 mL) previously acidified with 1 M aq. HCl corresponding compound of Formula I, wherein A represents a CH—X group, X is an acetyl group and n=0 (1 mmol) was added. Reaction mixture was heated to reflux and stirring under reflux was continued for 0.5-6 h. If necessary, additional portion of reducing agent (1 mmol) was added into the reaction mixture, followed by 1 M aq. HCl in MeOH to maintain the reaction mixture acidic, and reflux was continued for next 0.5-6 h. After cooling to room temperature, solvent was evaporated and dry residue was dissolved in water (50 mL), followed by extraction with EtOAc (3×50 mL) with addition of saturated aq. NaCl. Organic layers were combined, solvent was evaporated and the residue was dissolved in water (30 mL), followed by acidification with 1 M aq. HCl until pH=1 resulting in precipitation of the product, which was then filtered off and dried.

| | | Yield | MS |
|---|---|---|---|
| Starting compound | Product | (%) | (ESI, m/z) |
| 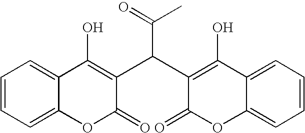 | 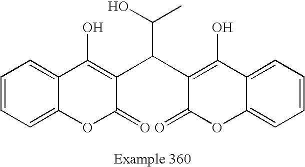 | 78 | 378.9[M − H]⁻ |
| 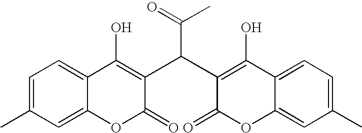 | 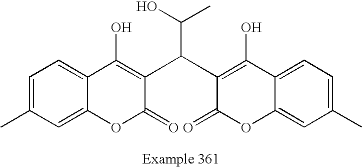 | 73 | 407.0[M − H]⁻ |
| 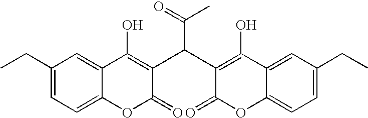 | 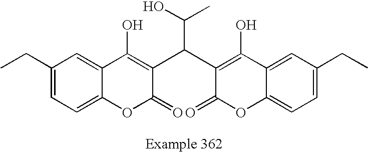 | 71 | 436.9[M + H]⁺ |
| 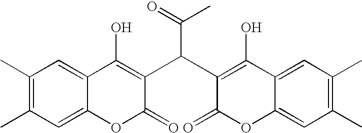 | 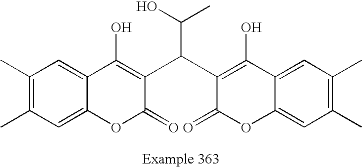 | 62 | 459.2[M + Na]⁺ |
| 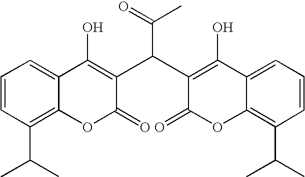 | 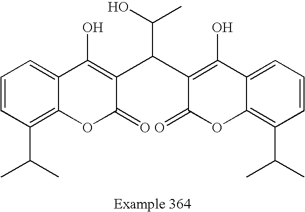 | 85 | 465.0[M + H]⁺ |
| 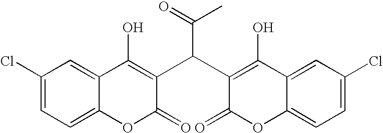 | 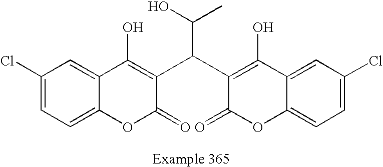 | 42 | 446.9[M − H]⁻ |

### Examples from 247 to 250

### General Procedure for the Preparation of Compounds of Formula II

wherein X is carboxy

To a solution of corresponding ester of Formula I, wherein A represents a CH—X group, X is an alkyloxycarbonyl group and n=0, in Py was added Ac₂O, Stirring of the reaction mixture at room temperature was continued overnight, precipitate was filtered off, washed with n-hexane and dried. Corresponding ester of Formula II, wherein X is alkyloxycarbonyl group, was thus formed, which was in the next step subjected to hydrolysis of the ester group as described bellow.

To a suspension of compound of Formula II, wherein X is alkyloxycarbonyl group, in THF, 1M aq. NaOH was added. Stirring was continued at room temperature overnight or until the completion of the reaction. Reaction mixture was then acidified by 2M aq. HCl, and stirring at 0° C. continued for a while resulting in the formation of precipitate, which was filtered off, washed with ether and dried.

| | | | MS |
|---|---|---|---|
| Starting compound | Product | Yield (%) | (ESI, m/z) |
| 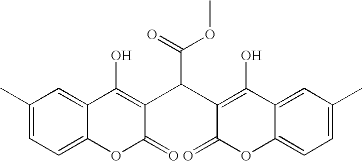 | 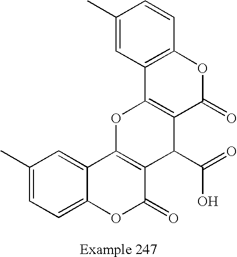 | 49 | 412.7[M + Na]⁺ |
| 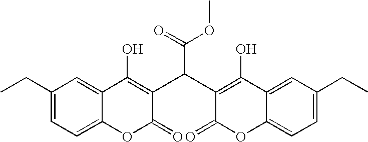 | 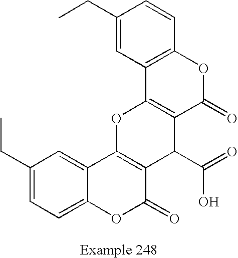 | 70 | 419.1[M + H]⁺ |
| 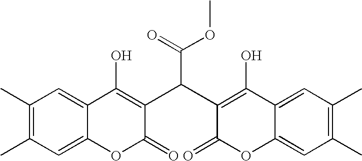 | 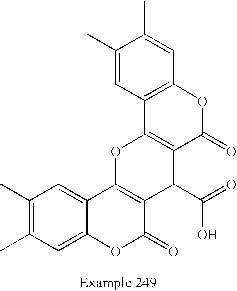 | 56 | 440.7[M + Na]⁺ |
| 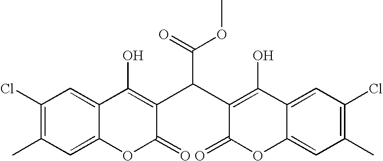 | 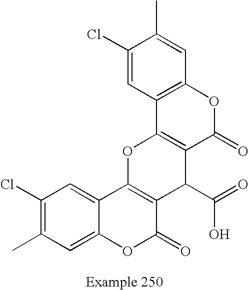 | 77 | 459.0[M + H]⁺ |

### Examples from 251 to 254

### General Procedure for the Preparation of Compounds of Formula II

wherein X is an alkylcarbonyl group

A solution of corresponding ketone of Formula I, wherein A represents aCH-X group, X is an alkylcarbonyl group and n=0, in Ac₂O was heated to reflux, and stirred under reflux until completion of the reaction (2-10 h). After cooling to room temperature, precipitate was filtered off, washed with cold n-hexane and dried.

| | | | MS |
|---|---|---|---|
| Starting compound | Product | Yield (%) | (ESI, m/z) |
| 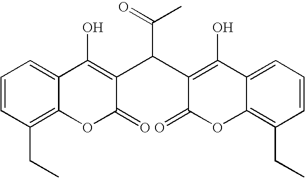 | 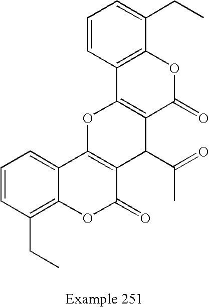 | 42 | 417.2[M + H]⁺ |
| 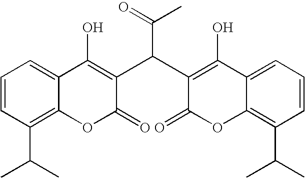 | 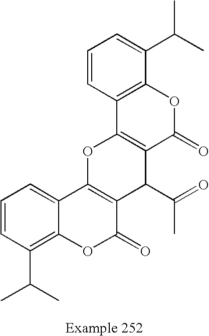 | 33 | 445.0[M + H]⁺ |
| 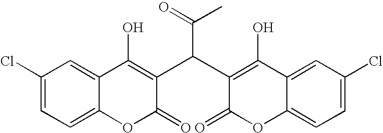 | 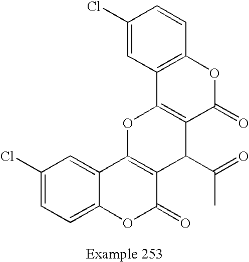 | 53 | 429.1[M + H]⁺ |
| 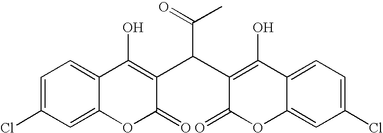 | 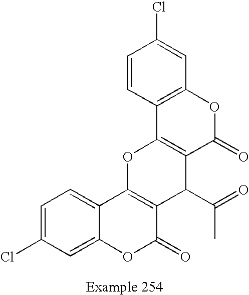 | 41 | 429.0[M + H]⁺ |

### Examples from 255 to 258

### General Procedure for the Preparation of Compounds of Formula II

wherein X is a formyl group

A solution of corresponding intramolecular hemiacetal of Formula III, wherein D represents a CHOH group and —— denotes a single bond (See, e.g., WO 2005/010006), in Ac₂O was heated to reflux, and stirring under reflux was continued until completion of the reaction (4-8 h). After cooling to room temperature, precipitate was filtered off, washed with ether and dried. Corresponding acetyl derivative of the enol form of compound of Formula II, wherein X is formyl group, was thus formed, which was in the next step subjected to hydrolysis of the acetyl group as described bellow.

To a solution of acetyl derivative of the enol form of compound of Formula II, wherein X is formyl group, in AcOH was added water in small portions until solution turned into suspension. The reaction mixture was heated to reflux, and stirring under reflux was continued until completion of the reaction (12-48 h). After cooling to room temperature, precipitate was filtered off, washed with ether and dried.

| | | | MS |
|---|---|---|---|
| Starting compound | Product | Yield (%) | (ESI, m/z) |
| 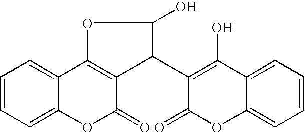 | 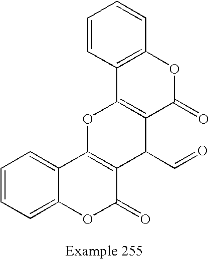 | 32 | 346.9[M + H]⁺ |
| 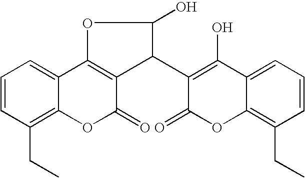 | 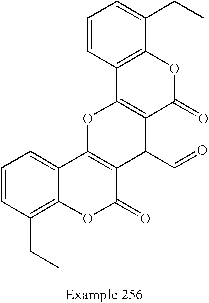 | 12 | 403.1[M + H]⁺ |
| 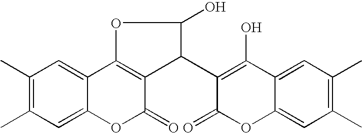 | 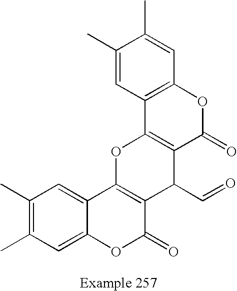 | 16 | 403.1[M + H]⁺ |
| 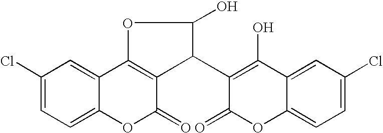 | 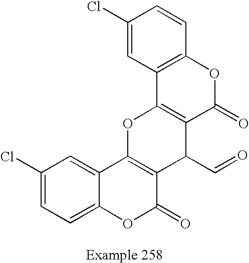 | 16 | 415.1[M + H]⁺ |

### Examples from 259 to 261 and 366

### General Procedures for the Preparation of Compounds of Formula II

wherein X is

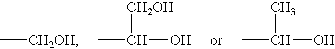

group

Method A

A solution of corresponding compound of Formula I, wherein A represents CH—X group, X is

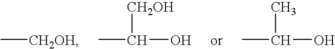

group and n=0, in Ac₂O was heated to reflux, and stirred under reflux until completion of the reaction (0.1-10 h). After cooling to room temperature, precipitate was filtered off, washed with cold n-hexane and dried.

Method B

To a solution of corresponding compound of Formula I, wherein A represents CH—X group, X is

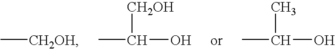

group and n=0, in Py was added Ac₂O. The reaction mixture was stirred at room temperature overnight or until completion of the reaction. Precipitate was filtered off, washed with n-hexane and dried.

Method C

A solution of corresponding compound of Formula I, wherein A represents CH—X group, X is

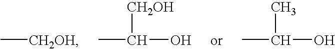

group and n=0, in Ac₂O was heated to reflux until suspension was formed, followed by addition of pyridine until formation of the solution. Stirring under reflux was continued until completion of the reaction (0.2-2 h). After cooling to room temperature, precipitate was filtered off, washed with cold n-hexane and dried.

General Deacetylation Procedure of Acetylated Derivatives of Formula II, Obtained by Methods A, B or C

To a suspension of corresponding acetylated derivative of Formula II in 1,4-dioxane was added 1M aq. KOH. Stirring was continued at room temperature overnight or until the completion of the reaction. Reaction mixture was then acidified by 2M aq. HCl and organic solvent removed under reduced pressure, followed by addition of water. Precipitate was filtered off, triturated or recrystallized from MeOH and dried.

| | | | MS |
|---|---|---|---|
| Starting compound | Product | Yield (%) | (ESI, m/z) |
| 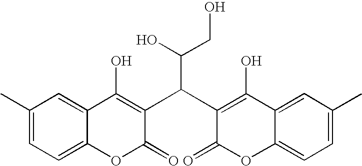 | 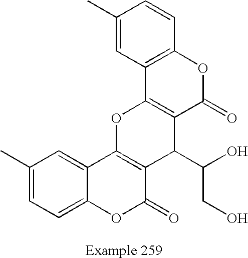 | 18(Method A) | 407.3[M + H]⁺ |
| 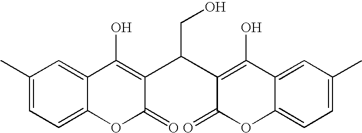 | 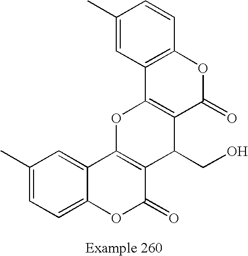 | 12(Method A)14(Method B) | 376.7[M + H]⁺ |
| 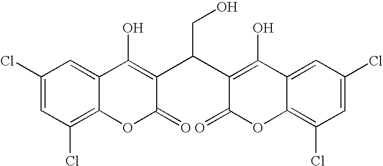 | 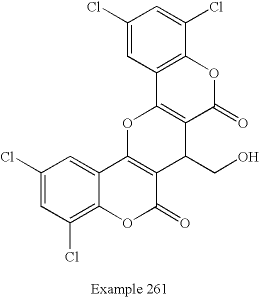 | 18(Method C) | 484.6[M + H]⁺ |
| 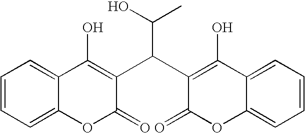 | 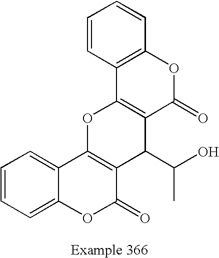 | 68 | 363.1[M + H]⁺ |

### Examples from 262 to 272

### General Procedures for the Preparation of Compounds of Formula III

wherein D represents carbonyl

Method A

A solution of corresponding compound of Formula I, wherein A represents CH—X,

wherein X is a carboxy group and n=0, in SOCl₂ was heated to reflux, and stirred under reflux until completion of the reaction (0.05-1 h). After cooling to room temperature, stirring of the reaction mixture was continued, followed by addition of CH₂Cl₂ and/or n-hexane until precipitation occurred. Precipitate was filtered off, washed with cold n-hexane and dried.

Method B

To a suspension of corresponding compound of Formula I, wherein A represents CH—X, wherein X is a carboxy group and n=0, in AcOH was added SOCl₂, and the reaction mixture was heated to 80° C. Stirring at 80° C. was continued for several hours. Reaction mixture was cooled to room temperature, precipitate was filtered off, washed with AcOH, followed by n-hexane and dried.

Method C

To a suspension of corresponding compound of Formula I, wherein A represents CH—X, wherein X is a carboxy group and n=0, in AcOH was added Ac₂O, and the reaction mixture was heated to reflux. Stirring under reflux was continued for several hours. Reaction mixture was cooled to +4° C., precipitate was filtered off, suspended in AcOH, heated to reflux and stirring under reflux was continued for an hour. After cooling to room temperature, precipitate was filtered off, washed with ether and dried.

| | | Yield | MS |
|---|---|---|---|
| Starting compound | Product | (%) | (ESI, m/z) |
| 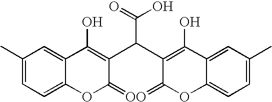 | 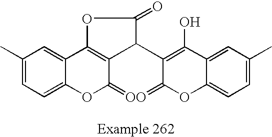 | 83(Method A)59(Method C) | 389.2[M − H]⁻ |
| 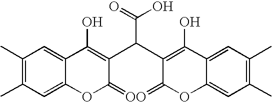 | 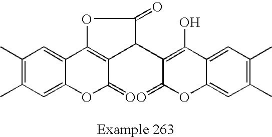 | 78(Method A) | 419.2[M + H]⁺ |
| 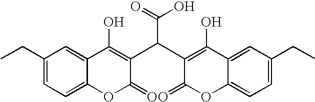 | 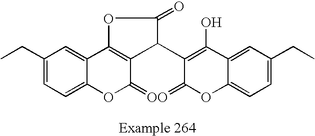 | 67(Method B) | 419.2[M + H]⁺ |
| 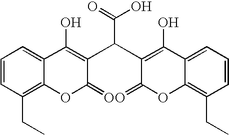 | 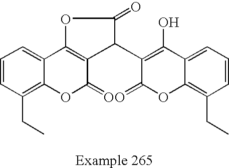 | 77(Method A) | 419.2[M + H]⁺ |
| 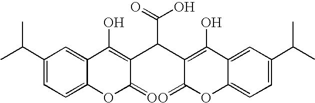 | 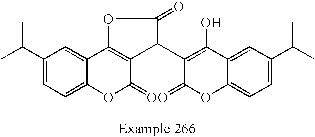 | 52(Method A) | 447.2[M + H]⁺ |
| 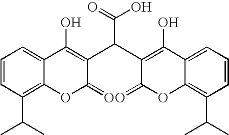 | 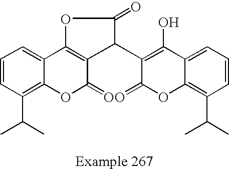 | 50(Method A) | 447.3[M + H]⁺ |
| 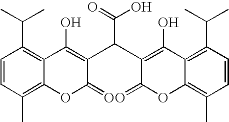 | 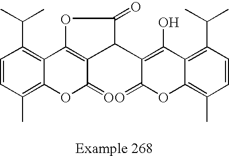 | 39(Method A) | 475.3[M + H]⁺ |
| 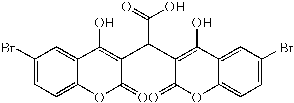 | 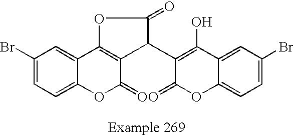 | 54(Method A) | 518.7[M + H]⁺ |
| 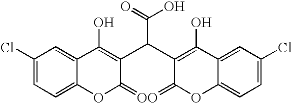 | 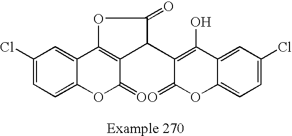 | 89(Method A) | 431.0[M + H]⁺ |
| 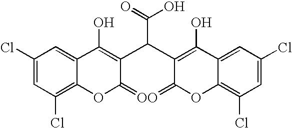 | 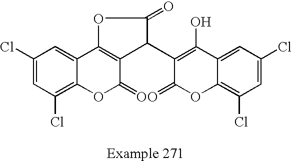 | 56(Method A)79(Method B) | 499.0[M + H]⁺ |
| 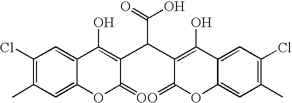 | 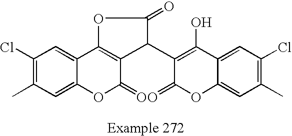 | 81(Method A) | 459.1[M + H]⁺ |

### Examples from 273 to 279

### General Procedures for the Preparation of Compounds of Formula III

wherein D represents —CCH₃ and —— represents a double bond

Method A

A solution of corresponding compound of Formula I, wherein A represents CH—X group, wherein X is a carboxy group and n=0, in Ac₂O was heated to reflux, and stirred under reflux until completion of the reaction (0.5-8 h). After cooling to room temperature (alternatively −18° C.) stirring of the reaction mixture was continued until precipitation occurred. Precipitate was filtered off, washed with cold n-hexane and dried. Alternatively, crude product was purified by chromatography on silica column eluting with CH₂Cl₂/AcOEt (100/0→95/5→9/1→4/1→2/1→1/1). Corresponding acetyl derivative of the compound of Formula III, wherein D represents —CCH3 and —— represents a double bond, was thus formed, which was in the next step subjected to hydrolysis of the acetyl group as described bellow.

To a solution of acetyl derivative of the compound of Formula III, wherein D represents —CCH3 and —— represents a double bond, in AcOH was added water in small portions until solution turned into suspension. The reaction mixture was heated to reflux and stirring under reflux was continued until completion of the hydrolysis (9-48 h). After cooling to room temperature, precipitate was filtered off, washed with ether and dried.

Method B

A solution of corresponding compound of Formula I, wherein A represents CH—X, wherein X is an acetyl group and n=0, in TFAA was heated to reflux, and stirred under reflux until completion of the reaction (2-6 h). After cooling to room temperature, stirring of the reaction mixture was continued, followed by addition of ether and n-hexane until precipitation occurred. Precipitate was filtered off, washed with cold n-hexane and dried.

| | | Yield | MS |
|---|---|---|---|
| Starting compound | Product | (%) | (ESI, m/z) |
| 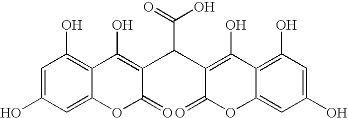 | 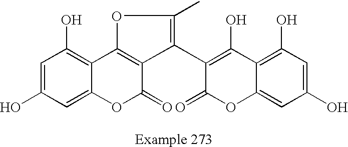 | 12 | 424.9[M + H]⁺ |
| 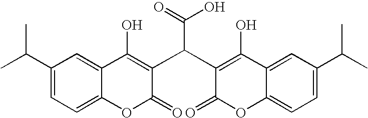 | 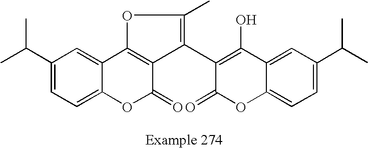 | 5 | 445.1[M + H]⁺ |
| 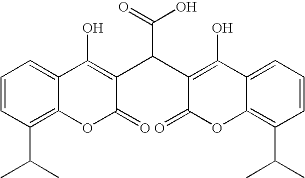 | 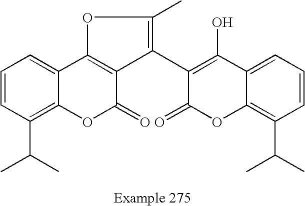 | 47 | 445.2[M + H]⁺ |
| 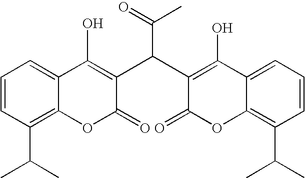 | 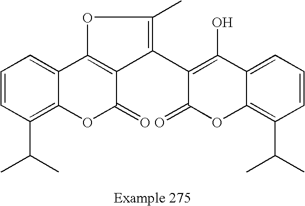 | 80 | 443.0[M − H]⁻ |
| 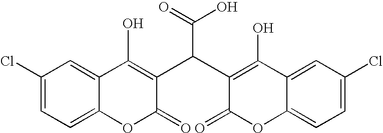 | 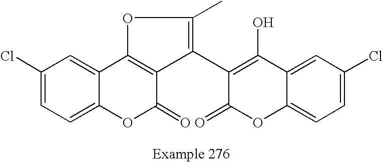 | 55 | 428.9[M + H]⁺ |
| 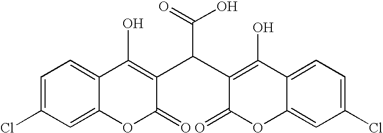 | 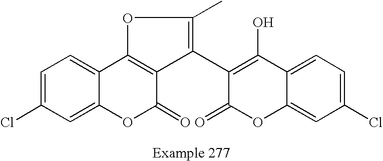 | 53 | 428.9[M + H]⁺ |
| 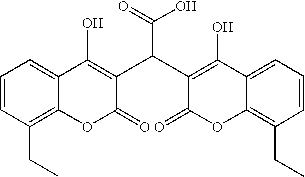 | 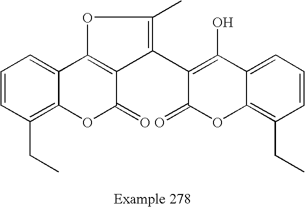 | 43 | 417.1[M + H]⁺ |
| 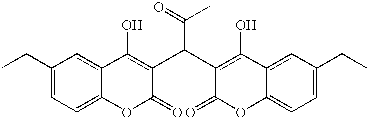 | 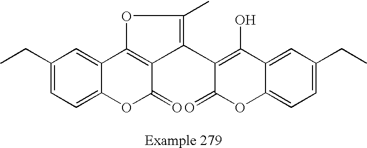 | 68 | 417.0[M + H]⁺ |

### Examples from 280 to 281

### General Procedures for the Preparation of Compounds of Formula III

wherein D represents —CHCH₂OH and —— represents a single bond

Method A

To a suspension of corresponding 4-hydroxycoumarin (1 equiv.) in water was added aq. solution of D,L-glyceraldehyde dimer (0.5 equiv.). The reaction mixture was heated to reflux, and stirring under reflux was continued until completion of the reaction (5-10 h). After isolation of the corresponding compound of Formula I, wherein A represents a carbonyl group and n=1 (Examples 37-41), mother liquor was evaporated to dryness and the product was purified by trituration or recrystallization from MeOH or CHCl₃.

Method B

A solution of corresponding compound of Formula I, wherein A represents CH—X group, wherein X is

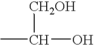

group and n=0, in AcOH was heated to reflux and stirring under reflux was continued 4-10 h, followed by addition of water in small portions. Stirring under reflux was continued until completion of the reaction (24-48 h). After cooling to room temperature, solvent was evaporated under reduced pressure and the crude product was purified by recrystallization from MeOH.

| | | | MS |
|---|---|---|---|
| Starting compound | Product | Yield (%) | (ESI, m/z) |
| 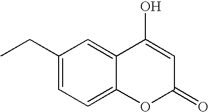 | 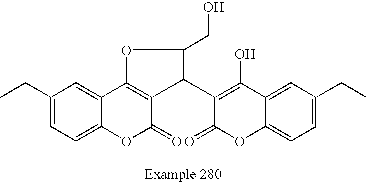 | 4 | 435.0[M + H]⁺ |
| 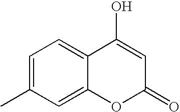 | 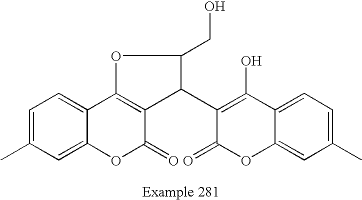 | 2 | 405.1[M − H]⁻ |

### Examples from 282 to 284 and 367 to 370

### General Procedures for the Preparation of Compounds of Formula III

wherein D represents a CH₂ group and —— represents a single bond

Method A

To a suspension of corresponding 4-hydroxycoumarin (1 equiv.) in AcOH (optionally in water) was added 50 wt. % aq. chloroacetaldehyde (4 equiv.). The reaction mixture was heated to reflux, and stirred under reflux until completion of the reaction (1-6 h). After cooling to room temperature, precipitate was filtered off, washed with AcOH, followed by ether and dried. Alternatively, crude product was purified by chromatography on silica column eluting with n-hexane/AcOEt (1:1) and/or recrystallization from MeOH.

Method B

A solution of corresponding compound of Formula I, wherein A represents CH—X group, wherein X is —CH₂OH and n=0, in TFAA was heated to reflux and stirring was continued until completion of the reaction (0.5-5 h). After cooling to room temperature, stirring of the reaction mixture was continued, followed by addition of ether and n-hexane until precipitation occurred. Precipitate was filtered off, washed with cold n-hexane and dried.

| | | | MS |
|---|---|---|---|
| Starting compound | Product | Yield (%) | (ESI, m/z) |
| 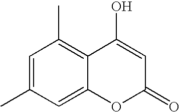 | 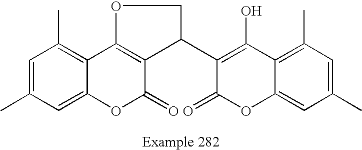 | 45 | 349 [M + H]⁺ |
| 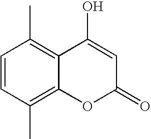 | 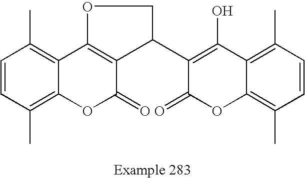 | 20 | 405 [M + H]⁺ |
| 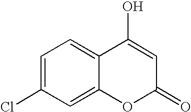 | 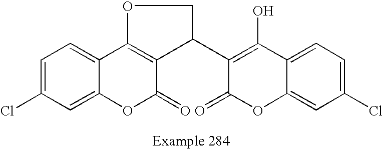 | 21 | 417.1[M + H]⁺ |
| 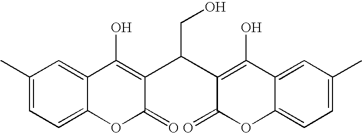 | 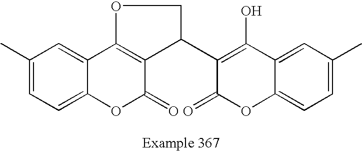 | 32 | 375.0[M − H]⁻ |
| 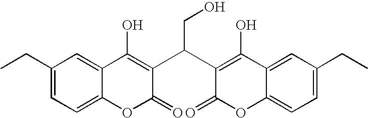 | 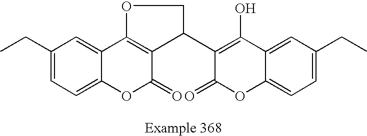 | 23 | 403.0[M − H]⁻ |
| 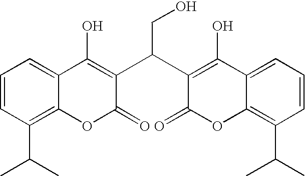 | 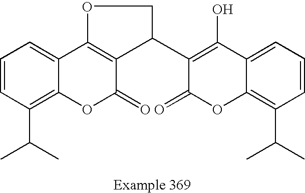 | 22 | 431.0[M − H]⁻ |
| 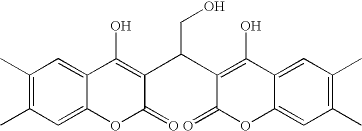 | 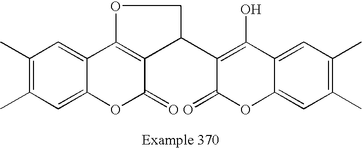 | 43 | 405.0[M + H]⁺ |

### Examples from 371 to 374

### General Procedure for the Preparation of Compounds of Formula III

wherein D represents a CHCH₃ group and —— represents a single bond

A solution of corresponding compound of Formula I, wherein A represents CH—X group, wherein X is

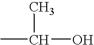

and n=0, in TFAA was heated to reflux and stirring was continued until completion of the reaction (15 minutes to 2 h). After cooling of the reaction mixture solvent was evaporated and the residue was dissolved in MeOH with alternate addition of CHCl₃ or water (up to 1 mL). Stirring of the solution by alternate use of magnetic stirrer and ultrasound bath resulted in precipitation of the product, this was filtered off and dried.

| | | Yield | MS |
|---|---|---|---|
| Starting compound | Product | (%) | (ESI, m/z) |
| 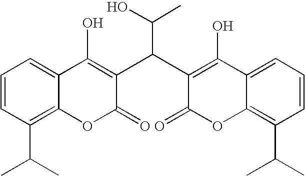 | 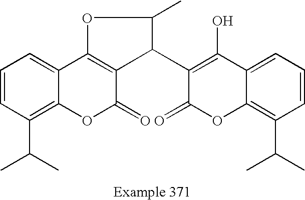 | 43 | 447.0[M + H]⁺ |
| 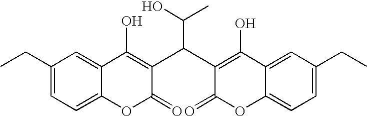 | 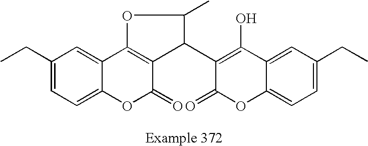 | 51 | 419.0[M + H]⁺ |
| 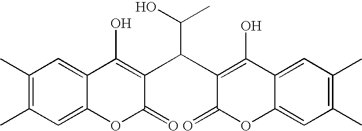 | 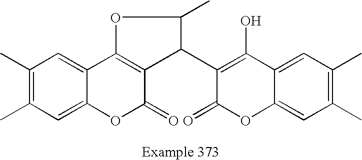 | 46 | 419.0[M + H]⁺ |
| 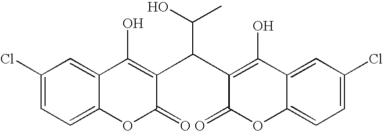 | 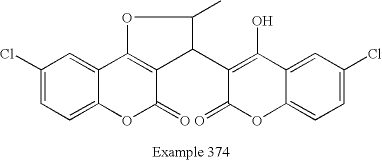 | 69 | 428.9[M − H]⁻ |

### Example 288

### General Procedure for the Preparation of Compounds of Formula III

wherein D represents a CH group and —— represents a double bond

To a suspension of corresponding compound of Formula III, wherein D represents a CH₂ group and —— represents a single bond (0.5 mmol) in tetrachloromethane (10 mL) was added N-bromosuccinimide (0.55 mmol), followed by addition of catalytic amount of benzoylperoxide, and reaction mixture was refluxed for 3 h. Solvent was evaporated under reduced pressure and the remaining crude product was refluxed for 5 min in water, filtered hot and washed with hot water. The crude product was purified by recrystallization from DMF. White product thus obtained was stirred with ether at room temperature for 10 minutes and then filtered off.

| | | | MS |
|---|---|---|---|
| Starting compound | Product | Yield (%) | (ESI, m/z) |
| 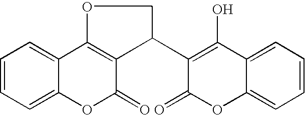 | 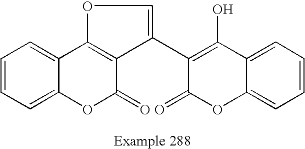 | 40 | 347.1[M + H]⁺ |

Pharmacological Properties

The compounds of Formulae I, II and III possess useful pharmacological properties proved by a number of in vitro and in vivo investigations.

Assays that can be used to determine the anti-inflammatory effects of the compounds and hense, their use for the treatment of diseases characterized by pathologic inflammation are represented by Examples 285 to 287, 289 and 375 to 377. The cytokines assayed in these examples, when expressed at elevated amounts, are markers for inflammation and, in the case of other immune events assayed such as cell proliferation, granulocyte degranulation and lung netrophilia, the behaviors of these immune cells are also markers for their activation and, therefore, inflammation. Consequently, reduction of pro-inflammatory cytokine expression or secretion and reduction in cell proliferation, mast cell degranulation or neutrophil accumulation is a measure of a compound's anti-inflammatory activity. Lung neutrophilia specifically serves as a model for COPD and lung eosinophilia as a model for asthma. Phosphodiesterases are involved in various inflammatory states such as asthma (PDE4), COPD (PDE4) and pulmonary hypertension (PDE3). Prostaglandins and leukotrienes are also potent inflammation mediators, the former being produced in the cyclooxygenase (COX) pathway and the latter in the lipooxygenase pathway. Thromboxanes are also potent inflammation mediators produced in the COX pathway.

A compound analyzed using the biological assays as defined herein is considered to be fully “active” if inhibition is significant (i.e. 50% or higher) in at least one inhibitory function (e.g., inhibition of TNF-α or IL-6) after stimulation with at least one stimulant (e.g., OVA, PMA or LPS), as described for each particular in vitro assay, or if activity in at least one of in vivo testings (e.g. in suppression of ear oedema) is statistically significantly different in comparison to a positive control group, as calculated by the statistical methods known in the art (e.g. ANOVA).

### Example 285

### Inhibition of RBL-2H3 Mast Cell Degranulation

Mast cell degranulation is indicated as invoked in immediate or delayed type hypersensitivity reaction, allergy, anaphylaxis, inflammation, asthma and urticaria (hives).

RBL-2H3 cell line of rat basophilic leukaemia (ATCC) was used for the investigation of inhibition of degranulation induced by the activation of Fcε receptor type I or calcium ionophors. RBL-2H3 cell line was cultivated in DMEM medium (Invitrogen Cat. No. 31966-021) with 10% of phoetal calf serum (Invitrogen Corporation) at 37° C., 5% CO₂, 90% relative humidity. Cells were seeded in the same medium into 24-well plates, 50000 per well, and left to reach 80-90% of confluence.

Dilutions of compounds were prepared in DMEM medium without phenol red (Invitrogen Corporation) in concentrations from 200 μM to 1 μM. The medium was removed from the cells and the diluted of compounds were added to the wells with the exception of the positive and the negative control where pure DMEM medium was added. Subsequently,

1. for the IgE-induced degranulation by Fcs receptor type I, a solution of SPE-1 (dinitrophenyl specific IgE) antibodies (Sigma) and dinitrophenylalbumin (Sigma), both in a final concentration of 0.5 μg/mL,

2. for Ca²⁺-induced degranulation by means of a calcium ionophore, the solution A23187 (Calbiochem) in a final concentration of 250 ng/mL, and

were added to the wells.

In the case of the negative control wells, pure DMEM medium was added. The cells were incubated for one hour at 37° C., 5% CO₂, and 90% relative humidity. Each dilution as well as the positive and the negative controls were performed in triplicate. The supernatant (50 μL) was transferred in duplicate to a 96-well plate. Thereto 100 μL of 50 mM sodium citrate buffer with 1 mg/mL para-nitrophenyl-N-acetyl-β-D-glucosaminide (Calbiochem) were added and it was incubated for 1 hour at 37° C. The reaction was stopped with 100 μL of a saturated sodium carbonate solution. The absorbance was measured at 405 μm. The percentage of inhibition was expressed by the formula:

% inhibition=(1−(OD₄₀₅ sample−OD₄₀₅negative control)/(OD₄₀₅positive control−OD₄₀₅negative control))×100.

Ketotifen, used as a standard, significantly inhibits degranulation in concentrations from 200-50 μM.

Compounds represented by Examples 7, 12, 19, 30, 44-46, 50, 53, 55, 61, 62, 66, 69, 73, 75-83, 85, 86, 88, 89, 93, 94, 116, 143-146, 148, 150, 151, 268, 284, 348, 349-351 and 354 inhibited IgE-induced degranulation by Fcs receptor type I demonstrating significant inhibitory activity in 10 μM concentration.

Compounds represented by Examples 66, 69, 73, 75-77, 80, 82, 88, 89, 94, 96, 97, 101, 103, 105, 106, 108-111, 114, 115, 116, 131, 135, 136, 137, 144, 148, 149, 154, 156, 160, 161, 166, 167, 169-171, 174, 177, 189, 225, 226, 228, 233-235, 237, 238, 241, 243, 246, 309, 314, 315-321, 324, 326, 333,-340, 342-344, 346 and 348 inhibited Ca²⁺-induced degranulation by means of a calcium ionophore demonstrating significant inhibitory activity in 10 μM concentration.

### Example 286a

### Leukotriene B4 (LTB4) Inhibition Assay

Leukotrienes are important mediators in host defense mechanisms and in inflammatory disease states due, for example, to their effects on cell migration, muscle contraction, vascular permeability, and the release of lysozomal enzymes. Leulcotriene production depends on the enzyme activity of 5-lipoxygenase. RBL-2H3 cells have a potent 5-lipoxygenase activity and thus serve as a cell model for production of leulkotrienes.

Compounds were assayed for their ability to inhibit the production of leulcotriene B4 in A23187 stimulated RBL-2H3 cells. RBL-2H3 cell line (ATCC 2256) is grown in DMEM medium (Invitrogen) supplemented with 10% FBS (Invitrogen) in the atmosphere of 5% CO2, 90% humidity, 37 C. Cells are trypsinazed, washed with fresh DMEM medium and adjusted to 1×10⁵ cells per mililiter. 500 μL/well of cell suspension is transferred into 24 well plate (Falcon) and grown overnight in culturing condition described herein. 10 mM solutions of tested compounds are prepared in DMSO (Sigma), and dissolved in working concentrations in DMEM medium without phenol red (Invitrogen). Dilutions of tested compounds are placed on cells, whereas for positive and negative controls only DMEM medium without phenol red are used and left in culturing conditions for 30 minutes. A23187 (Sigma) was added into all wells except negative controls in the final concentration of 250 nM and left for 45 minutes in culturing conditions. 10 μL of cellular supernatant was used to determine leukotriene B4 levels using ELISA (R&D systems). Total concentrations of LTB4 are calculated in samples, and total inhibition was calculated using the formula:

% inhibition=(1−LTB4 sample concentration/LTB4 positive control concentration)×100.

Compounds represented by Examples 7, 17, 18-20, 25, 28-30, 32, 33, 44-47, 49-51, 55, 61, 66, 69, 71, 73, 75-78, 80-83, 86, 88, 93, 94, 96, 97-101, 103, 105, 106, 108-112, 114-116, 123, 125-128, 130, 135-138, 143-145, 148-150, 154-157, 159-162, 164, 166-172, 174, 177-179, 181, 186, 189, 190, 207, 210, 214, 218, 225, 226, 228-235, 237, 238, 241, 243-246, 268, 292-294, 303, 304, 309, 311, 312, 314-321, 324, 326, 332, 333, 334-340, 342-351, 359, 362-364, 369 and 371 demonstrated significant inhibition of LTB4 production at concentrations of 10 μM.

### Example 286b

### 5-Lipoxygenase, 5-LO, Inhibition Assay

5-lipoxygenase is involved in number of immune diseases like asthma and inflammatory bowel disease.

Inhibition of 5-lipoxygenase, 5-LO, was determined by comparison of LTB4 inhibition (Example 286a) and inhibition of Ca²⁺-induced mast cell degranulation (Example 285) in 10 μM concentration. Compounds were considered as 5-LO inhibitors if the difference between inhibitions in these two assays was significant (50% or higher) in favor of LTB4 inhibition. Compounds represented by Examples 17, 19, 20, 25, 29, 30, 32, 44-47, 49, 50, 55, 61, 71, 83, 86, 98, 99, 100, 112, 123, 128, 130, 143, 145, 150, 155, 157, 159, 162, 168, 179, 186, 190, 210, 230, 231, 232, 293, 303, 311, 312, 332, 345, 347, 349-351, 359, 362, 364, 369 and 371 were considered as 5-LO inhibitors according to the above mentioned criterion.

### Example 287

### Evaluation of the Compounds of the Present Invention

All compounds were dissolved in dimethyl sulfoxide (DMSO). Final concentration of the DMSO in all tests was 1% (v/v). Inhibition in all tests is considered preferred if it exceeds 50% at 10 μM concentration (or less).

Human 5-Lipoxygenase, 5-LO, assay

5-lipoxygenase is involved in number of immune diseases like asthma and inflammatory bowel disease.

Human peripheral mononuclear blood cells were isolated using Ficoll density separation. Cells were incubated in Hanks Balanced Salt Solution (HBSS) with compounds for 15 minutes at 37° C. Substrate (arachidonic acid) was added in 20 μM final concentration and cells were incubated for another 15 minutes. LTB4 as a final product of arachidonic acid metabolism was measured using competitive enzyme immunoassay (See, e.g., Safayhi, H. et al. Planta Medica 2000, 66, 110-113) and percentage of inhibition was calculated from the levels of LTB4 in cell supernatant.

Compounds represented by Examples 17, 19, 30, 50, 349 and 351 inhibited human 5-LO demonstrating significantly relevant activity in 10 μM concentration.

Human Leukotriene receptor, Cysteinyl Leukotriene Receptor I (CysLT1), Assay

CysLT1 receptor is involved in immune diseases such as asthma, and has clinical significance as a point of intervention in asthma therapy. CysLT1 receptor is expressed in CHO-K1 cells (Chinese hamster ovary cells K1 clone). This is a competitive radioligand binding assay where substances compete with [³H]-labeled Leulcotirene D4 (LTD4). Radioactive substance is measured with scintillation counting. Percentage of inhibition is calculated from the total radioactivity of the sample.

Compounds represented by Examples 19, 30, 50, 349 and 351 exhibited significantly relevant activity in 10 μM concentration.

Human Phosphodiesterase, PDE, assays

Phosphodiesterases are involved in many cellular processes of signal transduction and have implication in cell growth and division, inflammation, pulmonary hypertension and asthma. Inhibitors of PDE4 have been developed for asthma treatment. The PDE3 inhibitor cilostazol has been approved in certain countries for treatment of arterial occlusive diseases and stroke.

PDE3 assay. Isolated human platelets are pre-incubated at 25° C. in Tris-HCl buffer containing magnesium ions for 15 minutes. 1.01 μM tritium labeled cyclic adenosine monophosphate (cAMP) is added as substrate, followed by incubation at 25° C. for 20 minutes. Percentage of inhibition is calculated by comparing the adenosine levels in supernatant which is formed from cAMP in treated versus control cells.

Compounds represented by Examples 17, 19, 30, 50 and 351 significantly inhibited human PDE3 activity in 10 μM concentration.

PDE4 assay. PDE is implicated in diseases such as chronic obstructive pulmonary disease and neutrophilia. Human monocytic leukemia cell line U937 is pre-incubated at 25° C. in Tris-HCl buffer containing magnesium ions for 15 minutes. 1.01 μM tritium labeled cyclic adenosine monophosphate (cAMP) is added as substrate, followed by incubation at 25° C. for 20 minutes. Percentage of inhibition is calculated by comparing the adenosine levels in supernatant which is formed from cAMP in treated versus control cells. Compounds represented by Examples 17, 19, 30, 50 and 351 significantly inhibited human PDE4 activity in 10 μM concentration.

Human Prostanoid Receptor Assay

Human prostanoid receptor is expressed in Chinese hamster ovary cell line, clone K1 (CHO-K1). Substances and radioactive competitor (tritium labelled Prostaglandin D2 in 1.7 nM concentration) are incubated with cells in HEPES buffer for 2 hours at 25° C. in the presence of manganese ions. Level of the bound PGD2 is measured by scintillation counting, and competition is calculated from comparing the total amount of the bound PGD2 in treated versus control cells. Prostanoids are involved in many physiological and pathological processes, from inflammation to pain.

Compounds represented by Examples 19, 30, 50 and 351 exhibited significantly relevant activity in 10 μM concentration.

Human Thromboxane A2, TxA2, Assay

TxA2 receptor is expressed in HEK-293 cell line. Compounds and radiolabelled competitor SQ-29548 are incubated for 30 minutes at 25° C. Level of bound SQ-29548 is measured by scintillation counting, and competition is calculated from the total amount of the bound SQ-29548. TxA2 receptor is an unstable lipid mediator involved in blood clotting, and immune processes.

Compounds represented by Examples 17, 19, 30, 50 and 349 exhibited significantly relevant activity in 10 μM concentration.

Human Protein Serine/Th Reonine Kinase, ERK1, Assay

Human ERK1 is expressed in Escherichia coli and purified. Compounds are pre-incubated in MOPS/EGTA buffer with pervanadate, DTT and γ-[³²P]-ATP with recombinant enzyme for 15 minutes at 37° C. Substrate (purified myelin basic protein, MBP) is added, followed by incubation at 37° C. for 30 minutes. Enzyme activity is measured by quantitation of [³²P]-MBP and inhibition is calculated from specific radioactivity. ERK1 is a serine/threonine kinase activated in MAPK (mitogen activated kinase) pathway. ERK1 is implicated in a high proportion of human cancers, including Hodgkin disease. ERK1 is involved in processes of cell growth, division and inflammation.

Compound represented by Example 19 significantly inhibited human ERK1 activity at a concentration of 10 μM.

Human Protein Tyrosine Kinase, Lck, inhibition assay

Human recombinant Lck is expressed in insect Sf21 expression system and purified. Substances are pre-incubated for 15 minutes at 25° C. in HEPES buffer containing ATP, pervanadate and magnesium. Substrate (poly Glu-Tyr) is added and incubated for 60 minutes at 25° C. Activity of the enzyme is measured using phosphotyrosine (p-Tyr) specific enzyme immunoassay and inhibition is calculated from the determination of the concentration of p-Tyr. Protein tyrosine kinase, Lck, is a protein tyrosine kinase which is abundantly expressed in T-lymphocytes and is essential for T-cell receptor signal transduction and thus activation of T-lymphocytes. Lck activation stimulates recruitment and activation of ZAP-70, which is essential for T-cell activation. Inhibitors of Lck act as immunoregulatory agents in various models. Lck is implicated in T-cell leukemias and inflammation.

Compound represented by Example 19 significantly inhibited human Lck activity at a concentration of 10 μM.

Hunan Tachykinin NK2 Receptor Assay

Tachykinin NK2 receptor is expressed in Chinese hamster ovary cell line, clone K1 (CHO-K1). Substances and radioactive competitor (tritium labelled SR-48968) are incubated with cells in HEPES/NaOH buffer for 90 minutes at 25° C. in the presence of manganese ions. Levels of the bound SR-48968 is measured by scintillation counting, and competition is calculated from the total amount of the bound SR-48968 in treated and control samples. Tachykinin receptors are involved in physiological and pathological processes of inflammation and pain. Tachykinin NK2 has been identified as a target for intervention in asthma, GI disease, irritable bowel syndrome and pancreatitis. Compounds represented by Examples 19 and 50 exhibited significant inhibition of NK2 activity at 10 μM concentration.

### Example 375

### Inhibition of Cytokine Production

A) Inhibition of Cytokine Production by Stimulated Human White Blood Cells (hWBCs) in Vitro

Stimulated hWBCs were treated with two different concentrations of the compounds (25 μM and 10M). Three different stimuli, inducing inflammatory response through different signalling pathways, were used. Anti-inflammatory activity of the compounds was evaluated based on their ability to inhibit production of pro-inflammatory cytokines (TNF-α, IL-1β, IL-6 and IL-8).

White blood cells were obtained from venous blood of healthy volunteers by sedimentation on 2% dextran T-500 (Amersham Biosciences) and subsequent centrifugations of leukocyte rich plasma. Cells were seeded in a 48-well plate at a concentration of 3-5×10⁶ cells per well and preincubated with the tested compounds for 2 h at 37° C. Afterwards, stimuli (Sigma) were added to the final concentration of 2 μg/mL lipopolysaccharide (LPS), 1 μg/mL phorbol-12-myristate acetate (PMA) or 120 μg/mL zymosan (ZYM). Samples were incubated overnight at 37° C. At the end of incubation supernatants were centrifuged for 10 min at 1500 g and stored at −20° C. until cytokine concentration determination. Cytokines were determined by sandwich ELISA, using capture and detection antibodies (R&D) according to manufacturer's recommendations.

Percentage of inhibition is calculated using formula:

% Inhibition=(1−cytokine concentration of sample/cytokine concentration of positive control)×100.

Compounds are considered active if the percent of inhibition is 50% or greater in concentration of 25 μM or lower.

Compounds which significantly inhibited TNF-α, IL-1β, IL-6 and/or IL-8 production stimulated by PMA, LPS and zymosan are listed in the Table below in concentrations as stated.

| Compound | | | | | | | | | | | | |
|---|---|---|---|---|---|---|---|---|---|---|---|---|
| by | TNF-α stimulated by | | | IL-1β stimulated by | | | IL-6 stimulated by | | | IL-8 stimulated by | | |
| Example | PMA | LPS | ZYM | PMA | LPS | ZYM | PMA | LPS | ZYM | PMA | LPS | ZYM |
| 6 | | | | | | | | | 25 μM | | | |
| 19 | 10 μM | 25 μM | 25 μM | 10 μM | 25 μM | 10 μM | 10 μM | 10 μM | 10 μM | 10 μM | 10 μM | 10 μM |
| 25 | 10 μM | 25 μM | | 10 μM | | | 10 μM | | | 10 μM | | |
| 26 | 25 μM | 25 μM | | | | | 25 μM | | | 25 μM | 25 μM | 25 μM |
| 27 | | | 25 μM | | 10 μM | | | | | | | |
| 28 | | | 25 μM | | 25 μM | | | | | | | |
| 29 | | | 25 μM | | | | | | | | | |
| 30 | 25 μM | 25 μM | 25 μM | 10 μM | 10 μM | 10 μM | | | | | | |
| 50 | 10 μM | 25 μM | 10 μM | 10 μM | 10 μM | 10 μM | 10 μM | 25 μM | 10 μM | 10 μM | 25 μM | 10 μM |
| 51 | 25 μM | 25 μM | 10 μM | 10 μM | | | | | | | | |
| 54 | | | 25 μM | | 25 μM | 25 μM | 10 μM | | 25 μM | 10 μM | | |
| 66 | 25 μM | 25 μM | 25 μM | 25 μM | 25 μM | 25 μM | 10 μM | 25 μM | 25 μM | 25 μM | 10 μM | 25 μM |
| 103 | 10 μM | 25 μM | 25 μM | 10 μM | 25 μM | 25 μM | 10 μM | 25 μM | 25 μM | 10 μM | 25 μM | 25 μM |
| 160 | 25 μM | 25 μM | | 25 μM | 25 μM | | 25 μM | 25 μM | | 25 μM | 25 μM | 25 μM |
| 244 | 25 μM | 25 μM | 25 μM | 25 μM | 25 μM | | 25 μM | 25 μM | 25 μM | 25 μM | 25 μM | 25 μM |
| 245 | 25 μM | 25 μM | 25 μM | | 25 μM | 25 μM | 25 μM | 25 μM | 25 μM | 25 μM | 25 μM | 25 μM |
| 292 | 10 μM | 25 μM | 25 μM | 25 μM | 10 μM | 10 μM | 25 μM | 25 μM | 25 μM | 25 μM | 25 μM | 25 μM |
| 293 | 25 μM | 25 μM | 25 μM | 10 μM | 25 μM | 25 μM | 10 μM | 25 μM | 25 μM | 25 μM | 25 μM | 25 μM |
| 294 | 10 μM | 25 μM | 25 μM | 10 μM | 25 μM | 25 μM | 10 μM | 25 μM | 25 μM | 25 μM | 10 μM | 25 μM |
| 295 | 25 μM | | 25 μM | 10 μM | 10 μM | 10 μM | | | 10 μM | 10 μM | | |
| 349 | 25 μM | | | 10 μM | | | 10 μM | | | | | |
| 351 | 25 μM | | | 25 μM | | | 25 μM | | | 25 μM | | |
| 363 | | | 25 μM | | | 10 μM | | | 25 μM | | | |
| 364 | 25 μM | 25 μM | 25 μM | 25 μM | 25 μM | 25 μM | 25 μM | 25 μM | | 25 μM | 25 μM | 25 μM |

B) Inhibition of Cytokine Production by Lipopolysaccharide (LPS) Stimulated Human Peripheral Blood Mononuclear Cells (hPBMCs) In Vitro

Blood was taken from healthy volunteer donor, diluted with the same volume of saline and was separated by gradient density centrifugation on FicollPaque™ Plus on 400 g for 30 minutes. PBMCs were collected, washed in RPMI, counted and number per mL was adjusted.

Collected PBMCs were cultured into 96 well tissue culture plate, flat bottom as 35.000 cells/well/200 uL in RPMI supplemented with 10% fetal bovine serum, prior inactivated on 56° C. for 30 minutes. Cells were stimulated on IL-1 production by adding LPS (serotype 0111:B4, Sigma, cat# L-2630), at final concentration 1 ng/mL. Unstimulated cells were cultured in medium alone. Stock solution was prepared out of testing compounds as 10 mM in DMSO. Final concentrations made in cell culture medium were tested when they had been added together with LPS. The final DMSO volume ratio in all assays did not exceed 0.1%. Negative and positive control samples were prepared in sextaplicates and samples with tested compound concentrations in triplicates. After overnight incubation in humidified atmosphere containing 5% CO₂, supernatants were collected.

Harvested cell culture supernatants were quantified for IL-1β content by enzyme linked immunosorbent assay (ELISA).

Percentage of inhibition is calculated using formula:

% Inhibition=(1−cytokine concentration of sample/cytokine concentration of positive control)×100.

Compounds are considered active if the percent of inhibition is 50% or greater in concentration of 25 μM or lower.

Compound 244 significantly inhibited IL-1β production in concentration of 25 μM.

C) Inhibition of Cytoline Production by ConcanavalinA (ConA) Stimulated Mouse Splenocytes In Vitro

Spleen cell suspension was obtained from BALB/c mice and lymphocytes separated by gradient density centrifugation on Histopaque 1.083 on 400 g for 30 minutes. They were washed once in medium, counted and their number adjusted as 3×10⁵/200 μL/well in 96 flat bottomed culture plate in RPMI supplemented with 10% fetal bovine serum. Cells were stimulated on cytokine production by adding concanavalinA (ConA) at 5 μg/mL final concentration. Unstimulated cells were cultured in medium alone. Stock solution was prepared out of testing compounds as 10 mM in DMSO. Final concentrations made in cell culture medium were tested when they had been added together with ConA. After 72 hours incubation period cell culture supernatants were collected. In each sample IL-4, IL-5, IL-10 and IFNγ were detected and quantified using enzyme linked immunosorbent assay (ELISA) specific for pointed cytokines (R&D Systems).

To calculate results, standard curve was made out of measured OD values for recombinant protein in known concentrations. Cytokine content in unknown samples was calculated out of OD values extrapolated from the standard curve.

Percentage of inhibition is calculated using formula:

% Inhibition=(1−cytokine concentration of sample/cytokine concentration of positive control)×100.

Compounds are considered active if the percent of inhibition is 50% or greater in concentration of 25 μM or lower.

Compound 244 significantly inhibited IL-4, IL-5, IL-10, as well as IFNγ production in concentration of 25 μM.

Compound 30 significantly inhibited IL-5 and IL-10 production in concentration of 25 μM.

Compounds represented by Examples 19, 295 and 364 significantly inhibited IL-5 production in concentration of 25 μM.

### Example 376

### Phorbol 12-myristate 13-acetate, PMA, Induced Ear Oedema in CD1 Mice

Male CD1 mice (Iffa Credo, France) weighing ˜35-40 g were randomly grouped (n=8 in vehicle treated test group, dexamethasone treated control group as well as in groups treated with compounds to be assayed). Test compounds, dexamethasone as well as vehicle (Trans-phase Delivery System, containing benzyl alcohol 10%, acetone 40% and isopropanol 50%) (all from Kemika, Croatia), were administered topically to the internal surface of the left ear thirty minutes prior to administration of phorbol 12-myristate 13-acetate (PMA) (Alexis biochemicals, USA). Test compounds were administered at a single dose of 250 or 100 μg/15 μL/ear and dexamethasone at a single dose of 50 μg/15 μL/ear. Thirty minutes later, 0.01% PMA solution in acetone was applied topically to the same area of each animal in a volume of 12 μL/ear. During the treatment and challenge, animals were anaesthetized by using inhalation anaesthesia. Six hours after the challenge, animals were euthanized by asphyxiation in 100% CO2 atmosphere. For assessing the auricular oedema, 8 mm discs were cut out of left and right auricular pinna and weighed. The degree of oedema was calculated by subtracting the weight of 8 mm disc of the untreated ear from that of the treated contralateral ear.

The compound at appropriate dose is considered active if the supression of ear oedema in compound treated group is statistically significantly different in comparison to positive control group, as calculated by the statistical methods known from the art (e.g. ANOVA).

Compounds represented by Examples 19, 25, 26, 28, 29, 30, 50, 51, 54, 66, 244, 292, 295, 349, 351, 363 and 364 are considered active according to the above mentioned criterion.

### Example 289

### Model of Ovalbumin Induced Pulmonary Eosinophilia in Mice

Male Balb/C mice with a body weight of 20-25 g are randomly divided into groups, and sensitized by an i.p. injection of ovalbumin (OVA, Sigma) on day zero and day fourteen. On the twentieth day, the mice are subjected to a challenge test by i.n. (intranasal) application of OVA (positive control or test groups) or PBS (negative control). The compounds are administered daily i.n., i.p. or per os in different doses starting 2 days before the challenge and up to the completion of the test. Compounds are administered as suspension either in PBS, methyl cellulose or carboxymethyl cellulose with addition od DMSO (up to 5% of the total volume). 48 hours after i.n. application of OVA, the animals are then anaesthetized to obtain bronchoalveolar lavage fluid (BALF), which is used to determine total protein concentration as well as concentrations of cytokines such as IL-1β and TNF-α, absolute number of cells, and percentage of eosinophils in BALF. Bronchoalveolar lavage (BAL) is performed by infusing 1 mL of PBS divided into 3 separate volumes (0.4, 0.3 and 0.3 ml) through trachea into the lungs. The fluid is immediately withdrawn and the cell suspension stored into previously prepared 1.5 mL Eppendorf tube. Bronchoalveolar lavage fluid (BALF) is then centrifuged at 0.1 g for 5 min (4° C.). Cells are resuspended in 700 μL of PBS. To prepare cytospins, 320 μL of suspension is centrifuged at 250 rpm for 10 min (Cytospin-3, Shandon Instruments). Cells are stained with Dif-Quik (Dade Behring) to determine percentage of eosinophils by counting of at least 100 cells. The remaining resuspended cells are used for total cell count in BALF analysis (Sysmex SF 3000). Finally, lungs are sampled and stored into 10% formalin for pathohistological evaluation of eosinophil and mononuclear infiltrate. Accumulation of eosinophils and mononuclear cells in peribronchial (PB) and perivascular (PV) lung tissue areas and in alveolar spaces is monitored.

Results can be expressed as (I) decrease of absolute cell number per mL in BALF, (II) decrease of number of eosinophils per mL in BALF, (III) reduction of relative number (percentage) of eosinophils in BALF, (IV) reduction of cytokine concentrations in BALF, as well as (V) suppression of accumulation of eosinophils and mononuclear cells in peribronchial (PB) and perivascular (PV) lung tissue areas and in alveolar spaces by pathohistological scoring of treated animals compared to positive control (OVA stimulated, but untreated animals).

The results have to be statistically significantly different when compared to positive control group, as calculated by the statistical methods known in the art (e.g. ANOVA).

Fluticasone and beclomethasone are used as standard anti-inflammatory substances, and compared for ability to inhibit eosinophilia to the negative and positive controls.

Compounds represented by Examples 19, 30, 50 and 363 exhibited statistically significant reduction of percentage of eosinophils and/or their total number in BALF and/or by histological scoring.

### Example 377

### Model of LPS Induced Pulmonary Neutrophilia in Nice

Male Balb/cJ mice (Iffa Credo, France) weighing ˜25 g are randomly grouped into a negative control group, positive control group and groups treated with compounds to be assayed). Test compounds, as well as vehicle (DMSO+0.5% methyl-cellulose) (all from Sigma), are administered i.n., ip. or per os two hours prior to administration of lipopolysaccharide (LPS) (E. coli, serotype 0111:B4, Sigma) or two hours prior and two hours after administration of LPS. Test compounds are administered at a single dose (two hours prior the challenge) or divided into two doses (two hours prior and two hours after the challenge). LPS solution in phosphate buffered saline (PBS) (Sigma) is administered intranasally in volume of 60 μL at concentration of 33,33 μg/mL, to all experimental groups except the negative control group, which received the same volume (60 μL) of vehicle PBS. During the challenge, animals were anaesthetized by using intraperitoneal anaesthesia. Animals are euthanized by i.p. anesthesia overdose approximately 24 hours after application of LPS to obtain bronchoalveolar lavage fluid (BALF), which is used to determine total protein concentration as well as concentrations of cytokines, such as IL-1β and TNF-α, absolute number of cells, and percentage of neutrophils in BALF. Bronchoalveolar lavage (BAL) is performed by infusing 1 mL of PBS divided into 3 separate volumes (0.4, 0.3 and 0.3 ml) through trachea into the lungs. The fluid is immediately withdrawn and the cell suspension stored into previously prepared 1.5 mL Eppendorf tube. Bronchoalveolar lavage fluid (BALF) is then centrifuged at 0.1 g for 5 min (4° C.). Cells are resuspended in 700 μL of PBS. To prepare cytospins, 320 μL of suspension is centrifuged at 250 rpm for 10 min (Cytospin-3, Shandon Instruments). Cells are stained with Dif-Quik (Dade Behring) to determine percentage of neutrophils by counting of at least 200 cells. The remaining resuspended cells are used for total cell count in BALF analysis (Sysmex SF 3000). Finally, lungs are sampled and stored into 10% formalin for pathohistological evaluation of granulocyte and mononuclear infiltrate. Accumulation of granulocytes and mononuclear cells in peribronclhial (PB) and perivascular (PV) lung tissue areas and alveolar spaces is monitored.

Results can be expressed as (I) decrease of absolute cell number per mL in BALF, (II) decrease of number of neutrophils per mL in BALF, (III) reduction of relative number (percentage) of neutrophils in BALF, (IV) reduction of cytokine concentrations in BALF, as well as (V) suppression of accumulation of granulocytes and mononuclear cells in peribronchial (PB) and perivascular (PV) lung tissue areas and in alveolar spaces by pathohistological scoring of treated animals compared to positive control (LPS stimulated, but untreated animals).

The results have to be statistically significantly different when compared to positive control group, as calculated by the statistical methods known in the art (e.g. ANOVA).

Compounds represented by Examples 19, 30, 50 and 363 exhibited statistically significant reduction of percentage of neutrophils and/or their total number in BALF and/or by histological scoring.

1. A compound of Formula I 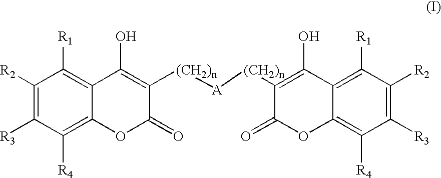
or a pharmaceutically acceptable salt, solvate, tautomer or stereoisomer thereof
wherein:
R₁, R₂, R₃ and R₄ is each independently hydrogen, fluoro, chloro, bromo, C₁-C₄-alkyl, C₂-C₄-alkenyl, C₂-C₄-alkynyl, halo-C₁-C₄-alkyl, hydroxy, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro;
A is carbonyl, CH—X or C═N—R₅;
each occurrence of n is, independently, an integer which is 0 or 1;
R₅ is hydroxy, alkoxy, amino, alkylamino, aryl or arylamino;
X is hydroxy, carboxy, acetyl, alkylcarbonyl, formyl, C₁-C₆ alkyl substituted with one to six hydroxy groups, aryl, heteroaryl, alkyloxycarbonyl, N-alkylcarbamoyl or —C(═N—R₅)R₆; and
R₆ is hydrogen or CH₃;
provided that
i.) when A is carbonyl or C═N—R₅ group then n=1; and
ii.) when n=0 and X is aryl, heteroaryl, formyl, carboxy, acetyl, CH₂OH alkyloxycarbonyl, arylcarbonyl, N-alkylcarbamoyl, 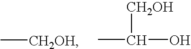
group,
then R₁, R₂, R₃ and R₄ are not all hydrogen; and
iii.) when n=0, R₁=R₂=R₄═H, and R₃═OH or
when n=0, R₂=R₄═H, and R₁=R₃═OH or
when n=0, R₁=R₄═H, and R₂=R₃═OH or
when n=0, R₁=R₂═H, and R₃═OH,
then X is not a formyl or 2-formylphenyl; and
iv.) when n=0, R₁=R₂=R₄═H, and R₃═OH or
when n=0, R₂=R₄═H, and R₁=R₃═OH,
then X is not 4-carboxyphenyl group; and
v.) when n=0, R₁=R₂=R₄═H, and R₃═OH,
then X is not carboxy, phenyl, 4-styrylphenyl, 4-(N,N-dimethylamino)phenyl, 2-pyridinyl, 3-pyridinyl, or 2-naphtalenyl; and
vi.) when n=0, R₁=R₂=R₃═H, and R=Me
then X is not phenyl, 3-bromophenyl, 2-chlorophenyl, 3-chlorophenyl, 4-chlorophenyl, 2,4-dichlorophenyl, 2-methoxyphenyl, 3-methoxyphenyl, 4-methoxyphenyl, 4-nitrorophenyl, 4-hydroxy-3-methoxyphenyl, 4-(N,N-dimethylamino)phenyl, or 4-methylthiophenyl; and
vii.) when n=0, R₁=R₃═R₄═H, and R₂=Me
then X is not phenyl, 2-chlorophenyl, 4-hydroxyphenyl, 2,4-dichlorophenyl, 3-methoxyphenyl, 4-methoxyphenyl 4-hydroxy-3-methoxyphenyl, 3-nitrorophenyl, 2-nitrorophenyl, 2-methoxyphenyl, 3,4,5-trimethoxyphenyl, 4-(N,N-dimethylamino)phenyl, 2-pyridinyl, 3-pyridinyl, 4-pyridinyl, or 2-quinolinyl; and
viii.) when n=0, R₁=R₃═R₄═H, and R₂═F or
when n=0, R₁=R₂=R₄═H, and R₃═F
then X is not carboxy or ethyloxycarbonyl and
ix.) when n=0, R₁=R₂=R₃═H, and R₄═Cl
then X is not phenyl; and
x.) when n=0, R₁=R₃=R₄═H, and R₂═Cl
then X is not phenyl, 4-hydroxyphenyl, 4-methoxyphenyl, 3-nitrophenyl, 4-hydroxy-3-methoxyphenyl, 2-pyridinyl, 3-pyridinyl, 4-pyridinyl, 2-(6-methyl)pyridinyl, or 2-quinolinyl; and
xi.) when n=0, R₁=R₃=R₄═H, and R₂=Br
then X is not phenyl, 2-hydroxyphenyl, 3-hydroxyphenyl, 4-methylphenyl, 4-hydroxy-3-methoxyphenyl, 4-hydroxy-3-ethoxyphenyl, 2-pyridinyl, 4-(N,N-dimethylamino)phenyl, or 5-benzo[1,3]dioxolyl; and
xii.) when n=0, R₁=R₂=R₄═H, and R₃=Me or
when n=0, R₂=R₃═H and R₁=R₄=Me,
then X is not phenyl, 4-hydroxyphenyl, or 4-nitrophenyl; and
xiii.) when n=0, R₂=R₄═H and R₁=R₃=Me,
then X is not phenyl; and
xiv.) when n=0, R₁=R₄═H and R₂═C₁ and R₃=Me,
then X is not phenyl, 4-hydroxyphenyl, 4-methoxyphenyl, 2-(6-methyl)pyridinyl, 3-pyridinyl, 4-pyridinyl, or 2-quinolinyl; and
xv.) when n=0, R₁=R₄═H and R₂=R₃=Me,
then X is not phenyl or 4-methoxyphenyl; and
xvi.) when n=0, R₁=R₃═H and R₂=R₄═Cl,
then X is not phenyl; and
xvii.) when n=0, R₁=R₂═H and R₃═OH and R₄=Me,
then X is not phenyl, 4-methoxyphenyl, 2-hydroxyphenyl, 3-hydroxyphenyl, 4-hydroxy-3-methoxyphenyl, 4-hydroxy-3-ethoxyphenyl, or 5-benzo[1,3]dioxolyl; and
xviii.) when n=1 and X is carboxy or ethyloxycarbonyl
then R₁, R₂, R₃ and R₄ are not all hydrogen; and
xix.) when n=1 and R₁=R₃═R₄═H and R₂=Br,
then X is not phenyl.

2. A compound of formula II 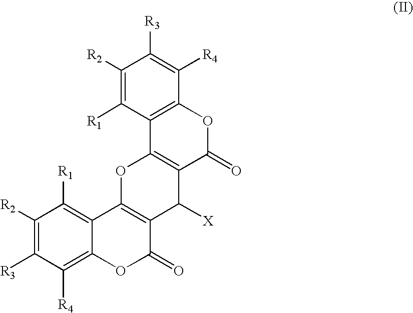
or a pharmaceutically acceptable salt, hydrate, solvate, tautomer or stereoisomer thereof
wherein:
R₁, R₂, R₃ and R₄ is each independently hydrogen, fluoro, chloro, bromo, C₁-C₄-alkyl, C₂-C₄-alkenyl, C₂-C₄-alkynyl, halo-C₁-C₄-alkyl, hydroxy, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro;
X is hydroxy, carboxy, alkylcarbonyl, formyl, C₁-C₆ alkyl substituted with one to six hydroxy groups, aryl, heteroaryl, alkyloxycarbonyl, arylcarbonyl, N-alkylcarbamoyl, or —C(═N—R₅)R₆;
R₅ is hydroxy, alkoxy, amino, alkylamino, aryl or arylamino; and
R₆ is hydrogen or CH₃;
provided that
i.) when X is aryl, heteroaryl, carboxy, acetyl, alkyloxycarbonyl, 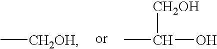
group, then R₁, R₂, R₃ and R₄ are not all hydrogen; and
ii.) when R₁=R₃═R₄═H and R₂=Me,
then X is not 3-(4-hydroxy-6-methyl-2-oxo-2H-1-benzopyranyl).

3. A compound of Formula III 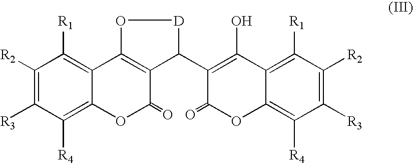
or a pharmaceutically acceptable salt, hydrate, solvate, tautomer or stereoisomer thereof
wherein
R₁, R₂, R₃ and R₄ is each independently hydrogen, fluoro, chloro, bromo, C₁-C₄-alkyl, C₂-C₄-alkenyl, C₂-C₄-alkynyl, halo-C₁-C₄-alkyl, hydroxy, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro;
D is CH, CH₂, CCH₃, CHCH₃, CHCH₂OH, or carbonyl; and
—— is a single or a double bond;
provided that
i.) when D represents CH₂, CHCH₃, CHCH₂OH, or carbonyl group and —— represents a single bond, or when D represents CH or CCH₃ group and —— represents a double bond, then R₁, R₂, R₃ and R₄ are simultaneously not hydrogen;
ii.) when R₁=R₂=R₄═H and R₃═OH, or
when R₂=R₄═H and R₁=R₃═OH, or
when R₁=R₄═H and R₂=R₃═OH, or
when R₁=R₂═H, R₃═OH,
then D is not a CH or CH₂ group.

4. The compound of claim 1 wherein n=1 and A represents carbonyl group.

5. The compound of claim 1 wherein n=0, X is carboxy, acetyl, alkylcarbonyl, arylcarbonyl, C₁-C₆ alkyl substituted with one to six hydroxy groups, alkyloxycarbonyl, N-alkylcarbamoyl, formyl, or —C(═N—R₅)R₆ and at least one of the R₁, R₂, R₃ and R₄ are each independently, chloro, bromo, C₁-C₄-alkyl, C₂-C₄-alkenyl, C₂-C₄-alkynyl, halo-C₁-C₄-alkyl, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro.

6. The compound of claim 5 wherein R₁, R₂, R₃ and R₄ are each independently C₁-C₄-alkyl, chloro or bromo.

7. The compound of claim 1 wherein n=0, X is arylcarbonyl, C₁-C₆ alkyl substituted with one to six hydroxy groups, N-alkylcarbamoyl, or —C(═N—R₅)R₆ and at least one of the R₁, R₂, R₃ and R₄ are each independently, fluoro, chloro, bromo, C₁-C₄-alkyl, hydroxy, C₂-C₄-alkenyl, C₂-C₄-alkynyl, halo-C₁-C₄-alkyl, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano or nitro.

8. The compound of claim 7 wherein R₁, R₂, R₃ and R₄ are each independently C₁-C₄-alkyl, fluoro, chloro, bromo or hydroxy.

9. The compound of claim 1 wherein n=0, X is 2-1H-imidazolyl, 6-(2,3-dihydro)benzo[1,4]dioxinyl, 3-bromo-4-fluorophenyl, 3-bromo-4-methoxyphenyl, 3-ethoxyphenyl, 3-phenoxyphenyl, 4-phenoxyphenyl, 4-benzyloxy-3-methoxyphenyl, 2-(4-bromo)thiophenyl, 4-isopropoxyphenyl, 3-quinolinyl, 4-quinolinyl, 5-(2,3-dihydro)benzofuranyl, 2-furanyl, 4-trifluoromethylphenyl, 2-[5-(3-trifluoromethylphenyl)]furanyl or 2-(5-hydroxymethyl)furanyl and at least one of the R₁, R₂, R₃ and R₄ are each independently, fluoro, chloro, bromo, C₁-C₄-alkyl, hydroxy, C₂-C₄-alkenyl, C₂-C₄-alkynyl, halo-C₁-C₄-alkyl, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro.

10. The compound of claim 9 wherein R₁, R₂, R₃ and R₄ are each independently C₁-C₄-alkyl, fluoro, chloro or bromo.

11. The compound of claim 1 which is:
(a) a compound of formula I, wherein n=0, X is phenyl, R₁=R₂=R₄═H and R₃=Cl;
(b) a compound of formula I, wherein n=0, X is phenyl, R₁=R₂=R₃═H and R₄=isopropyl;
(c) a compound of formula I, wherein n=0, X is 4-methylphenyl, 3-hydroxyphenyl, 4-hydroxyphenyl, 3-chlorophenyl, 4-chlorophenyl, 4-methylthiophenyl, 2-pyridinyl, 2-quinolinyl or 5-benzo[1,3]dioxolyl; R₁=R₄═H and R₂=R₃=Me;
(d) a compound of formula I, wherein n=0, X is 4-methylphenyl, 3-hydroxyphenyl, 4-hydroxyphenyl, 4-chlorophenyl, 4-methylthiophenyl, 2-pyridinyl, 3-pyridinyl, 4-pyridinyl, 2-quinolinyl, 4-methoxyphenyl or 5-benzo[1,3]dioxolyl; R₁=R₃═H and R₂=R₄=Me;
(e) a compound of formula I, wherein n=0, X is 4-hydroxyphenyl, 4-methoxyphenyl, 4-chlorophenyl, 4-methylthiophenyl, 2-quinolinyl, 3-pyridinyl or 4-pyridinyl; R₁=R₃═R₄═H and R₂=Et, F or Br;
(f) a compound of formula I, wherein n=0, X is 4-methylphenyl, 2-pyridinyl, 3-hydroxyphenyl or 5-benzo[1,3]dioxolyl; R₁=R₃═R₄═H and R₂=Et or F;
(g) a compound of formula I, wherein n=0, X is 4-methylphenyl, 3-hydroxyphenyl, 3-chlorophenyl, 4-chlorophenyl, 4-methylthiophenyl or 5-benzo[1,3]dioxolyl; R₁=R₃═R₄═H and R₂═Cl;
(h) a compound of formula I, wherein n=0, X is 4-methylphenyl, 3-hydroxyphenyl, 4-chlorophenyl, 4-methylthiophenyl, 2-pyridinyl, 4-pyridinyl, 2-quinolinyl or 5-benzo[1,3]dioxolyl; R₁=R₂=R₄═H and R₃=Me;
(i) a compound of formula I, wherein n=0, X is 4-methylphenyl, 3-hydroxyphenyl, 3-chlorophenyl, 4-chlorophenyl, 4-methylthiophenyl, 2-pyridinyl or 5-benzo[1,3]dioxolyl; R₁=R₄═H; R₂═C₁ and R₃=Me;
(j) a compound of formula I, wherein n=0, X is 5-benzo[1,3]dioxolyl; R₁=R₃═H and R₂=Me;
(k) a compound of formula I, wherein n=0, X is carboxy; R₁=Me; R₂=R₄═H and R₃═OH; or
(l) a compound of formula I, wherein n=0, X is acetyl; R₁=R₃═OH and R₂=R₄═H.

12. The compound of claim 2 wherein X is carboxy, acetyl, alkylcarbonyl, arylcarbonyl, formyl, C₁-C₆ alkyl substituted with one to six hydroxy groups, aryl, alkyloxycarbonyl, N-alkylcarbamoyl or —C(═N—R₅)R₆ and at least one of the R₁, R₂, R₃ and R₄ are each independently, fluoro, chloro, bromo, C₁-C₄-alkyl, hydroxy, C₂-C₄-alkenyl, C₂-C₄-alkynyl, halo-C₁-C₄-alkyl, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro.

13. The compound of claim 12 wherein R₁, R₂, R₃ and R₄ are each independently C₁-C₄-alkyl, fluoro, chloro or bromo.

14. The compound of claim 2 wherein X is carboxy, acetyl, formyl, C₁-C₆ alkyl substituted with one to six hydroxy groups and at least one of the R₁, R₂, R₃ and R₄ are each independently, C₁-C₄-alkyl, fluoro, chloro or bromo.

15. The compound of claim 3 wherein
D is CHCH₃, CHCH₂OH or carbonyl and —— is a single bond;
or D is CCH₃ and —— is a double bond;
and at least one of the R₁, R₂, R₃ and R₄ are each independently fluoro, chloro, bromo, C₁-C₄-alkyl, hydroxy, C₂-C₄-alkenyl, C₂-C₄-alkynyl, halo-C₁-C₄-alkyl, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro.

16. The compound of claim 15 wherein R₁, R₂, R₃ and R₄ are each independently C₁-C₄-alkyl, fluoro, chloro or bromo or hydroxy.

17. The compound of claim 3 wherein
D is CH or CH₂;
and at least one of the R₁, R₂, R₃ and R₄ are each independently, fluoro, chloro, bromo, C₁-C₄-alkyl, C₂-C₄-alkenyl, C₂-C₄-alkynyl, halo-C₁-C₄-alkyl, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro.

18. The compound of claim 17 wherein R₁, R₂, R₃ and R₄ are each independently C₁-C₄-alkyl, fluoro, chloro or bromo.

19. A pharmaceutical composition comprising one or more compounds as set forth in claim 1 and one or more pharmaceutically acceptable diluents, carriers or excipients.

20. A method for treatment of an inflammatory condition or an immune disorder associated with infiltration of leukocytes into inflamed tissue in a subject in need thereof which comprises administering to said subject a therapeutically effective amount of a compound of Formula VI 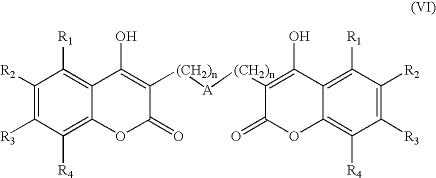
or a pharmaceutically acceptable salt, solvate, tautomer or stereoisomer thereof
wherein:
R₁, R₂, R₃ and R₄ is each independently hydrogen, halogen, C₁-C₄-alkyl, C₂-C₄-alkenyl, C₂-C₄-alkynyl, halo-C₁-C₄-alkyl, hydroxy, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro;
A is carbonyl, CH—X or C═N—R₅;
each occurrence of n is, independently, an integer which is 0 or 1;
R₅ is hydroxy, alkoxy, amino, alkylamino, aryl or arylamino;
X is hydrogen, hydroxy, carboxy, acetyl, alkylcarbonyl, formyl, C₁-C₆ alkyl substituted with one to six hydroxy groups, aryl, heteroaryl, alkyloxycarbonyl, N-alkylcarbamoyl, or —C(═N—R₅)R₆; and
R₆ is hydrogen or CH₃.

21. A method for treatment of an inflammatory condition or an immune disorder associated with infiltration of leukocytes into inflamed tissue in a subject in need thereof which comprises administering to said subject a therapeutically effective amount of a compound of Formula VII 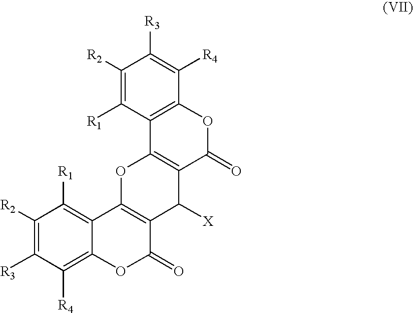
or a pharmaceutically acceptable salt, hydrate, solvate, tautomer or stereoisomer thereof
wherein:
R₁, R₂, R₃ and R₄ is each independently hydrogen, halogen, C₁-C₄-alkyl, C₂-C₄-alkenyl, C₂-C₄-alkynyl, halo-C₁-C₄-alkyl, hydroxy, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro;
X is hydrogen, hydroxy, carboxy, alkylcarbonyl, formyl, C₁-C₆ alkyl substituted with one to six hydroxy groups, aryl, heteroaryl, alkyloxycarbonyl, N-alkylcarbamoyl, or —C(═N—R₅)R₆;
R₅ is hydroxy, alkoxy, amino, alkylamino, aryl or arylamino; and
R₆ is hydrogen or CH₃

22. A method for treatment of an inflammatory condition or an immune disorder associated with infiltration of leukocytes into inflamed tissue in a subject in need thereof which comprises administering to said subject a therapeutically effective amount of a compound of Formula VIII 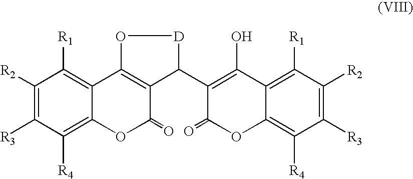
or a pharmaceutically acceptable salt, hydrate, solvate, tautomer or stereoisomer thereof
wherein
R₁, R₂, R₃ and R₄ is each independently hydrogen, halogen, C₁-C₄-alkyl, C₂-C₄-alkenyl, C₂-C₄-alkynyl, halo-C₁-C₄-alkyl, hydroxy, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro;
D is CH, CH₂, CCH₃, CHCH₃, CHCH₂OH, or carbonyl; and
—— is a single or a double bond.

23. The method according to claim 1, wherein the inflammatory condition or immune disorder is selected from asthma; chronic obstructive pulmonary disease; bronchitis; adult respiratory distress syndrome; nasal inflammatory diseases selected from allergic rhinitis, nasal polyps; inflammatatory skin disorders selected from eczemas, psoriasis, allergic dermatitis, neurodermatitis, pruritis, conjunctivitis; rheumatoid arthritis; inflammatory bowel diseases selected from Crohn's disease, colitis and ulcerative colitis; further insulin-dependent diabetes, autoimmune diseases selected from thyroiditis, lupus erythematosus, multiple sclerosis, Raynaud's disease, and other arthritic conditions having an inflammatory component selected from rheumatoid spondylitis, septic arthritis, polyarthritis, retinitis, inflammatory brain disorders selected from meningitis and encephalitis; conditions associated with acute trauma selected from cerebral injury, heart tissue injury and lung injury; inflammation accompanying infections selected from sepsis and nephritis.

24. The method according to claim 23, wherein inflammatory condition or immune disorder is asthma, chronic obstructive pulmonary disease, adult respiratory distress syndrome, bronchitis, allergic rhinitis, nasal polyps, eczemas, psoriasis, allergic dermatitis, neurodermatitis, pruritis, conjunctivitis, rheumatoid arthritis, or an inflammatory bowel disease.

25. A method for inhibiting or reducing inflammation in an affected organ or tissue comprising delivering to said organ or tissue a therapeutically effective amount of the compound of Formula VI 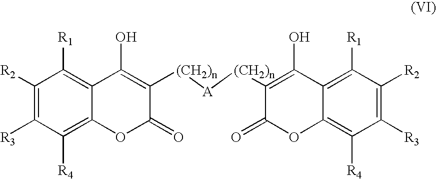
or a pharmaceutically acceptable salt, solvate, tautomer or stereoisomer thereof
wherein:
R₁, R₂, R₃ and R₄ is each independently hydrogen, halogen, C₁-C₄-alkyl, C₂-C₄-alkenyl, C₂-C₄-alkynyl, halo-C₁-C₄-alkyl, hydroxy, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro;
A is carbonyl, CH—X or C═N—R₅;
each occurrence of n is, independently, an integer which is 0 or 1;
R₅ is hydroxy, alkoxy, amino, alkylamino, aryl or arylamino;
X is hydrogen, hydroxy, carboxy, acetyl, alkylcarbonyl, formyl, C₁-C₆ alkyl substituted with one to six hydroxy groups, aryl, heteroaryl, alkyloxycarbonyl, N-alkylcarbamoyl, or —C(═N—R₅)R₆; and
R₆ is hydrogen or CH₃.

26. A method for inhibiting or reducing inflammation in an affected organ or tissue comprising delivering to said organ or tissue a therapeutically effective amount of the compound of Formula VII 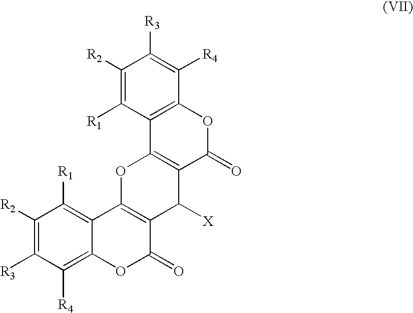
or a pharmaceutically acceptable salt, hydrate, solvate, tautomer or stereoisomer thereof
wherein:
R₁, R₂, R₃ and R₄ is each independently hydrogen, halogen, C₁-C₄-alkyl, C₂-C₄-alkenyl, C₂-C₄-alkynyl, halo-C₁-C₄-alkyl, hydroxy, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro;
X is hydrogen, hydroxy, carboxy, alkylcarbonyl, formyl, C₁-C₆ alkyl substituted with one to six hydroxy groups, aryl, heteroaryl, alkyloxycarbonyl, N-alkylcarbamoyl, or —C(═N—R₅)R₆;
R₅ is hydroxy, alkoxy, amino, alkylamino, aryl or arylamino; and
R₆ is hydrogen or CH₃

27. A method for inhibiting or reducing inflammation in an affected organ or tissue comprising delivering to said organ or tissue a therapeutically effective amount of the compound of Formula VIII 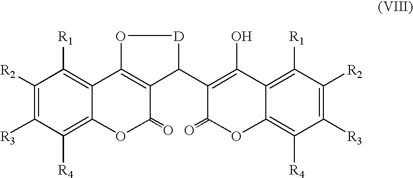
or a pharmaceutically acceptable salt, hydrate, solvate, tautomer or stereoisomer thereof
wherein
R₁, R₂, R₃ and R₄ is each independently hydrogen, halogen, C₁-C₄-alkyl, C₂-C₄-alkenyl, C₂-C₄-alkynyl, halo-C₁-C₄-alkyl, hydroxy, C₁-C₄-alkoxy, trifluoromethoxy, C₁-C₄-alkanoyl, amino, amino-C₁-C₄-alkyl, N—(C₁-C₄-alkyl)amino, N,N-di(C₁-C₄-alkyl)amino, mercapto, C₁-C₄-alkylthio, sulfo, C₁-C₄-alkylsulfo, sulfino, C₁-C₄-alkylsulfino, carboxy, C₁-C₄-alkoxycarbonyl, cyano, or nitro;
D is CH, CH₂, CCH₃, CHCH₃, CHCH₂OH, or carbonyl; and
—— is a single or a double bond.

28. The compound of claim 1 wherein the compound is selected from the group of compounds of Examples 1-284, 288, 290-374.

29. A compound of Formula I 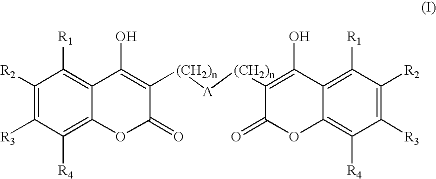
or a pharmaceutically acceptable salt, solvate, tautomer or stereoisomer thereof.
wherein:
R₁, R₂, R₃ and R₄ is each independently hydrogen, fluoro, chloro, bromo, C₁-C₃-alkyl, hydroxy, or nitro;
A is carbonyl or CH—X,
each occurrence of n is, independently, an integer which is 0 or 1; and
X is carboxy, —C(O)O(C₁₋₄alkyl), —CH(OH)CH₂OH, —CH2OH, C(O)OCH₂CH2OH, C(O)NHdecyl, C(O)NH(CH₂)₃OH, C(O)NHC(CH₃)₂CH₂OH, C(O)NHC(CH₂OH)₃, 1-hydroxyethyl, —C(═N)—OH, imidazolyl, phenyl, 2-methoxyphenyl, 4-quinolinyl, 2-quinolinyl, 2-nitrophenyl, 3-chlorophenyl, 2,4-dimethoxyphenyl, 4-(1-butyl)-imidazolyl, naphthyl, 2-hydroxy-4-nitrophenyl, 6-(2,3-dihydrobenzodioxanyl), 2-(4-chlorophenylthio)-phenyl, 2-pyridinyl, 3-bromo-4-fluorophenyl, 3-bromo-4-methoxyphenyl, 3-chloro-4-fluorophenyl, 3-ethoxyphenyl, 3-hydroxyphenyl, 3-phenoxyphenyl, 3-methoxy-4-benzyloxyphenyl, 4-thiomethylphenyl, 2-(4-bromothiophenyl), 4-chlorophenyl, 4-cyanophenyl, 4-isopropoxyphenyl, 4-methylphenyl, 4-phenoxyphenyl, 2-(5-methyl)-furanyl, 3,4,5-trimethoxyphenyl, 4-fluorophenyl, 4-pyridyl, 2-chlorophenyl, 1-imidazolyl, 3,4-methylenedioxyphenyl, 4-methoxyphenyl, 3-quinolinyl, 2-quinolinyl, 4-hydroxyphenyl, 5-(2,3-dihydro)-benzofuranyl, 2-methoxyphenyl, 2-furanyl, 4-trifluoromethylphenyl, 2-[5-(3-trifluoromethylphenyl)]-furanyl, or 1-(5-hydroxymethyl)-furanyl; and
provided that
i.) when A is carbonyl then n=1; and
ii.) when n=0 and X is carboxy, —C(O)O(C₁₋₄alkyl), —CH(OH)CH₂OH, —CH2OH, C(O)OCH₂CH2OH, C(O)NHdecyl, C(O)NH(CH₂)₃OH, C(O)NHC(CH₃)₂CH₂OH, C(O)NHC(CH₂OH)₃, imidazolyl, phenyl, 2-methoxyphenyl, 4-quinolinyl, 2-quinolinyl, 2-nitrophenyl, 3-chlorophenyl, 2,4-dimethoxyphenyl, 4-(1-butyl)-imidazolyl, naphthyl, 2-hydroxy-4-nitrophenyl, 6-(2,3-dihydrobenzodioxanyl), 2-(4-chlorophenylthio)-phenyl, 2-pyridinyl, 3-bromo-4-fluorophenyl, 3-bromo-4-methoxyphenyl, 3-chloro-4-fluorophenyl, 3-ethoxyphenyl, 3-hydroxyphenyl, 3-phenoxyphenyl, 3-methoxy-4-benzyloxyphenyl, 4-thiomethylphenyl, 2-(4-bromothiophenyl), 4-chlorophenyl, 4-cyanophenyl, 4-isopropoxyphenyl, 4-methylphenyl, 4-phenoxyphenyl, 2-(5-methyl)-furanyl, 3,4,5-trimethoxyphenyl, 4-fluorophenyl, 4-pyridyl, 2-chlorophenyl, 1-imidazolyl, 3,4-methylenedioxyphenyl, 4-methoxyphenyl, 3-quinolinyl, 2-quinolinyl, 4-hydroxyphenyl, 5-(2,3-dihydro)-benzofuranyl, 2-methoxyphenyl, 2-furanyl, 4-trifluoromethylphenyl, 2-[5-(3-trifluoromethylphenyl)]-furanyl, or 1-(5-hydroxymethyl)-furanyl; then R₁, R₂, R₃ and R₄ are not all hydrogen; and
iii.) when n=0, R₁=R₂=R₄═H, and R₃═OH,
then X is not carboxy, phenyl, 2-pyridinyl, or 2-naphtalenyl; and
iv.) when n=0, R₁=R₂=R₃═H, and R=Me
then X is not phenyl, 2-chlorophenyl, 3-chlorophenyl, 4-chlorophenyl, 2-methoxyphenyl, 4-methoxyphenyl, or 4-methylthiophenyl; and
v.) when n=0, R₁=R₃=R₄═H, and R₂=Me
then X is not phenyl, 2-chlorophenyl, 4-hydroxyphenyl, 4-methoxyphenyl, 2-methoxyphenyl, 3,4,5-trimethoxyphenyl, 2-pyridinyl, 4-pyridinyl, or 2-quinolinyl; and
vi.) when n=0, R₁=R₃=R₄═H, and R₂═F or
when n=0, R₁=R₂=R₄═H, and R₃═F
then X is not carboxy, or ethyloxycarbonyl; and
vii.) when n=0, R₁=R₂=R₃═H, and R₄═Cl
then X is not phenyl; and
viii.) when n=0, R₁=R₃═R₄═H, and R₂═Cl
then X is not phenyl, 4-hydroxyphenyl, 4-methoxyphenyl, 2-pyridinyl, 4-pyridinyl, or 2-quinolinyl; and
ix.) when n=0, R₁=R₃=R₄═H, and R₂=Br
then X is not phenyl, 3-hydroxyphenyl, 4-methylphenyl, 2-pyridinyl, or 5-benzo[1,3]dioxolyl; and
x.) when n=0, R₁=R₂=R₄═H, and R₃=Me or
when n=0, R₂=R₃═H and R₁=R₄=Me,
then X is not phenyl, or 4-hydroxyphenyl; and
xi.) when n=0, R₂=R═H and R₁=R₃=Me,
then X is not phenyl; and
xii.) when n=0, R₁=R₄═H and R₂═Cl and R₃=Me,
then X is not phenyl, 4-hydroxyphenyl, 4-methoxyphenyl, 4-pyridinyl, or 2-quinolinyl; and
xiii.) when n=0, R₁=R₄═H and R₂=R₃=Me,
then X is not phenyl, or 4-methoxyphenyl; and
xiv.) when n=0, R₁=R₃═H and R₂=R₄═C1,
then X is not phenyl; and
xv.) when n=0, R₁=R₂═H and R₃═OH and R₄=Me,
then X is not phenyl, 4-methoxyphenyl, 3-hydroxyphenyl, or 5-benzo[1,3]dioxolyl; and
xvi.) when n=1 and X is carboxy or ethyloxycarbonyl
then R₁, R₂, R₃ and R₄ are not all hydrogen; and
xvii.) when n=1 and R₁=R₃═R₄═H and R₂=Br,
then X is not phenyl.

30. (canceled)

31. (canceled)

32. A method for inhibition of at least one inflammation marker selected from the group consisting of granulocyte (e.g., mast cell) degranulation, LTB4 production, 5-lipoxygenase production, CysLT1 receptor, PDE3 activity, PDE4 activity, binding to human prostanoid receptor, binding to human thromboxane receptor, protein serine/threonine kinase ERKF1, activity protein tyrosine kinase LCK activity, binding to tachykinin receptor, production of a least one pro-inflammatory cytokine selected from the group consisting of TNF-α, IL-1β, IL-2, IL-5, IL-6, and IL-8, oedema, eosinophilia, interferon-γ and neutrophilia, the method comprising exposing an organ or cell tissue afflicted with inflammation to an amount of a compound according to claim 1 effective to inhibit said inflammation marker.

33. The method of claim 32 wherein said marker is mast cell degranulation and is associated with immediate or delayed hypersensitivity, allergy, anaphylaxis, inflammation, asthma or urticaria.

34. The method of claim 32 wherein said marker is LTB-4 production and is associated with inflammation.

35. The method of claim 32 wherein said marker is 5-lipoxygenase and is associated with asthma or inflammatory bowel disease.

36. The method of claim 32 wherein said marker is CysLT1 receptor and is associated with asthma.

37. The method of claim 32 wherein said marker is PDE3 and is associated with cancer, inflammation, pulmonary hypertension or stroke.

38. The method of claim 32 wherein said marker is PDE4 and is associated with chronic obstructive pulmonary disease, neutrophilia or asthma.

39. The method of claim 32 wherein said marker is binding to prostanoid receptor and is associated with inflammation or asthma.

40. The method of claim 32 wherein said marker is inhibition of ERK1 kinase and is associated with cancer or inflammation.

41. The method of claim 32 wherein said marker is LCK kinase activity and is associated with diseases of T-cell activation T-cell leukemia and T-cell mediated inflammatory responses.

42. The method of claim 32 wherein said marker is binding to tachykinin NK2 receptor and is associated with asthma, gastrointestinal disease, irritable bowel syndrome or pancreatitis.

43. The method of claim 32 wherein said marker is cytokine production and is associated with inflammation.

44. The method of claim 32 wherein said marker is oedema.

45. The method of claim 32 wherein said marker is pulmonary eosinophelia in mice and is associated with asthma.

46. The method of claim 32 wherein said marker is pulmonary neutrophilia in mice and is associated with COPD.

47. The compound of claim 1 wherein the compound is selected from the group consisting of the compounds of Examples 1-54, 59, 95, 96, 98, 99, 101, 102, 103, 105-112, 114, 115, 117, 118, 120, 120, 122-124, 126-136, 138-141, 144, 147-149, 151, 153-172, 174-177, 179-222, 224-284, 291-342 and 356-374.

48. The compound of claim 1, wherein it is further provided that;
(i) when n=0, R₁=R₃=R₄═H, and R₂=Br
then X is not 4-hydroxyphenyl or 3,4-methylenedioxyphenyl; and
(ii) when n=0, R₁=R₂=R₄═H, and R₃═Cl
then X is not phenyl.

48. The compound of claim 29, wherein it is further provided that;
(i) when n=0, R₁=R₃=R₄═H, and R₂=Br
then X is not 4-hydroxyphenyl or 3,4-methylenedioxyphenyl; and
(ii) when n=0, R₁=R₂=R₄═H, and R₃═Cl
then X is not phenyl.
